(19) United States
(12) Patent Application Publication
(10) Pub. No.: US 2026/0265274 A1
(43) Pub. Date: Sep. 10, 2026

(54) Cyclic Boronic Esters and Their Uses

(71) Applicant: Principia Biopharma Inc., Bridgewater, NJ (US)

(72) Inventors: Pasit Phiasivongsa, San Francisco, CA (US); Kolbot By, San Ramon, CA (US); Jiang Zhu, San Francisco, CA (US)

(73) Assignee: Principia Biopharma Inc., Bridgewater, NJ (US)

(21) Appl. No.: 18/876,345

(22) PCT Filed: Jun. 28, 2023
(86) PCT No.: PCT/US2023/026427

§ 371 Date: Dec. 18, 2024

Related U.S. Application Data
(60) Provisional application No. 63/357,093, filed on Jun. 30, 2022.

(51) Int. Cl. C07F 5/02; A61K 31/69; A61P 37/00

(52) U.S. Cl. CPC C07F 5/025; A61K 31/69; A61P 37/00

(57) ABSTRACT

The present disclosure relates to cyclic boronic esters and salts thereof for use as immunoproteasome inhibitors. The disclosure further relates to pharmaceutical compositions, methods of preparing, crystal and polymorph forms, and methods of treatment relating to the same.

### CROSS-REFERENCE TO RELATED APPLICATIONS

This application claims priority to U.S. Provisional Application No. 63/357,093, filed on Jun. 30, 2022, which is hereby incorporated by reference in its entirety.

### FIELD OF INVENTION

The present disclosure relates to cyclic boronic esters and salts thereof for use as immunoproteasome inhibitors. The disclosure further relates to pharmaceutical compositions, methods of preparing, crystal and polymorph forms, and methods of treatment relating to the same.

### BACKGROUND OF THE DISCLOSURE

The immunoproteasome is a large proteolytic machinery derived from the constitutive proteasome and is expressed in both immune cells and nonimmune cells. The immunoproteasome plays a critical role in the immune system because it degrades proteins into short peptides that can be processed to fit into the groove of major histocompatibility complex (MHC) class I molecules. This display of peptides on MHC I allows recognition by CD8 T cells of the adaptive immune system. Such recognition can aid in the destruction of pathogen-infected cells by the CD8 T cells. In addition to the immunoproteasome's role in pathogen recognition and clearance, the immunoproteasome is also known to influence inflammatory disease progression through its ability to regulate T cell polarization, and inflammation and neoplastic transformation. See, e.g., Kimura, H. et al., 2015. “New Insights into the Function of the Immunoproteasome in Immune and Nonimmune Cells,” J Immunology Research, vol. 2015, Article ID 541984, 8 pages; Ogorevc, E., 2018. “A patent review of immunoproteasome inhibitors,” Expert Opin. on Therapeutic Patents, vol. 28, no. 7, 517-540.

Two important immunoproteasome subunits are known as large multifunctional peptidase 2 (LMP2 or iβ1) and large multifunctional peptidase 7 (LMP7 or iβ5). See, e.g., Kimura 2015. Both subunits have been implicated in pathways relating to various autoimmune, inflammatory and hematological disorders. For example, both LMP2 and LMP7 were upregulated in sporadic inclusion body myositis, immune-mediated necrotizing myopathies and dermatomyositis muscular biopsies and colocalized with the MHC class I expressing myofibers. See Bhattarai, S. et al. 2016, “The immunoproteasomes are key to regulating myokines and MHC class I expression in idiopathic inflammatory myopathies,” J Autoimmunity, 75:118-129. Additionally, LMP2 and LMP7 expression was also shown to be upregulated in colitis lesions. See, e.g., id.; Schmidt, N., et al. 2010. “Targeting the proteasome: partial inhibition of the proteasome by bortezomib or deletion of the immunosubunit LMP7 attenuates experimental colitis,” Gut 59:896-906. Genetic variations of immunoproteasome subunits LMP2 and LMP7 have been associated with psoriasis based on analysis of patient samples. See, e.g., Krämer, U. et al. 2007. “Strong associations of psoriasis with antigen processing LMP and transport genes TAP differ by gender and phenotype,” Genes and Immunity 8, 513-51.

Various immunoproteasome inhibitors are known, for example, PKS3053 is a LMP7 selective inhibitor that decreased inflammation, cellular infiltration, and tissue damage in a mouse model of skin injury, suggesting LMP7 inhibition may be a potential therapy for inflammatory skin diseases such as psoriasis, cutaneous lupus erythematosus, and systemic sclerosis. See Ah Kioon, M. D. et al. 2021. “Noncytotoxic Inhibition of the Immunoproteasome Regulates Human Immune Cells In Vitro and Suppresses Cutaneous Inflammation in the Mouse,” J Immunol. 206:1631-1641. ONX-0914 (also known as PR-957), a selective LMP7 inhibitor, has been used as a treatment for autoimmune diseases such as colitis in animal models. See, e.g., Kimura 2015 (above). Studies on ONX-0914 have also suggested the potential to attenuate inflammatory bowel disease (IBD) including Crohn's disease and ulcerative colitis. See Basler M. et al. 2010 “Prevention of Experimental Colitis by a Selective Inhibitor of the Immunoproteasome,” J Immunol. 185 (1): 634-641. ONX-0914 has been reported to attenuate experimental arthritis by blocking inflammatory cytokine expression. See, e.g., Kimura 2015 (above). ONX-0914 was also found to ameliorate graft-versus-host disease (GVHD) in certain murine models. See Zilberberg, J. et al. 2015 “Inhibition of the Immunoproteasome Subunit LMP7 with ONX0914 Ameliorates Graft-versus-Host Disease in an MHC-Matched Minor Histocompatibility Antigen-Disparate Murine Model,” Biol. Blood Marrow Transplant., 21 (9): 1555-64. KZR-616 is a tripeptide epoxyketone that selectively and irreversibly inhibits the LMP7 and LMP2. See Fang, Y. et al. 2021, “Role of Epoxide Hydrolases and Cytochrome P450s on Metabolism of KZR-616, a First-in-Class Selective Inhibitor of the Immunoproteasome,” Drug Metabolism and Disposition, September 2021, 49 (9) 810-821. In mouse models of rheumatoid arthritis and systemic lupus erythematosus (SLE), KZR-616 blocked disease progression at well tolerated doses without affecting normal T-cell-dependent immune responses. See id. KZR-616 is currently being evaluated in phase 2 clinical trials in patients with SLE and lupus nephritis. See id.

Additionally, the selective LMP2 inhibitors UK-101 and IPSI-001 have been shown to exhibit antitumor activity against multiple myelomas. See, e.g., id. Similarly, PR-924, a selective inhibitor of LMP7, inhibited growth and triggered apoptosis in multiple myeloma (MM) cell lines and primary patient MM cells, without significantly affecting normal peripheral blood mononuclear cells. See Singh, A. V. et al. 2011, “PR-924, a selective inhibitor of LMP7, blocks multiple myeloma cell growth both in vitro and in vivo,” Br. J. Haematol, 152 (2): 155-63.

As disclosed in WO 2009/154737 and elsewhere, certain boronic acid-based compounds have been disclosed as effective immunoproteasome inhibitors. Despite these promising candidates, there exists a need for additional boronic acid-based immunoproteasome inhibitors, particularly because it is known that boronic acids can be amorphous and difficult to manufacture on large scale or update the purity of the final product. Additionally, certain boronic acids are prone to hydrolysis and oxidation due to the presence of the boronic acid functional groups. This inherent chemical instability may lead to potential chemical instability during drug substance and drug product manufacturing and storage. Accordingly, a need exists for additional compounds that exhibit similar properties in vivo but may be more easily or efficiently manufactured and/or stored. Preparation of pharmaceutical salts of compounds of interest are a common strategy for attempting to find a more stable compound for manufacturing and storage. It was surprisingly found that, when attempting to make acid salts of certain boronic acid immunoproteasome inhibitor compounds, cyclic boronic esters formed. Further, some of these esters have been shown to be more stable than the corresponding acid, have greater crystallinity or both.

Accordingly, in one aspect, provided herein cyclic boronic esters and salts thereof for use in treating conditions associated with LMP2 and/or LMP7.

### SUMMARY OF THE DISCLOSURE

Described herein, in certain embodiments, are cyclic boronic esters useful as immunoproteasome inhibitors.

Embodiment 1. A compound of Formula (I), or a pharmaceutically acceptable salt thereof,

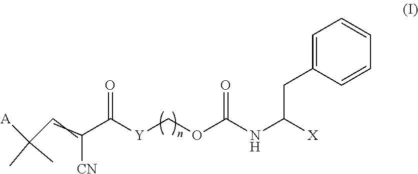

wherein
A is an optionally substituted mono- or bicyclic heterocyclic ring containing at least one nitrogen atom bond to the methylene unit;
Y is an optionally substituted mono- or bicyclic heterocyclic ring containing at least one nitrogen atom bonded to the carbonyl group;
X is a boronic ester, which is bound to the methylene unit through the boron atom and is chosen from a citric acid ester, a pinacol ester, a malic acid ester, a salicylic acid ester, a lactic acid ester, and a tartaric acid ester; and
n is 0 or 1.

Embodiment 2. The compound according to embodiment 1, wherein A is an optionally substituted mono- or bicyclic 5 or 6-membered heterocyclic ring.

Embodiment 3. The compound according to any one of the preceding embodiments, wherein A is chosen from an optionally substituted pyrrolidinyl and an optionally substituted morpholinyl.

Embodiment 4. The compound according to any one of the preceding embodiments, wherein A is chosen from:

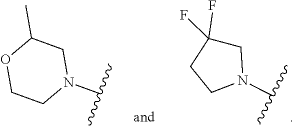

Embodiment 5. The compound according to any one of the preceding embodiments, wherein Y is an optionally substituted mono- or bicyclic 6 or 7-membered heterocyclic ring.

Embodiment 6. The compound according to any one of the preceding embodiments, wherein Y is chosen from piperidinyl, azepanyl, and 7-azabicyclo[2.2.1]heptan-7-yl, each of which is optionally substituted.

Embodiment 7. The compound according to any one of the preceding embodiments, wherein Y is chosen from:

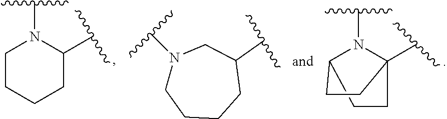 [SMILES: *C1CCCCN1* ; *C1CCCCN(*)C1 ; *N1C2CCC1(*)CC2]

Embodiment 8. The compound according to any one of the preceding embodiments, wherein X is a citric acid boronic ester.

Embodiment 9. The compound according to any one of the preceding embodiments, wherein X is chosen from:

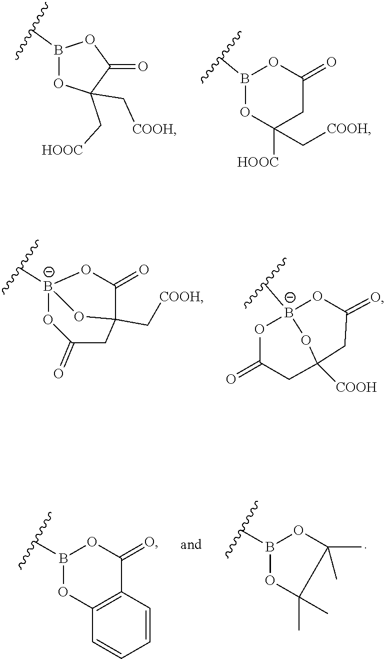

Embodiment 10. A compound prepared by reacting a compound of Formula (i) or a pharmaceutically acceptable salt thereof with a reagent chosen from citric acid, malic acid, salicylic acid, lactic acid, tartaric acid and pinacol, wherein Formula (i) is:

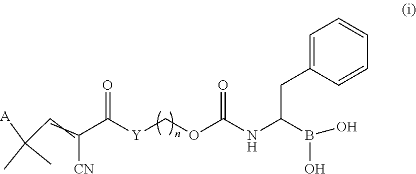

wherein,
A is an optionally substituted mono- or bicyclic heterocyclic ring containing at least one nitrogen atom bond to the methylene unit;
Y is an optionally substituted mono- or bicyclic heterocyclic ring containing at least one nitrogen atom bonded to the carbonyl group; and
n is 0 or 1.

Embodiment 11. The compound according to embodiment 10, wherein A is an optionally substituted mono- or bicyclic 5 or 6-membered heterocyclic ring.

Embodiment 12. The compound according to embodiment 10 or 11, wherein A is chosen from:

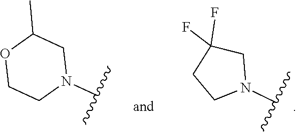

Embodiment 13. The compound according to any one of embodiments 10-12, wherein Y is an optionally substituted mono- or bicyclic 6 or 7-membered heterocyclic ring.

Embodiment 14. The compound according to any one of embodiments 10-13, wherein Y is chosen from

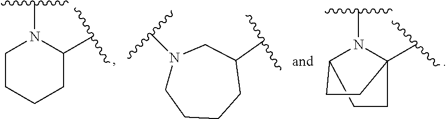 [SMILES: *C1CCCCN1* ; *C1CCCCN(*)C1 ; *N1C2CCC1(*)CC2]

Embodiment 15. The compound according to any one of embodiments 10-14, wherein the reagent is citric acid.

Embodiment 16. A compound chosen from:

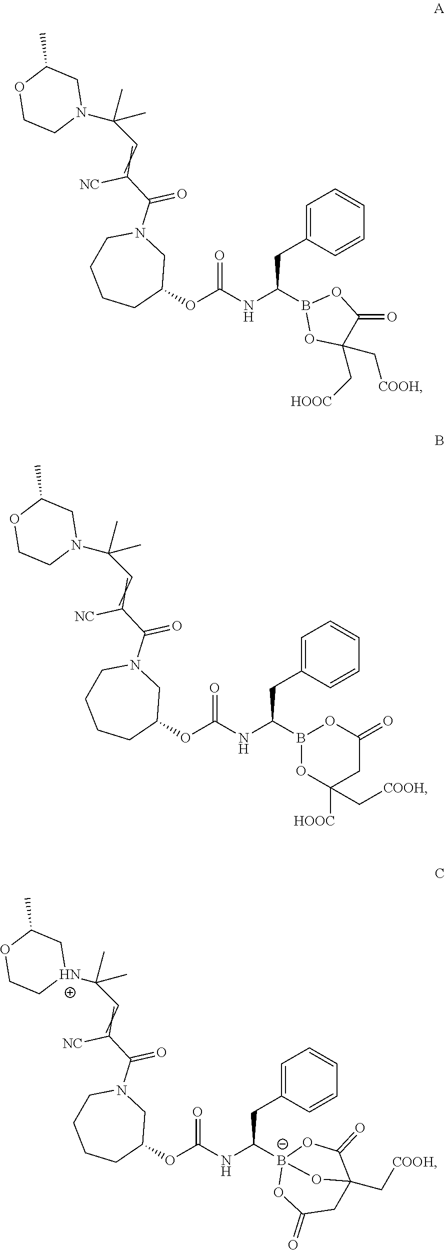 [SMILES: C[C@@H]1CN(C(C)(C)C=C(C#N)C(=O)N2CCCC[C@@H](OC(=O)N[C@@H](Cc3ccccc3)B3OC(=O)C(CC(=O)O)(CC(=O)O)O3)C2)CCO1 ; C[C@@H]1CN(C(C)(C)C=C(C#N)C(=O)N2CCCC[C@@H](OC(=O)N[C@@H](Cc3ccccc3)B3OC(=O)CC(CC(=O)O)(C(=O)O)O3)C2)CCO1 ; C[C@@H]1C[NH+](C(C)(C)C=C(C#N)C(=O)N2CCCC[C@@H](OC(=O)N[C@@H](Cc3ccccc3)[B-]34OC(=O)CC(CC(=O)O)(O3)C(=O)O4)C2)CCO1]

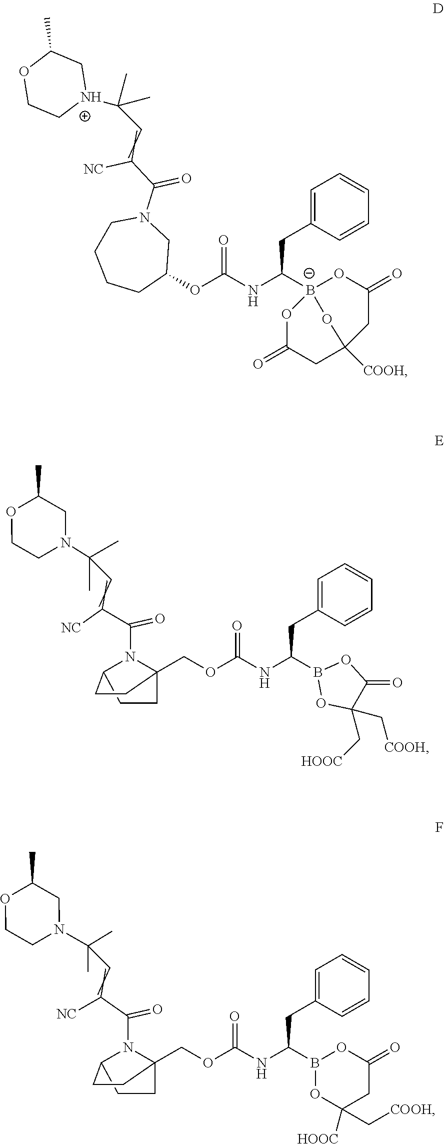 [SMILES: C[C@@H]1C[NH+](C(C)(C)C=C(C#N)C(=O)N2CCCC[C@@H](OC(=O)N[C@@H](Cc3ccccc3)[B-]34OC(=O)CC(C(=O)O)(CC(=O)O3)O4)C2)CCO1 ; C[C@H]1CN(C(C)(C)C=C(C#N)C(=O)N2C3CCC2(COC(=O)N[C@@H](Cc2ccccc2)B2OC(=O)C(CC(=O)O)(CC(=O)O)O2)CC3)CCO1 ; C[C@H]1CN(C(C)(C)C=C(C#N)C(=O)N2C3CCC2(COC(=O)N[C@@H](Cc2ccccc2)B2OC(=O)CC(CC(=O)O)(C(=O)O)O2)CC3)CCO1]

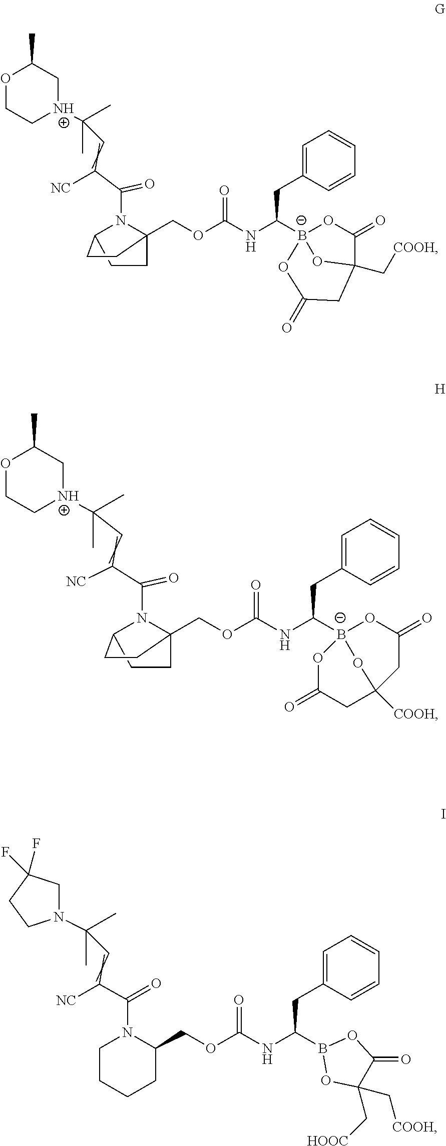 [SMILES: C[C@H]1C[NH+](C(C)(C)C=C(C#N)C(=O)N2C3CCC2(COC(=O)N[C@@H](Cc2ccccc2)[B-]24OC(=O)CC(CC(=O)O)(O2)C(=O)O4)CC3)CCO1 ; C[C@H]1C[NH+](C(C)(C)C=C(C#N)C(=O)N2C3CCC2(COC(=O)N[C@@H](Cc2ccccc2)[B-]24OC(=O)CC(C(=O)O)(CC(=O)O2)O4)CC3)CCO1 ; CC(C)(C=C(C#N)C(=O)N1CCCC[C@@H]1COC(=O)N[C@@H](Cc1ccccc1)B1OC(=O)C(CC(=O)O)(CC(=O)O)O1)N1CCC(F)(F)C1]

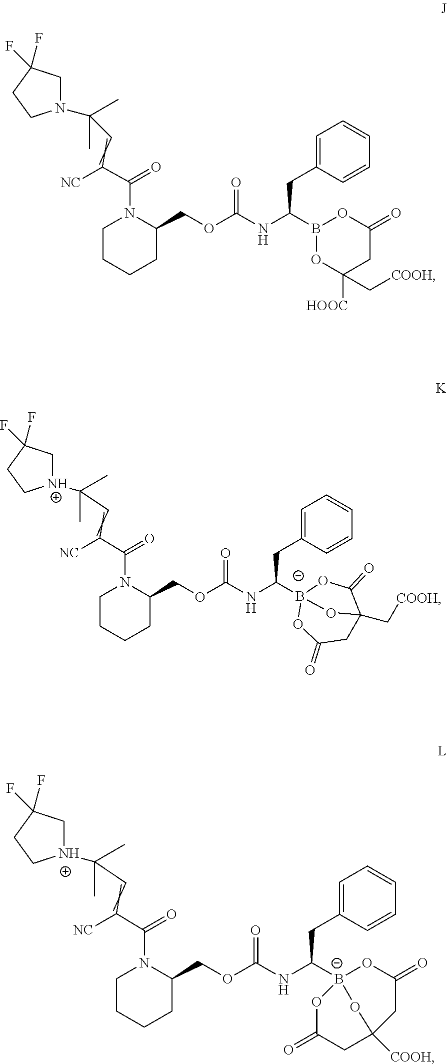 [SMILES: CC(C)(C=C(C#N)C(=O)N1CCCC[C@@H]1COC(=O)N[C@@H](Cc1ccccc1)B1OC(=O)CC(CC(=O)O)(C(=O)O)O1)N1CCC(F)(F)C1 ; CC(C)(C=C(C#N)C(=O)N1CCCC[C@@H]1COC(=O)N[C@@H](Cc1ccccc1)[B-]12OC(=O)CC(CC(=O)O)(O1)C(=O)O2)[NH+]1CCC(F)(F)C1 ; CC(C)(C=C(C#N)C(=O)N1CCCC[C@@H]1COC(=O)N[C@@H](Cc1ccccc1)[B-]12OC(=O)CC(C(=O)O)(CC(=O)O1)O2)[NH+]1CCC(F)(F)C1]

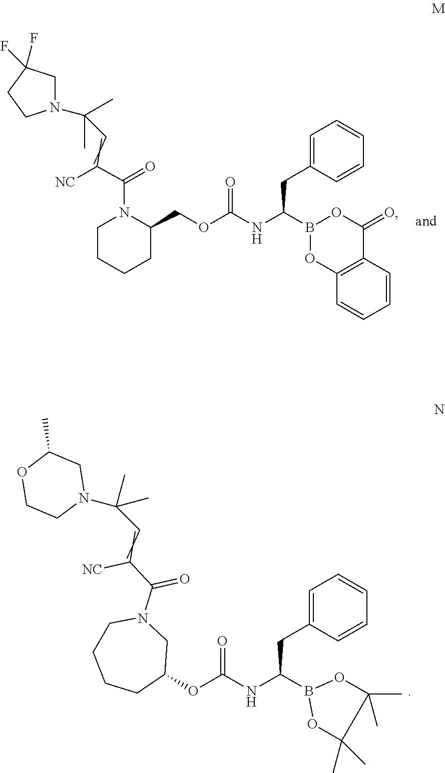 [SMILES: CC(C)(C=C(C#N)C(=O)N1CCCC[C@@H]1COC(=O)N[C@@H](Cc1ccccc1)B1OC(=O)c2ccccc2O1)N1CCC(F)(F)C1 ; C[C@@H]1CN(C(C)(C)C=C(C#N)C(=O)N2CCCC[C@@H](OC(=O)N[C@@H](Cc3ccccc3)B3OC(C)(C)C(C)(C)O3)C2)CCO1]

Embodiment 17. A compound according to embodiment 16 chosen from

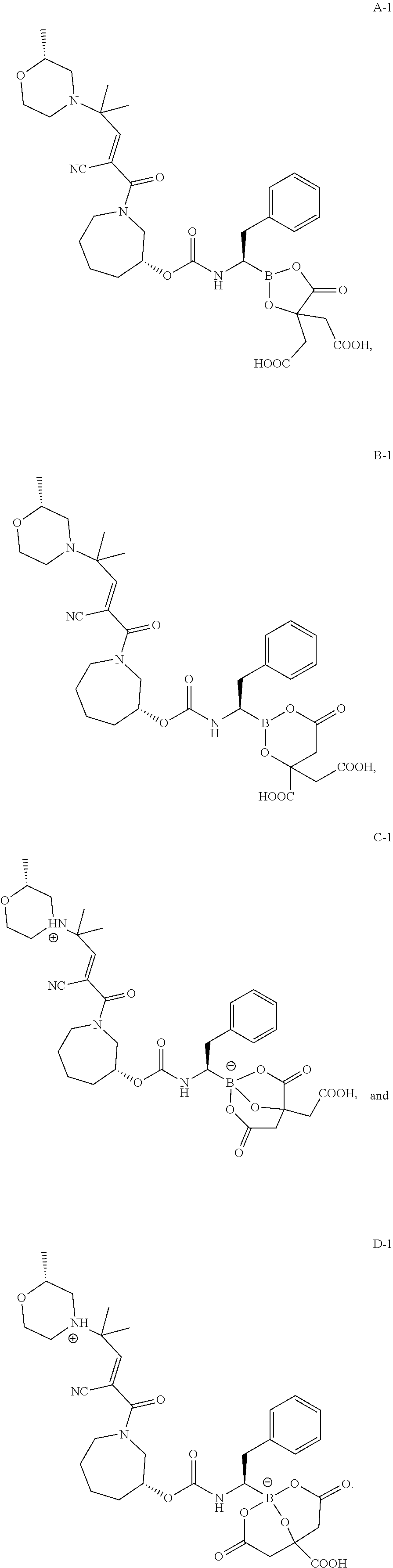 [SMILES: C[C@@H]1CN(C(C)(C)/C=C(\C#N)C(=O)N2CCCC[C@@H](OC(=O)N[C@@H](Cc3ccccc3)B3OC(=O)C(CC(=O)O)(CC(=O)O)O3)C2)CCO1 ; C[C@@H]1CN(C(C)(C)/C=C(\C#N)C(=O)N2CCCC[C@@H](OC(=O)N[C@@H](Cc3ccccc3)B3OC(=O)CC(CC(=O)O)(C(=O)O)O3)C2)CCO1 ; C[C@@H]1C[NH+](C(C)(C)/C=C(\C#N)C(=O)N2CCCC[C@@H](OC(=O)N[C@@H](Cc3ccccc3)[B-]34OC(=O)CC(CC(=O)O)(O3)C(=O)O4)C2)CCO1 ; C[C@@H]1C[NH+](C(C)(C)/C=C(\C#N)C(=O)N2CCCC[C@@H](OC(=O)N[C@@H](Cc3ccccc3)[B-]34OC(=O)CC(C(=O)O)(CC(=O)O3)O4)C2)CCO1]

Embodiment 18. A boronic acid ester of ((R)-1-(((((R)-1-(2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester is chosen from a citric acid ester, a pinacol ester, a malic acid ester, a salicylic acid ester, a lactic acid ester, and a tartaric acid ester.

Embodiment 19. A boronic acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester is chosen from a citric acid ester, a pinacol ester, a malic acid ester, a salicylic acid ester, a lactic acid ester, and a tartaric acid ester.

Embodiment 20. A boronic ester of [(1R)-1-[([7-(2-cyano-2-[2-methyl-2-[(2S)-2-methylmorpholin-4-yl]propylidene]acetyl)-7-azabicyclo[2.2.1]heptan-1-yl]methoxy]carbonyl)amino]-2-phenylethyl]boronic acid or a pharmaceutically acceptable salt thereof, wherein the ester is chosen from a citric acid ester, a pinacol ester, a malic acid ester, a salicylic acid ester, a lactic acid ester, and a tartaric acid ester.

Embodiment 21. A boronic ester of ((R)-1-(((((R)-1-(2-cyano-4-(3,3-difluoropyrrolidin-1-yl)-4-methylpent-2-enoyl)piperidin-2-yl)methoxy)carbonyl)amino)-2-phenylethyl)boronic acid or a pharmaceutically acceptable salt thereof, wherein the ester is chosen from a citric acid ester, a pinacol ester, a malic acid ester, a salicylic acid ester, a lactic acid ester, and a tartaric acid ester.

Embodiment 22. The boronic ester according to any one of embodiments 18-21, wherein the ester is a citric acid ester.

Embodiment 23. A boronic acid ester of ((R)-1-(((((R)-1-(2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester converts to its corresponding acid upon exposure to physiological conditions.

Embodiment 24. A boronic acid ester of ((R)-1-(((((R)-1-(2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester is stable at 40° C. and 75% relative humidity for at least three weeks.

Embodiment 25. A boronic ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester a citric acid ester and is at least about 95% pure.

Embodiment 26. The boronic ester of embodiment 25, wherein the ester is at least about 99% pure.

Embodiment 27. A crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid, wherein the crystalline form is Crystalline Form I, characterized by an X-ray powder diffractogram comprising at least three peaks chosen from peaks at about 6.6±0.2, 11.2±0.2, 13.2±0.2, 13.8±0.2, 14.3±0.2, 15.6±0.2, 16.8±0.2, 17.5±0.2, 18.5±0.2, and 19.0±0.2 2-θ.

Embodiment 28. The crystalline Form I according to embodiment 27, characterized by an X-ray powder diffractogram substantially similar to that in FIG. 6.

Embodiment 29. The crystalline Form I according to embodiment 27 or 28, characterized by a DSC thermogram having a peak endotherm at about 192.7° C.

Embodiment 30. The crystalline Form I according to any one of embodiments 27-29, characterized by a DSC thermogram showing onset of melting at about 179.5° C.

Embodiment 31. The crystalline Form I according to any one of embodiments 27-30, characterized by a DSC thermogram substantially similar to that in FIG. 7.

Embodiment 32. The crystalline Form I according to any one of embodiments 27-31, characterized by a mass loss of less than about 0.5 wt. % between about 25° C. and about 150° C. by thermogravimetric analysis.

Embodiment 33. The crystalline Form I according to any one of embodiments 27-32, characterized by a TGA thermogram substantially similar to that in FIG. 7.

Embodiment 34. A crystalline form of a citric acid ester of (I-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid, wherein the crystalline form is Crystalline Form II, characterized by an X-ray powder diffractogram comprising at least three peaks chosen from peaks at about from 6.1±0.2, 8.1±0.2, 10.6±0.2, 11.0±0.2, 11.9±0.2, 14.0±0.2, 14.7±0.2, 16.6±0.2, 43.7±0.2, and 18.4±0.2 2-θ.

Embodiment 35. The crystalline Form II according to embodiment 34, characterized by an X-ray powder diffractogram substantially similar to that in FIG. 8.

Embodiment 36. The crystalline Form II according to embodiment 34 or 35, characterized by a DSC thermogram having a peak endotherm at about 173.7° C.

Embodiment 37. The crystalline Form II according to any one of embodiments 34-36, characterized by a DSC thermogram showing onset of melting at about 164.9° C.

Embodiment 38. The crystalline Form II according to any one of embodiments 34-37, characterized by a DSC thermogram substantially similar to that in FIG. 9.

Embodiment 39. The crystalline Form II according to any one of embodiments 34-38, characterized by a mass loss of about 5.6 wt. % between about 25° C. and about 110° C. by thermogravimetric analysis.

Embodiment 40. The crystalline Form II according to any one of embodiments 34-39, characterized by a TGA thermogram substantially similar to that in FIG. 9.

Embodiment 41. The crystalline Form I according to any one of embodiments 27-33 prepared by contacting crystalline Form II according to any one of embodiments 34-40 with an alcohol or an alcohol mixture.

Embodiment 42. A crystalline form a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid, wherein the crystalline form is Crystalline Form III, characterized by an X-ray powder diffractogram comprising at least three peaks chosen from peaks at about 5.9±0.2, 6.9±0.2, 9.4±0.2, 10.5±0.2, 11.1±0.2, 12.3±0.2, 12.7±0.2, 13.8±0.2, 14.3±0.2, and 15.2±0.2 2-θ.

Embodiment 43. The crystalline Form III according to embodiment 42, characterized by an X-ray powder diffractogram substantially similar to that in FIG. 10.

Embodiment 44. The crystalline Form III according to embodiment 42 or 43, characterized by a DSC thermogram having a peak endotherm at about 176.5° C.

Embodiment 45. The crystalline Form III according to any one of embodiments 42-44, characterized by a DSC thermogram having a broad endotherm from about 37.9° C. to about 100° C.

Embodiment 46. The crystalline Form III according to embodiment 45, wherein said broad endotherm has a peak at about 70.4° C.

Embodiment 47. The crystalline Form III according to any one of embodiments 42-46, characterized by a DSC thermogram substantially similar to that in FIG. 11.

Embodiment 48. The crystalline Form III according to any one of embodiments 42-47, characterized by a mass loss of about 3.1 wt. % between about 25° C. and about 105° C. by thermogravimetric analysis.

Embodiment 49. The crystalline Form III according to any one of embodiments 42-48, characterized by a TGA thermogram substantially similar to that in FIG. 11.

Embodiment 50. The crystalline Form III according to any one of embodiments 42-49 prepared by a process comprising contacting an alkyl ester, ether and/or toluene with Crystalline Form II according to any one of embodiments 34-40.

Embodiment 51. The crystalline Form III prepared by the process according to embodiment 50, wherein the alkyl ester is isopropyl acetate.

Embodiment 52. The crystalline Form III prepared by the process according to embodiment 50 or 51, wherein the process further comprises isolating Form III from the alkyl ester.

Embodiment 53. A pharmaceutical composition comprising at least one compound according to any one of embodiments 1-17 and a pharmaceutically acceptable excipient.

Embodiment 54. A pharmaceutical composition comprising at least one boronic ester according to any one of embodiments 18-26 and a pharmaceutically acceptable excipient.

Embodiment 55. A pharmaceutical composition comprising at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid according to any one of embodiments 27-52 and a pharmaceutically acceptable excipient.

Embodiment 56. A method of inhibiting Large Multifunctional Protease 2 (LMP2) in a subject comprising administering to said subject in need of said inhibition a therapeutically effective amount of a compound of any one of embodiments 1-17, at least one boronic ester according to any one of embodiments 18-26 or at least one crystalline form of a citric acid ester of ((R)-1-(((((R)-1-((E)-2-cyano-4-methyl-4-((R)-2-methylmorpholino)pent-2-enoyl)azepan-3 -yl)oxy)carbonyl)amino)-2-phenylethyl)boronic acid according to any one of embodiments 27-52.

Embodiment 57. A method of inhibiting Large Multifunctional Protease 7 (LMP7) in a subject comprising administering to said subject in need of said inhibition a therapeutically effective amount of a compound of any one of embodiments 1-17, at least one boronic ester according to any one of embodiments 18-26 or at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid according to any one of embodiments 27-52.

Embodiment 58. A method of treating a disease chosen from an autoimmune disorder, an inflammatory disorder, and a hematological disorder in a patient in need of such treatment, comprising administering to the patient a therapeutically effective amount of a compound of any one of embodiments 1-17, at least one boronic ester according to any one of embodiments 18-26 or at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid according to any one of embodiments 27-52.

Embodiment 59. The method of embodiment 58, wherein the disease is chosen from lupus, arthritis including rheumatoid arthritis and psoriatic arthritis, scleroderma, ankylosing spondylitis, Duchene muscular dystrophy (DMD), Becker muscular dystrophy (BMD), idiopathic inflammatory myopathies (IIMs), polymyositis, sporadic inclusion body myositis, dermatomyositis, immune-mediated necrotizing myopathies (IMNM), psoriasis, multiple sclerosis, inflammatory bowel disease, Behçet's disease, ulcerative colitis, Crohn's disease, Sjogren's Syndrome, bronchitis, conjunctivitis, pancreatitis, cholecystitis, bronchiectasis, aortic valve stenosis, restenosis, fibrosis, infection, ischemia, cardiovascular disease, hepatitis, cirrhosis, steatohepatitis, liver inflammation, Alzheimer's Disease (AD), amyotrophic lateral sclerosis (ALS), Huntington's disease, body myositis, myofibrillar myopathy, Graft-versus-Host Disease (GVHD), and multiple myeloma.

Embodiment 60. Use of a composition as a medicament for inhibiting Large Multifunctional Protease 2 (LMP2) in a subject, wherein the composition comprises a therapeutically effective amount of a compound of any one of embodiments 1-17, at least one boronic ester according to any one of embodiments 18-26 or at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid according to any one of embodiments 27-52.

Embodiment 61. Use of a composition as a medicament for inhibiting Large Multifunctional Protease 7 (LMP7) in a subject, wherein the composition comprises a therapeutically effective amount of a compound of any one of embodiments 1-17, at least one boronic ester according to any one of embodiments 18-26 or at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid according to any one of embodiments 27-52.

Embodiment 62. Use of a composition as a medicament for treating a disease in a subject in need therefore, wherein the composition comprises administering to the patient a therapeutically effective amount of a compound of any one of embodiments 1-17, at least one boronic ester according to any one of embodiments 18-26 or at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid according to any one of embodiments 27-52; and wherein the disease chosen from an autoimmune disorder, an inflammatory disorder, and a hematological disorder.

Embodiment 63. The use according to embodiment 62, wherein the disease is chosen from lupus, arthritis including rheumatoid arthritis and psoriatic arthritis, scleroderma, ankylosing spondylitis, Duchene muscular dystrophy (DMD), Becker muscular dystrophy (BMD), idiopathic inflammatory myopathies (IIMs), polymyositis, sporadic inclusion body myositis, dermatomyositis, immune-mediated necrotizing myopathies (IMNM), psoriasis, multiple sclerosis, inflammatory bowel disease, Behçet's disease, ulcerative colitis, Crohn's disease, Sjogren's Syndrome, bronchitis, conjunctivitis, pancreatitis, cholecystitis, bronchiectasis, aortic valve stenosis, restenosis, fibrosis, infection, ischemia, cardiovascular disease, hepatitis, cirrhosis, steatohepatitis, liver inflammation, Alzheimer's Disease (AD), amyotrophic lateral sclerosis (ALS), Huntington's disease, body myositis, myofibrillar myopathy, Graft-versus-Host Disease (GVHD), and multiple myeloma.

### BRIEF DESCRIPTION OF THE DRAWINGS

FIG. 1 provide an exemplary NMR spectrum of the pinacol ester of ((R)-1-(((((R)-1-(2-cyano-4-methyl-4-((R)-2-methylmorpholino)pent-2-enoyl)azepan-3-yl)oxy)carbonyl)amino)-2-phenylethyl)boronic acid.

FIG. 2 provides an NMR spectrum of the citric acid esters of [(1R)-1-[([[7-(2-cyano-2-[2-methyl-2-[(2S)-2-methylmorpholin-4-yl]propylidene]acetyl)-7-azabicyclo[2.2.1]heptan-1-yl]methoxy]carbonyl)amino]-2-phenylethyl]boronic acid.

FIG. 3 provides an NMR spectrum of Boronic Acid 3 from Example 5.

FIG. 4 provides an NMR spectrum of the citric acid esters of ((R)-1-(((((R)-1-(2-cyano-4-(3,3-difluoropyrrolidin-1-yl)-4-methylpent-2-enoyl)piperidin-2-yl)methoxy)carbonyl)amino)-2-phenylethyl)boronic acid.

FIG. 5 provides an NMR spectrum of the salicylic acid ester of ((R)-1-(((((R)-1-(2-cyano-4-(3,3-difluoropyrrolidin-1-yl)-4-methylpent-2-enoyl)piperidin-2-yl)methoxy)carbonyl)amino)-2-phenylethyl)boronic acid.

FIG. 6 provides an X-ray powder diffractogram of Form I of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid.

FIG. 7 provides a TGA thermogram and a DSC thermogram of Form I of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid.

FIG. 8 provides an X-ray powder diffractogram of Form II of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid.

FIG. 9 provides a TGA thermogram and a DSC thermogram of Form II of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid.

FIG. 10 provides an X-ray powder diffractogram of Form III of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid.

FIG. 11 provides a TGA thermogram and a DSC thermogram of Form III of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid.

FIG. 12 provides an X-ray powder diffractogram of Form IV of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid.

FIG. 13 provides a TGA thermogram and a DSC thermogram of Form IV of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid.

FIG. 14 provides an X-ray powder diffractogram of Form V of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid.

FIG. 15 provides a TGA thermogram and a DSC thermogram of Form V of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid.

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless the context requires otherwise. Furthermore, use of the term “including” as well as other forms, such as “include”, “includes,” and “included,” is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

“Alkyl” refers to an unbranched or branched saturated hydrocarbon chain. As used herein, alkyl has 1 to 20 carbon atoms (i.e., C₁-C₂₀ alkyl), 1 to 10 carbon atoms (i.e., C₁-C₁₀ alkyl), 1 to 6 carbon atoms (i.e., C₁-C₆ alkyl) or 1 to 3 carbon atoms (i.e., C₁-C₃ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), isobutyl (i.e., —CH₂CH(CH₃)₂), sec-butyl (i.e., —CH(CH₃)CH₂CH₃), and tert-butyl (i.e., —C(CH₃)₃); and “propyl” includes n-propyl (i.e., —(CH₂)₂CH₃) and isopropyl (i.e., —CH(CH₃)₂).

“Methylene” refers to a —CH₂— group.

“Carbonyl” refers to —C(═O)— group.

The terms “heterocyclyl” or “heterocyclic ring” refer to a saturated or unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more oxo (C═O) or N-oxide (N—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 1 to 10 ring carbon atoms, 1 to 8 carbon atoms, 1 to 6 carbon atoms, or 1 to 4 carbon atoms. As used herein, heterocyclyl has 1 to 5 ring heteroatoms, 1 to 4 heteroatoms, 1 to 3 heteroatoms, or 1 to 2 heteroatoms independently selected from nitrogen, sulfur and oxygen. Examples of heterocyclyl groups include azepanyl, 7-azabicyclo[2.2.1]heptan-7-yl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl and 1,1-dioxo-thiomorpholinyl.

As used herein, the term “optionally substituted” when referring to a heterocyclic group refers to a heterocyclic group that has one or more hydrogen atoms on the carbon or on a heteroatom replaced with an alkyl group, halo, cyano or hydroxy.

“Cyano” refers to the group-CN.

“Halogen” or “halo” includes fluoro, chloro, bromo, and iodo.

“Hydroxy” refers to the group-OH.

Certain commonly used alternative chemical names may be used. For example, a divalent group such as a divalent “alkyl” group, a divalent “phenyl” group, a divalent “heteroaryl” group, a divalent “heterocyclyl” group etc., may also be referred to as an “alkylene” group, an “phenylene” group, a “heteroarylene” group, or a “heterocyclylene” group, respectively.

The terms “optional” or “optionally” means that the subsequently described event or circumstance may or may not occur and that the description includes instances where said event or circumstance occurs and instances in which it does not.

“Pharmaceutically acceptable” refers to compounds, salts, compositions, dosage forms, and other materials which are useful in preparing a pharmaceutical composition that is suitable for human pharmaceutical use.

The term “pharmaceutically acceptable salt” of a given compound refers to salts that retain the biological effectiveness and properties of the given compound and which are not biologically or otherwise undesirable.

The compounds described herein, or their pharmaceutically acceptable salts, may include an asymmetric center and may thus give rise to enantiomers, diastereomers, and other stereoisomeric forms that may be defined, in terms of absolute stereochemistry, as (R)- or (S)- or, as (D)- or (L)- for amino acids. The disclosure is meant to include all such possible isomers, as well as their racemic and optically pure forms. Optically active (±) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

“Tautomer” refers to alternate forms of a compound that differ in the position of a proton, such as enol-keto and imine-enamine tautomers, or the tautomeric forms of heteroaryl groups containing a ring atom attached to both a ring-NH-moiety and a ring=N moiety such as pyrazoles, imidazoles, benzimidazoles, triazoles, and tetrazoles. All tautomeric forms of the compounds described herein are intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

“Diastereoisomers” are stereoisomers that have at least two asymmetric atoms, but which are not mirror-images of each other.

As used herein, “pharmaceutically acceptable carrier” or “pharmaceutically acceptable excipient” or “excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Effective amount” or dose of a compound or a composition refers to that amount of the compound or the composition that results in an intended result. Effective amounts can be determined by standard pharmaceutical procedures in cell cultures or experimental animals including, without limitation, by determining the LD₅₀ (the dose lethal to 50% of the population) and the ED₅₀ (the dose therapeutically effective in 50% of the population).

“Therapeutically effective amount” or dose of a compound or a composition refers to that amount of the compound or the composition that results in reduction or inhibition of symptoms or a prolongation of survival in a subject (i.e., a human patient). The results may require multiple doses of the compound or the composition.

“Treating” or “treatment” of a disease in a subject refers to 1) preventing the disease from occurring in a patient that is predisposed or does not yet display symptoms of the disease; 2) inhibiting the disease or arresting its development; or 3) ameliorating or causing regression of the disease. As used herein, “treatment” or “treating” is an approach for obtaining beneficial or desired results including clinical results. For the purposes of this disclosures, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a subject. Also encompassed by “treatment” is a reduction of pathological consequence of the disease or disorder. The methods of the invention contemplate any one or more of these aspects of treatment.

As used herein, the terms “subject(s)” and “patient(s)” mean any mammal. In some embodiments, the mammal is a human. None of the terms require or are limited to situations characterized by the supervision (e.g., constant or intermittent) of a health care worker (e.g., a doctor, a registered nurse, a nurse practitioner, a physician's assistant, an orderly or a hospice worker).

As used herein, the terms “pharmaceutical composition” or “medicament” refer to a composition suitable for pharmaceutical use in a subject.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein is a compound of Formula (I), or a pharmaceutically acceptable salt thereof:

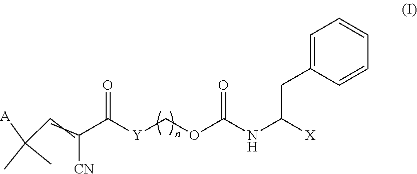

wherein
A is an optionally substituted mono- or bicyclic heterocyclic ring containing at least one nitrogen atom bond to the methylene unit;
Y is an optionally substituted mono- or bicyclic heterocyclic ring containing at least one nitrogen atom bonded to the carbonyl group;
X is a boronic ester, which is bound to the methylene unit through the boron atom and is chosen from a citric acid ester, a pinacol ester, a malic acid ester, a salicylic acid ester, a lactic acid ester, and a tartaric acid ester; and
   n is 0 or 1.

In some embodiments, A is an optionally substituted mono- or bicyclic 5 or 6-membered heterocyclic ring. In some embodiments, A is a mono- or bicyclic 5 or 6-membered heterocyclic ring optionally substituted with at least one alkyl and/or halo. In some embodiments, A is a monocyclic 5 or 6-membered heterocyclic ring optionally substituted with at least one alkyl and/or halo. In some embodiments, A is chosen from an optionally substituted pyrrolidinyl and an optionally substituted morpholinyl. In some embodiments, A is a pyrrolidinyl or morpholinyl optionally substituted with at least one alkyl and/or halo. In some embodiments, A is chosen from:

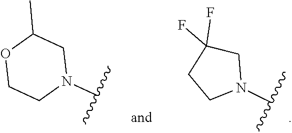

In some embodiments, A is

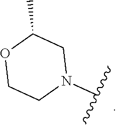 [SMILES: *N1CCO[C@H](C)C1]

In some embodiments, Y is an optionally substituted mono- or bicyclic 6 or 7-membered heterocyclic ring. In some embodiments, Y is an optionally substituted mono- or bicyclic 7-membered heterocyclic ring. In some embodiments, Y is a mono- or bicyclic 6 or 7-membered heterocyclic ring optionally substituted with at least one alkyl and/or halo. In some embodiments, Y is a mono- or bicyclic 6 or 7-membered heterocyclic ring is unsubstituted. In some embodiments, Y is chosen from piperidinyl, azepanyl, and 7-azabicyclo[2.2.1]heptan-7-yl, each of which is optionally substituted. In some embodiments, Y is chosen from piperidinyl, azepanyl, and 7-azabicyclo[2.2.1]heptan-7-yl, each of which are unsubstituted. In some embodiments, Y is chosen from:

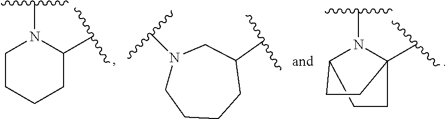 [SMILES: *C1CCCCN1* ; *C1CCCCN(*)C1 ; *N1C2CCC1(*)CC2]

In some embodiments, X is chosen from a citric acid ester and a pinacol ester. In some embodiments, X is a citric acid boronic ester. In some embodiments, X is chosen from:

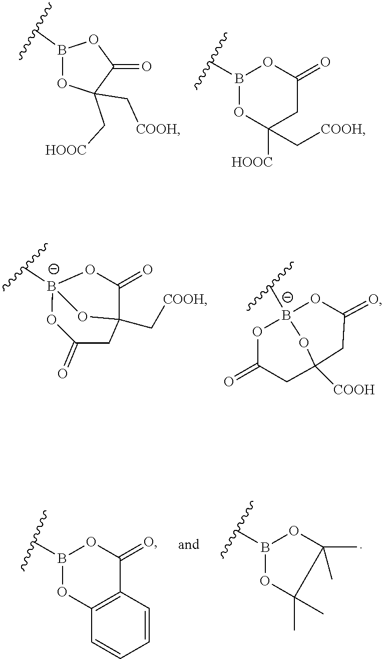

In some embodiments, X is chosen from:

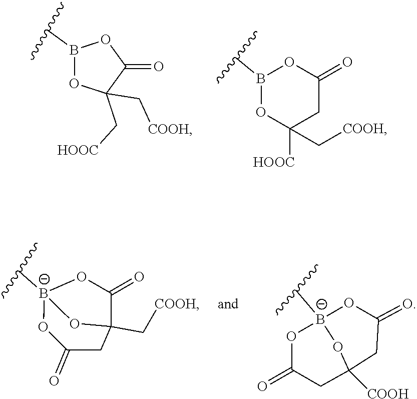 [SMILES: *B1OC(=O)C(CC(=O)O)(CC(=O)O)O1 ; *B1OC(=O)CC(CC(=O)O)(C(=O)O)O1 ; *[B-]12OC(=O)CC(CC(=O)O)(O1)C(=O)O2 ; *[B-]12OC(=O)CC(C(=O)O)(CC(=O)O1)O2]

In some embodiments, X is

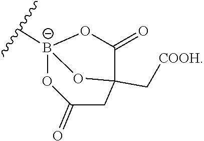 [SMILES: *[B-]12OC(=O)CC(CC(=O)O)(O1)C(=O)O2]

In some embodiments, n is 0. In some embodiments, n is 1.

In one aspect, provided herein is a compound prepared by reacting a compound of Formula (i) or a pharmaceutically acceptable salt thereof with a reagent chosen from citric acid, malic acid, salicylic acid, lactic acid, tartaric acid and pinacol, wherein Formula (i) is

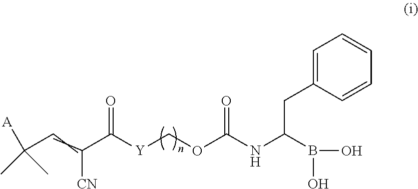

wherein,
A is an optionally substituted mono- or bicyclic heterocyclic ring containing at least one nitrogen atom bond to the methylene unit;
Y is an optionally substituted mono- or bicyclic heterocyclic ring containing at least one nitrogen atom bonded to the carbonyl group; and
   n is 0 or 1.

In some embodiments, A is an optionally substituted mono- or bicyclic 5 or 6-membered heterocyclic ring. In some embodiments, A is a mono- or bicyclic 5 or 6-membered heterocyclic ring optionally substituted with at least one alkyl and/or halo. In some embodiments, A is a monocyclic 5 or 6-membered heterocyclic ring optionally substituted with at least one alkyl and/or halo. In some embodiments, A is chosen from an optionally substituted pyrrolidinyl and an optionally substituted morpholinyl. In some embodiments, A is a pyrrolidinyl or morpholinyl optionally substituted with at least one alkyl and/or halo. In some embodiments, A is chosen from:

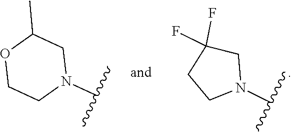

In some embodiments, A is

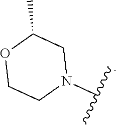 [SMILES: *N1CCO[C@H](C)C1]

In some embodiments, Y is an optionally substituted mono- or bicyclic 6 or 7-membered heterocyclic ring. In some embodiments, Y is an optionally substituted mono- or bicyclic 7-membered heterocyclic ring. In some embodiments, Y is a mono- or bicyclic 6 or 7-membered heterocyclic ring optionally substituted with at least one alkyl and/or halo. In some embodiments, Y is a mono- or bicyclic 6 or 7-membered heterocyclic ring is unsubstituted. In some embodiments, Y is chosen from piperidinyl, azepanyl, and 7-azabicyclo[2.2.1]heptan-7-yl, each of which is optionally substituted. In some embodiments, Y is chosen from piperidinyl, azepanyl, and 7-azabicyclo[2.2.1]heptan-7-yl, each of which are unsubstituted. In some embodiments, Y is chosen from:

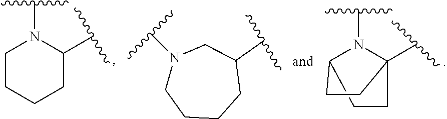 [SMILES: *C1CCCCN1* ; *C1CCCCN(*)C1 ; *N1C2CCC1(*)CC2]

In some embodiments, n is 0. In some embodiments, n is 1.

In some embodiments, the reagent is chosen from a citric acid ester and a pinacol ester. In some embodiments, the reagent is citric acid.

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to ring A of Formula (I) and Formula (i) may be combined with every description, variation, embodiment, or aspect of X or Y or n, the same as if each and every combination were specifically and individually listed.

In some embodiments, provided is a compound chosen from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound | Structure | Name |
|---|---|---|
| A | 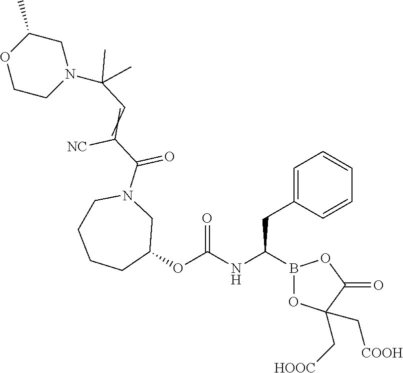 [SMILES: C[C@@H]1CN(C(C)(C)C=C(C#N)C(=O)N2CCCC[C@@H](OC(=O)N[C@@H](Cc3ccccc3)B3OC(=O)C(CC(=O)O)(CC(=O)O)O3)C2)CCO1] | 2,2′-(2-((R)-1-(((((R)-1-(2-cyano-4- methyl-4-((R)-2-methylmorpholino)pent- 2-enoyl)azepan-3- yl)oxy)carbonyl)amino)-2-phenylethyl)-5- oxo-1,3,2-dioxaborolane-4,4-diyl)diacetic acid |
| A-1 | 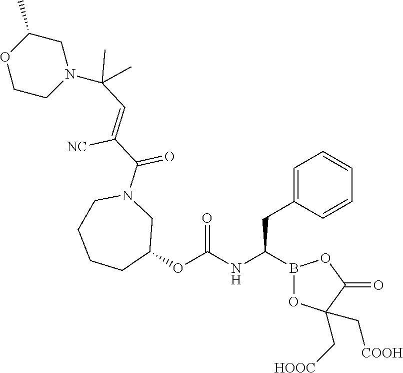 [SMILES: C[C@@H]1CN(C(C)(C)/C=C(\C#N)C(=O)N2CCCC[C@@H](OC(=O)N[C@@H](Cc3ccccc3)B3OC(=O)C(CC(=O)O)(CC(=O)O)O3)C2)CCO1] | 2,2′-(2-((R)-1-(((((R)-1-((E)-2-cyano-4- methyl-4-((R)-2-methylmorpholino)pent- 2-enoyl)azepan-3- yl)oxy)carbonyl)amino)-2-phenylethyl)-5- oxo-1,3,2-dioxaborolane-4,4-diyl)diacetic acid |
| B | 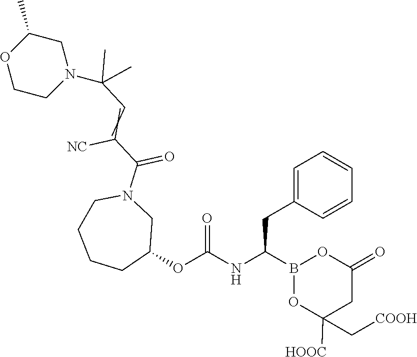 [SMILES: C[C@@H]1CN(C(C)(C)C=C(C#N)C(=O)N2CCCC[C@@H](OC(=O)N[C@@H](Cc3ccccc3)B3OC(=O)CC(CC(=O)O)(C(=O)O)O3)C2)CCO1] | 4-(carboxymethyl)-2-((R)-1-(((((R)-1-(2- cyano-4-methyl-4-((R)-2- methylmorpholino)pent-2-enoyl)azepan- 3-yl)oxy)carbonyl)amino)-2-phenylethyl)- 6-oxo-1,3,2-dioxaborinane-4-carboxylic acid |
| B-1 | 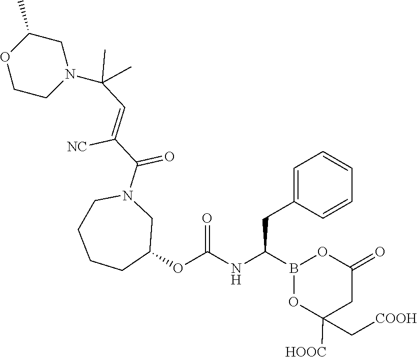 [SMILES: C[C@@H]1CN(C(C)(C)/C=C(\C#N)C(=O)N2CCCC[C@@H](OC(=O)N[C@@H](Cc3ccccc3)B3OC(=O)CC(CC(=O)O)(C(=O)O)O3)C2)CCO1] | 4-(carboxymethyl)-2-((R)-1-(((((R)-1- ((E)-2-cyano-4-methyl-4-((R)-2- methylmorpholino)pent-2-enoyl)azepan- 3-yl)oxy)carbonyl)amino)-2-phenylethyl)- 6-oxo-1,3,2-dioxaborinane-4-carboxylic acid |
| C | 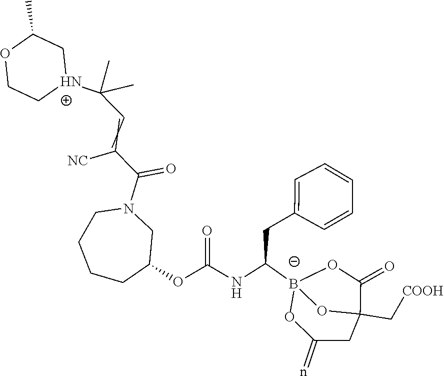 | 5-(carboxymethyl)-1-((1R)-1-(((((3R)-1- (2-cyano-4-methyl-4-((2R)-2- methylmorpholino-4-ium)pent-2- enoyl)azepan-3-yl)oxy)carbonyl)amino)- 2-phenylethyl)-3,6-dioxo-2,7,8-trioxa-1- borabicyclo[3.2.1]octan-1-uide |
| C-1 | 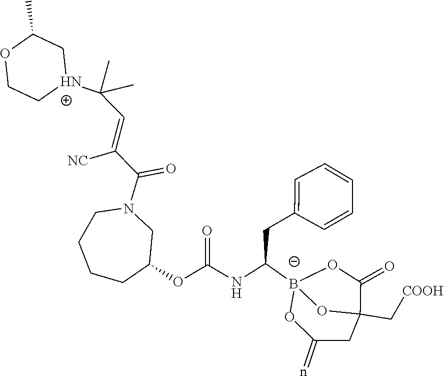 | 5-(carboxymethyl)-1-((1R)-1-(((((3R)-1- ((E)-2-cyano-4-methyl-4-((2R)-2- methylmorpholino-4-ium)pent-2- enoyl)azepan-3-yl)oxy)carbonyl)amino)- 2-phenylethyl)-3,6-dioxo-2,7,8-trioxa-1- borabicyclo[3.2.1]octan-1-uide |
| D | 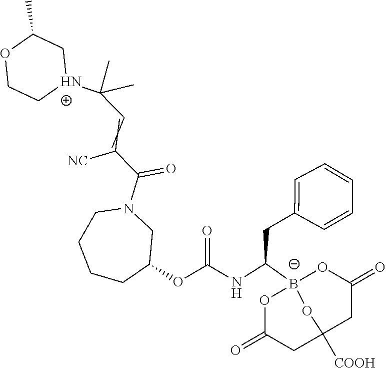 [SMILES: C[C@@H]1C[NH+](C(C)(C)C=C(C#N)C(=O)N2CCCC[C@@H](OC(=O)N[C@@H](Cc3ccccc3)[B-]34OC(=O)CC(C(=O)O)(CC(=O)O3)O4)C2)CCO1] | 5-carboxy-1-((1R)-1-(((((3R)-1-(2-cyano- 4-methyl-4-((2R)-2-methylmorpholino-4- ium)pent-2-enoyl)azepan-3- yl)oxy)carbonyl)amino)-2-phenylethyl)- 3,7-dioxo-2,8,9-trioxa-1- borabicyclo[3.3.1]nonan-1-uide |
| D-1 | 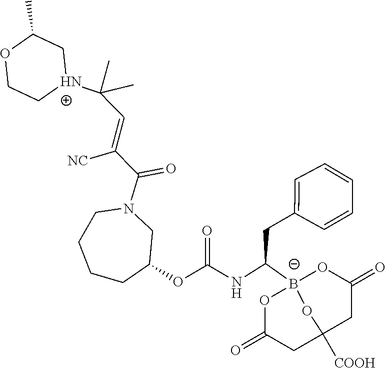 [SMILES: C[C@@H]1C[NH+](C(C)(C)/C=C(\C#N)C(=O)N2CCCC[C@@H](OC(=O)N[C@@H](Cc3ccccc3)[B-]34OC(=O)CC(C(=O)O)(CC(=O)O3)O4)C2)CCO1] | 5-carboxy-1-((1R)-1-(((((3R)-1-((E)-2- cyano-4-methyl-4-((2R)-2- methylmorpholino-4-ium)pent-2- enoyl)azepan-3-yl)oxy)carbonyl)amino)- 2-phenylethyl)-3,7-dioxo-2,8,9-trioxa-1- borabicyclo[3.3.1]nonan-1-uide |
| E | 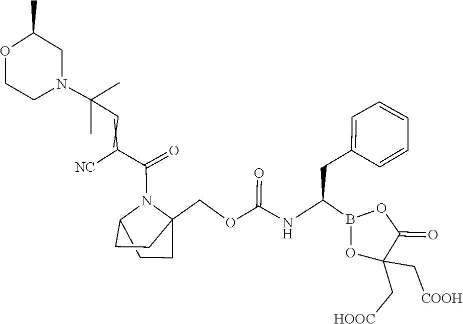 [SMILES: C[C@H]1CN(C(C)(C)C=C(C#N)C(=O)N2C3CCC2(COC(=O)N[C@@H](Cc2ccccc2)B2OC(=O)C(CC(=O)O)(CC(=O)O)O2)CC3)CCO1] | 2,2′-(2-((R)-1-((((7-(2-cyano-4-methyl-4- ((S)-2-methylmorpholino)pent-2-enoyl)-7- azabicyclo[2.2.1]heptan-1- yl)methoxy)carbonyl)amino)-2- phenylethyl)-5-oxo-1,3,2-dioxaborolane- 4,4-diyl)diacetic acid |
| F | 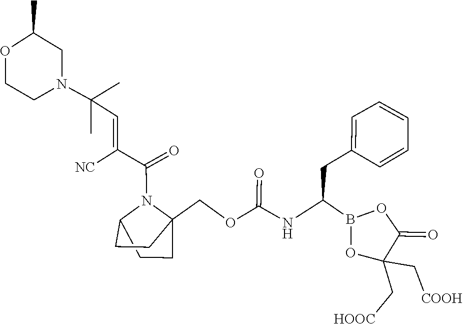 [SMILES: C[C@H]1CN(C(C)(C)/C=C(\C#N)C(=O)N2C3CCC2(COC(=O)N[C@@H](Cc2ccccc2)B2OC(=O)C(CC(=O)O)(CC(=O)O)O2)CC3)CCO1] | 4-(carboxymethyl)-2-((R)-1-((((7-(2- cyano-4-methyl-4-((S)-2- methylmorpholino)pent-2-enoyl)-7- azabicyclo[2.2.1]heptan-1- yl)methoxy)carbonyl)amino)-2- phenylethyl)-6-oxo-1,3,2-dioxaborinane- 4-carboxylic acid |
| G | 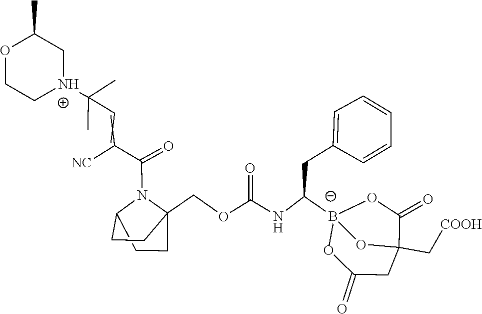 | 5-(carboxymethyl)-1-((1R)-1-((((7-(2- cyano-4-methyl-4-((2S)-2- methylmorpholino-4-ium)pent-2-enoyl)-7- azabicyclo[2.2.1]heptan-1- yl)methoxy)carbonyl)amino)-2- phenylethyl)-3,6-dioxo-2,7,8-trioxa-1- borabicyclo[3.2.1]octan-1-uide |
| H | 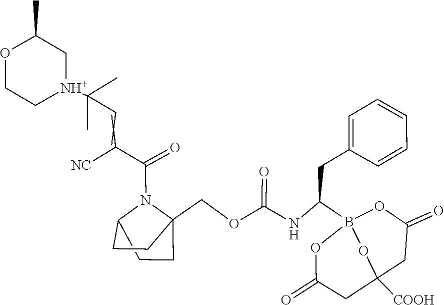 | 5-carboxy-1-((1R)-1-((((7-(2-cyano-4- methyl-4-((2S)-2-methylmorpholino-4- ium)pent-2-enoyl)-7- azabicyclo[2.2.1]heptan-1- yl)methoxy)carbonyl)amino)-2- phenylethyl)-3,7-dioxo-2,8,9-trioxa-1- borabicyclo[3.3.1]nonan-1-uide |
| I | 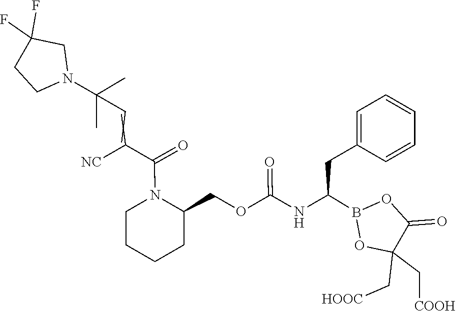 [SMILES: CC(C)(C=C(C#N)C(=O)N1CCCC[C@@H]1COC(=O)N[C@@H](Cc1ccccc1)B1OC(=O)C(CC(=O)O)(CC(=O)O)O1)N1CCC(F)(F)C1] | 2,2′-(2-((R)-1-(((((R)-1-(2-cyano-4-(3,3- difluoropyrrolidin-1-yl)-4-methylpent-2- enoyl)piperidin-2- yl)methoxy)carbonyl)amino)-2- phenylethyl)-5-oxo-1,3,2-dioxaborolane- 4,4-diyl)diacetic acid |
| J | 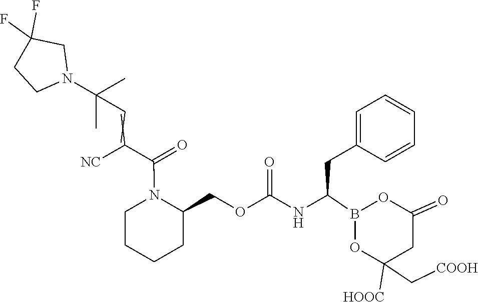 [SMILES: CC(C)(C=C(C#N)C(=O)N1CCCC[C@@H]1COC(=O)N[C@@H](Cc1ccccc1)B1OC(=O)CC(CC(=O)O)(C(=O)O)O1)N1CCC(F)(F)C1] | 4-(carboxymethyl)-2-((R)-1-(((((R)-1-(2- cyano-4-(3,3-difluoropyrrolidin-1-yl)-4- methylpent-2-enoyl)piperidin-2- yl)methoxy)carbonyl)amino)-2- phenylethyl)-6-oxo-1,3,2-dioxaborinane- 4-carboxylic acid |
| K | 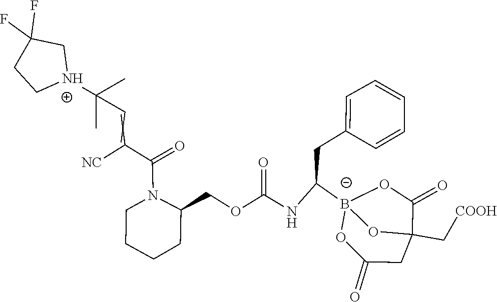 [SMILES: CC(C)(C=C(C#N)C(=O)N1CCCC[C@@H]1COC(=O)N[C@@H](Cc1ccccc1)[B-]12OC(=O)CC(CC(=O)O)(O1)C(=O)O2)[NH+]1CCC(F)(F)C1] | 5-(carboxymethyl)-1-((1R)-1-(((((2R)-1- (2-cyano-4-(3,3-difluoropyrrolidin-1-ium- 1-yl)-4-methylpent-2-enoyl)piperidin-2- yl)methoxy)carbonyl)amino)-2- phenylethyl)-3,6-dioxo-2,7,8-trioxa-1- borabicyclo[3.2.1]octan-1-uide |
| L | 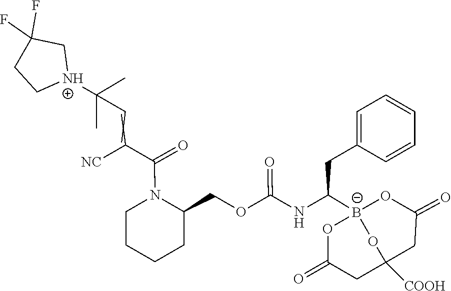 [SMILES: CC(C)(C=C(C#N)C(=O)N1CCCC[C@@H]1COC(=O)N[C@@H](Cc1ccccc1)[B-]12OC(=O)CC(C(=O)O)(CC(=O)O1)O2)[NH+]1CCC(F)(F)C1] | 5-carboxy-1-((1R)-1-(((((2R)-1-(2-cyano- 4-(3,3-difluoropyrrolidin-1-ium-1-yl)-4- methylpent-2-enoyl)piperidin-2- yl)methoxy)carbonyl)amino)-2- phenylethyl)-3,7-dioxo-2,8,9-trioxa-1- borabicyclo[3.3.1]nonan-1-uide |
| M | 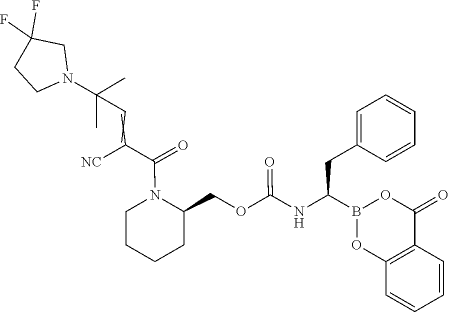 [SMILES: CC(C)(C=C(C#N)C(=O)N1CCCC[C@@H]1COC(=O)N[C@@H](Cc1ccccc1)B1OC(=O)c2ccccc2O1)N1CCC(F)(F)C1] | ((R)-1-(2-cyano-4-(3,3-difluoropyrrolidin- 1-yl)-4-methylpent-2-enoyl)piperidin-2- yl)methyl ((R)-1-(4-oxo-4H- benzo[d][1,3,2]dioxaborinin-2-yl)-2- phenylethyl)carbamate |
| N | 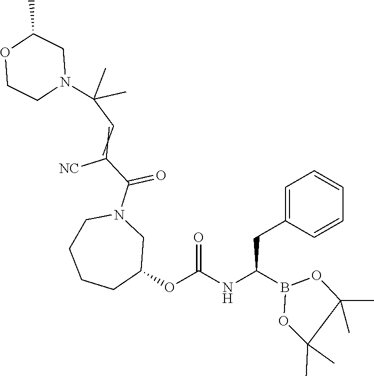 [SMILES: C[C@@H]1CN(C(C)(C)C=C(C#N)C(=O)N2CCCC[C@@H](OC(=O)N[C@@H](Cc3ccccc3)B3OC(C)(C)C(C)(C)O3)C2)CCO1] | (R)-1-(2-cyano-4-methyl-4-((R)-2- methylmorpholino)pent-2-enoyl)azepan- 3-yl ((R)-2-phenyl-1-(4,4,5,5-tetramethyl- 1,3,2-dioxaborolan-2-yl)ethyl)carbamate |

In some embodiments, the compound is

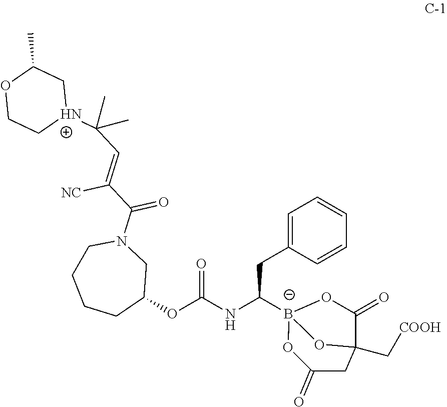 [SMILES: C[C@@H]1C[NH+](C(C)(C)/C=C(\C#N)C(=O)N2CCCC[C@@H](OC(=O)N[C@@H](Cc3ccccc3)[B-]34OC(=O)CC(CC(=O)O)(O3)C(=O)O4)C2)CCO1]

In one aspect, provided herein is a boronic acid ester of ((R)-1-(((((R)-1-(2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester is chosen from a citric acid ester, a pinacol ester, a malic acid ester, a salicylic acid ester, a lactic acid ester, and a tartaric acid ester. In some embodiments, the ester is chosen from a pinacol ester and a citric acid ester. In some embodiments, the ester is a citric acid ester.

In one aspect, provided herein is a boronic acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester is chosen from a citric acid ester, a pinacol ester, a malic acid ester, a salicylic acid ester, a lactic acid ester, and a tartaric acid ester. In some embodiments, the ester is chosen from a pinacol ester and a citric acid ester. In some embodiments, the ester is a citric acid ester.

In one aspect, provided herein is a boronic ester of [(IR)-1-[([[7-(2-cyano-2-[2-methyl-2-[(2S)-2-methylmorpholin-4-yl]propylidene]acetyl)-7-azabicyclo[2.2.1]heptan-1-yl]methoxy]carbonyl)amino]-2-phenylethyl]boronic acid or a pharmaceutically acceptable salt thereof, wherein the ester is chosen from a citric acid ester, a pinacol ester, a malic acid ester, a salicylic acid ester, a lactic acid ester, and a tartaric acid ester. In some embodiments, the ester is a citric acid ester.

In one aspect, provided herein is a boronic ester of ((R)-1-(((((R)-1-(2-cyano-4-(3,3-difluoropyrrolidin-1-yl)-4-methylpent-2-enoyl)piperidin-2-yl)methoxy)carbonyl)amino)-2-phenylethyl)boronic acid or a pharmaceutically acceptable salt thereof, wherein the ester is chosen from a citric acid ester, a pinacol ester, a malic acid ester, a salicylic acid ester, a lactic acid ester, and a tartaric acid ester. In some embodiments, the ester is a citric acid ester.

In one aspect, provided herein is a boronic acid ester of ((R)-1-(((((R)-1-(2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester converts to its corresponding acid upon exposure to physiological conditions. In some embodiments, the ester is chosen from a pinacol ester and a citric acid ester. In some embodiments, the ester is a citric acid ester.

In one aspect, provided herein is a boronic ester of ((R)-1-(((((R)-1-(2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester is stable at 40° C. and 75% relative humidity for at least three weeks. In some embodiments, the ester is chosen from a pinacol ester and a citric acid ester. In some embodiments, the ester is a citric acid ester.

In one aspect, provided herein is a boronic ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester a citric acid ester and is at least about 95% pure. In some embodiments, the ester is at least about 96% pure. In some embodiments, the ester is at least about 97% pure. In some embodiments, the ester is at least about 98% pure. In some embodiments, the ester is at least about 99% pure.

In one aspect, provided herein are crystalline forms of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid. In some embodiments, the crystalline form is chosen from Form I, Form II, Form III, Form IV and Form V.

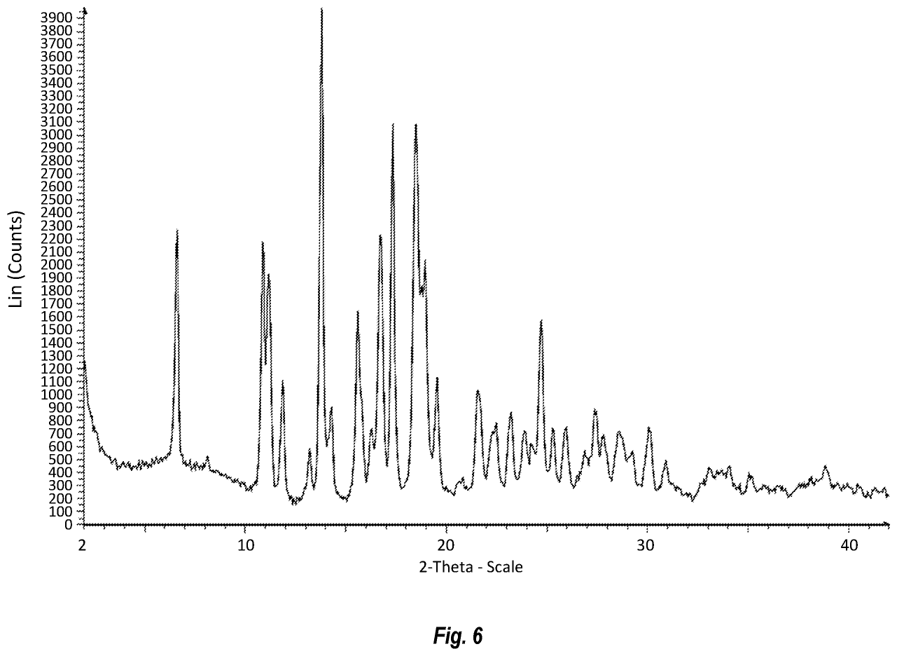

In some embodiments, the crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid is Form I. In some embodiments, Form I is characterized by an X-ray powder diffractogram comprising at least three peaks chosen from peaks at about 6.6±0.2, 11.2±0.2, 13.2±0.2, 13.8±0.2, 14.3±0.2, 15.6±0.2, 16.8±0.2, 17.5±0.2, 18.5±0.2, and 19.0±0.2 2-θ. In some embodiments, Form I is characterized by an X-ray powder diffractogram substantially similar to that in FIG. 6.

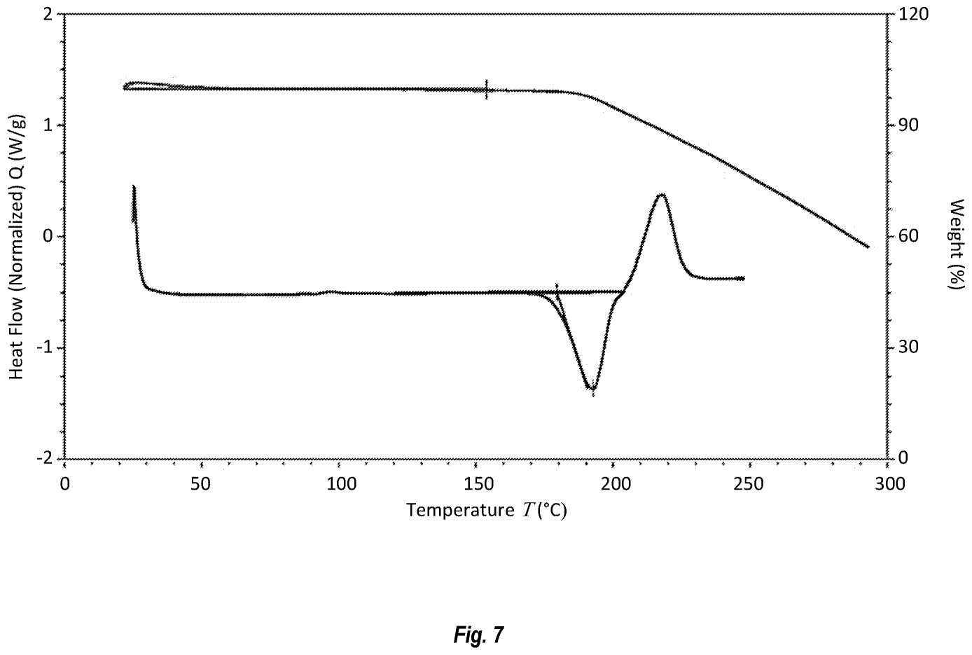

In some embodiments, Form I is characterized by a DSC thermogram having a peak endotherm at about 192.7° C. In some embodiments, Form I is characterized by a DSC thermogram showing onset of melting at about 179.5° C. In some embodiments, Form I is characterized by a DSC thermogram substantially similar to that in FIG. 7.

In some embodiments, Form I is characterized by a mass loss of less than about 0.5 wt. % between about 25° C. and about 150° C. by thermogravimetric analysis. In some embodiments, Form I is characterized by a TGA thermogram substantially similar to that in FIG. 7.

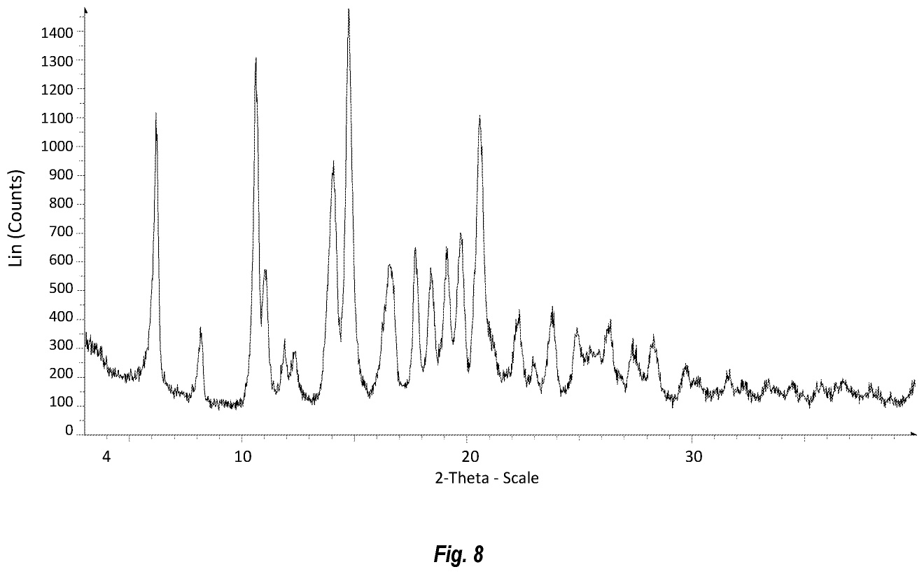

In some embodiments, the crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid is Form II. In some embodiments, Form II is characterized by an X-ray powder diffractogram comprising at least three peaks chosen from peaks at about 6.1±0.2, 8.1±0.2, 10.6±0.2, 11.0±0.2, 11.9±0.2, 14.0±0.2, 14.7±0.2, 16.6±0.2, 43.7±0.2, and 18.4±0.2 2-θ. In some embodiments, Form II is characterized by an X-ray powder diffractogram substantially similar to that in FIG. 8.

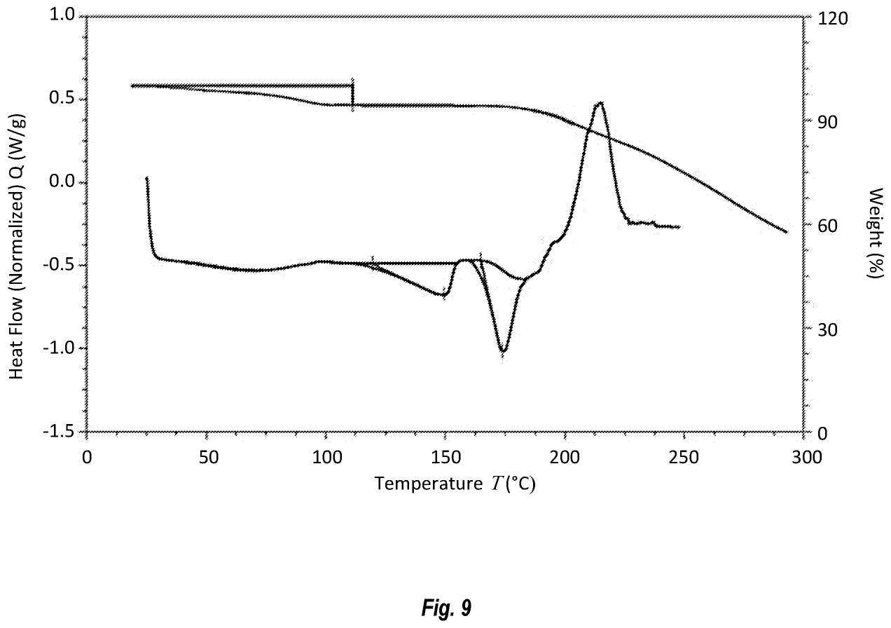

In some embodiments, Form II is characterized by a DSC thermogram having a peak endotherm at about 173.7° C. In some embodiments, Form II is characterized by a DSC thermogram showing onset of melting at about 164.9° C. In some embodiments, Form II is characterized by a DSC thermogram substantially similar to that in FIG. 9.

In some embodiments, Form II is characterized by a mass loss of about 5.6 wt. % between about 25° C. and about 110° C. by thermogravimetric analysis. In some embodiments, Form II is characterized by a TGA thermogram substantially similar to that in FIG. 9.

In some embodiments, Form I is prepared by contacting crystalline Form II with an alcohol or an alcohol mixture. In some embodiments, Form I is prepared by contacting crystalline Form III with an alcohol or an alcohol mixture. In some embodiments, the alcohol is EtOH. In some embodiments, the alcohol mixture comprises EtOH. In some embodiments, the alcohol mixture comprises EtOH and IPA.

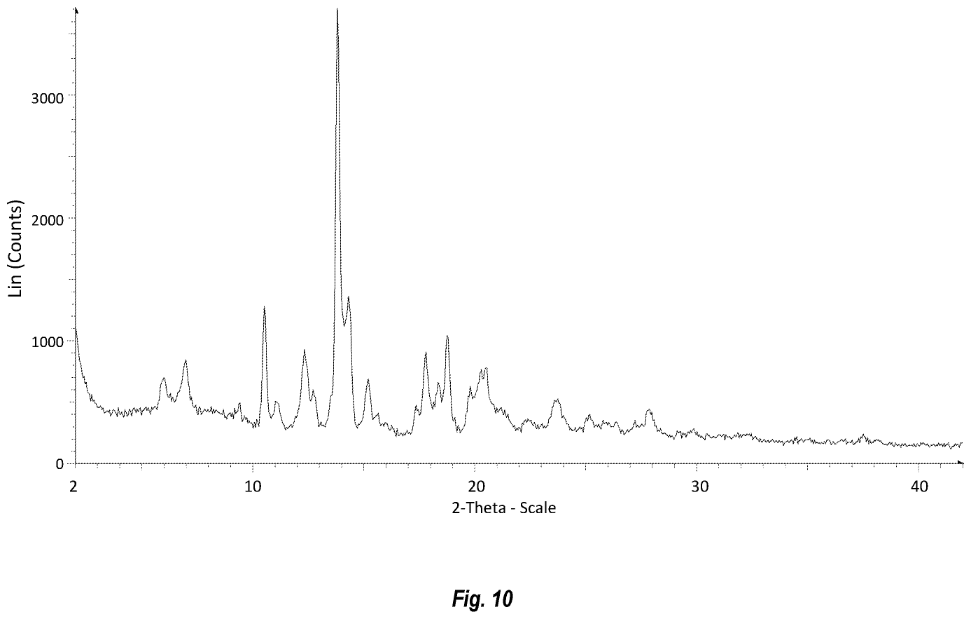

In some embodiments, the crystalline form of a citric acid ester of ((R)-1-(((((R)-1-((E)-2-cyano-4-methyl-4-((R)-2-methylmorpholino)pent-2-enoyl)azepan-3-yl)oxy) carbonyl)amino)-2-phenylethyl)boronic acid is Form III. In some embodiments, Form III is characterized by an X-ray powder diffractogram comprising at least three peaks chosen from peaks at about 5.9±0.2, 6.9±0.2, 9.4±0.2, 10.5±0.2, 11.1±0.2, 12.3±0.2, 12.7±0.2, 13.8±0.2, 14.3±0.2, and 15.2±0.2 2-θ. In some embodiments, Form III is characterized by an X-ray powder diffractogram substantially similar to that in FIG. 10.

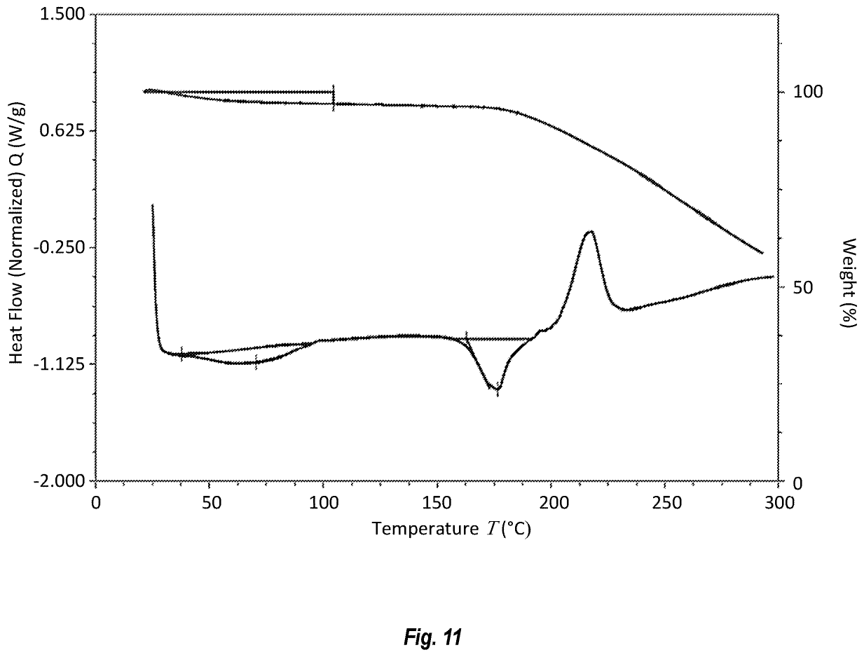

In some embodiments, Form III is characterized by a DSC thermogram having a peak endotherm at about 176.5° C. In some embodiments, Form III is characterized by a DSC thermogram having a broad endotherm from about 37.9° C. to about 100° C. In some embodiments, the broad endotherm has a peak at about 70.4° C. In some embodiments, Form III is characterized by a DSC thermogram substantially similar to that in FIG. 11.

In some embodiments, Form III is characterized by a mass loss of about 3.1 wt. % between about 25° C. and about 105° C. by thermogravimetric analysis. In some embodiments, Form III is characterized by a TGA thermogram substantially similar to that in FIG. 11.

In some embodiments, Form III is prepared by a process comprising contacting an alkyl ester, ether and/or toluene with Crystalline Form II. In some embodiments, the alkyl ester is chosen from ethyl acetate and isopropyl acetate. In some embodiments, the alkyl ester is isopropyl acetate. In some embodiments, the ether is methyl tert-butyl ether.

In some embodiments, the process further comprises isolating Form III from the alkyl ester, ether and/or toluene. In some embodiments, the alkyl ester is chosen from ethyl acetate and isopropyl acetate. In some embodiments, the alkyl ester is isopropyl acetate. In some embodiments, the ether is methyl tert-butyl ether.

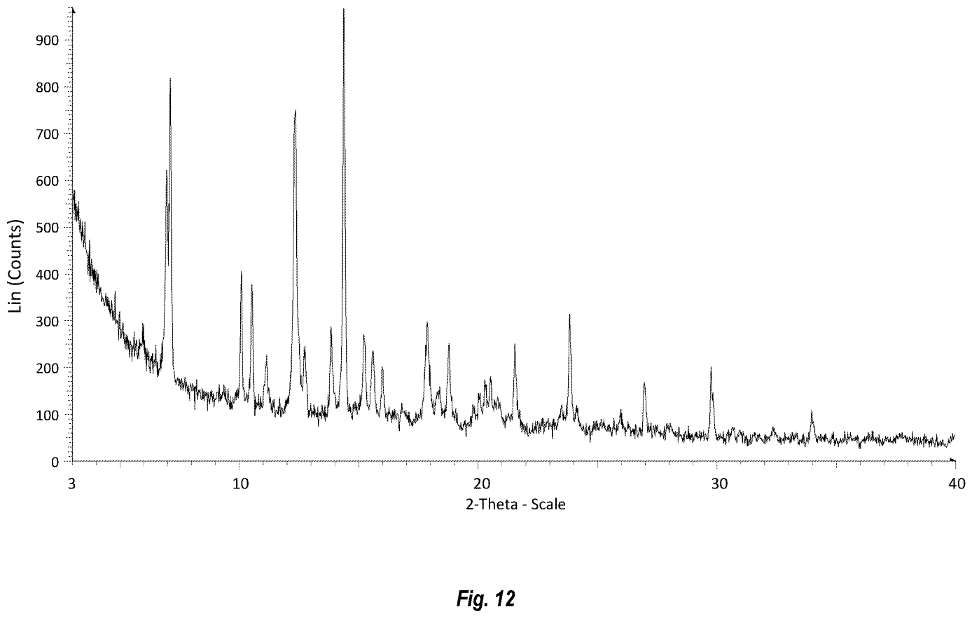

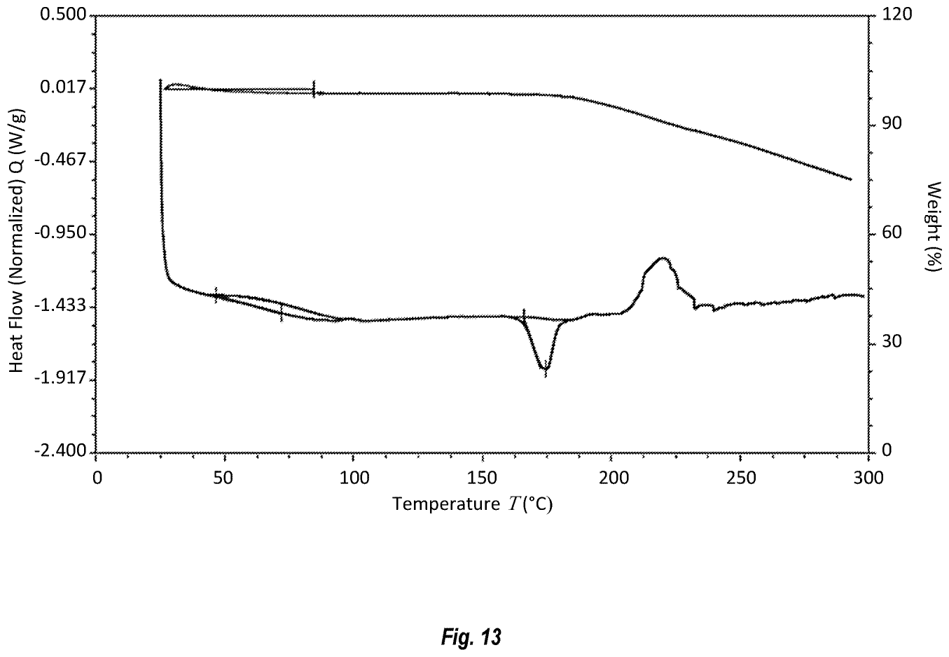

In some embodiments, the crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid is Form IV. In some embodiments, Form IV is characterized by an X-ray powder diffractogram substantially similar to that in FIG. 12. In some embodiments, Form IV is characterized by a DSC thermogram substantially similar to that in FIG. 13. In some embodiments, Form IV is characterized by a TGA thermogram substantially similar to that in FIG. 13.

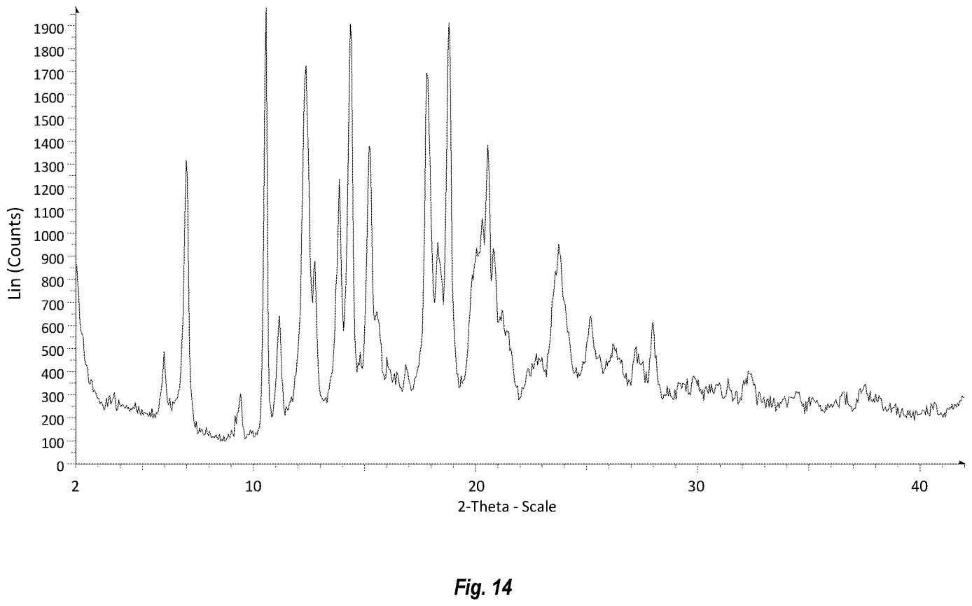

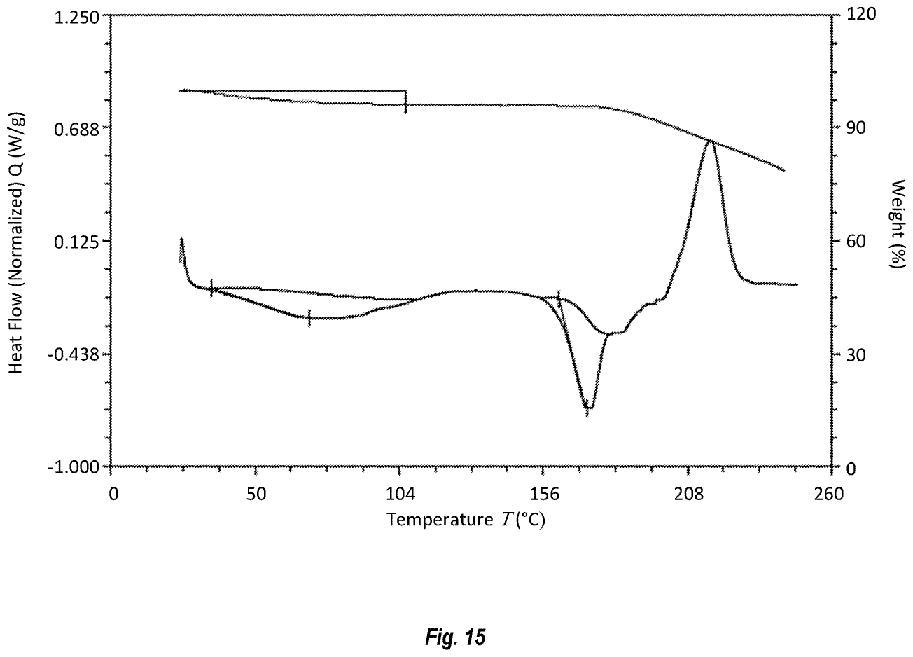

In some embodiments, the crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid is Form V. In some embodiments, Form V is characterized by an X-ray powder diffractogram substantially similar to that in FIG. 14. In some embodiments, Form V is characterized by a DSC thermogram substantially similar to that in FIG. 15. In some embodiments, Form V is characterized by a TGA thermogram substantially similar to that in FIG. 15.

### Pharmaceutical Compositions

In another aspect, provided herein are pharmaceutical compositions of the compounds described herein and a pharmaceutically acceptable salt thereof. Thus, the present disclosure includes pharmaceutical compositions comprising at least one compound of Formula (I) as described herein, or a pharmaceutically acceptable salt thereof, and a pharmaceutically acceptable excipient. The present disclosure further includes pharmaceutical compositions comprising at least one boronic acid as described herein, or a pharmaceutically acceptable salt thereof, and a pharmaceutically acceptable excipient. The present disclosure additionally includes pharmaceutical compositions comprising at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid as described herein, or a pharmaceutically acceptable salt thereof, and a pharmaceutically acceptable excipient.

Pharmaceutical compositions according to the disclosure may take a form suitable for oral, systemic (e.g., transdermal, intranasal or by suppository), parenteral (e.g., intramuscular, intravenous or subcutaneous) or topical (e.g., application to skin) administration. In some embodiments, the administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

Any compound described herein can be used in the preparation of a pharmaceutical composition by combining the compound as an active ingredient with a pharmaceutically acceptable excipient. Pharmaceutical formulations may be prepared by known pharmaceutical methods. Suitable formulations can be found in, for example, Remington: The Science and Practice of Pharmacy, Lippincott Williams & Wilkins, 21^{st} ed. (2005), which is incorporated herein by reference.

The pharmaceutical compositions may conveniently be presented in unit dosage form. Such dosage forms may be prepared by any methods well known in the art of pharmacy. The amount of active ingredient, which can be combined with an excipient(s) to produce a single dosage form, will vary depending upon the subject being treated and the particular mode of administration. The amount of active ingredient that can be combined with an excipient(s) to produce a single dosage form will generally be that amount of the compound which produces a therapeutic effect. Generally, this amount will range from about 1% to about 99% of active ingredient, for example from about 5% to about 70%, including from about 10% to about 30%.

In solid dosage forms of the disclosure for oral administration (capsules, tablets, pills, dragees, powders, granules and the like), the active ingredient may be mixed with one or more pharmaceutically-acceptable carriers, such as sodium citrate or dicalcium phosphate and/or any of the following: fillers or extenders, such as starches, lactose, sucrose, glucose, mannitol and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates and sodium carbonate; solution retarding agents, such as paraffin; wetting agents, such as, for example, cetyl alcohol, glycerol monostearate and non-ionic surfactants; absorbents, such as kaolin and bentonite clay; lubricants, such a talc, calcium stearate, magnesium stearate, solid polyethylene glycols, sodium lauryl sulfate and mixtures thereof, and coloring agents. In the case of capsules, tablets and pills, the pharmaceutical compositions may also comprise buffering agents.

### Methods of Treatment and Use as a Medicament

The compounds of described herein, or a pharmaceutically acceptable salt thereof, and pharmaceutical compositions comprising such compounds or a pharmaceutically acceptable salts thereof, may be used in methods of administration and treatment as provided herein. The compounds and pharmaceutical compositions may also be used in in vitro methods, such as in vitro methods of administering a compound or pharmaceutical composition to cells for screening purposes and/or for conducting quality control assays. Similarly, the compounds of described herein, or a pharmaceutically acceptable salt thereof, and pharmaceutical compositions comprising such compounds or a pharmaceutically acceptable salts thereof, may be used as medicaments for treatment as provided herein.

In one aspect, provided herein is a method of inhibiting Large Multifunctional Protease 2 (LMP2) in a subject comprising administering to said subject in need of said inhibition a therapeutically effective amount of at least one compounds described herein or a pharmaceutically acceptable salt thereof. Thus, the present disclosure includes a method of inhibiting Large Multifunctional Protease 2 (LMP2) in a subject comprising administering to said subject in need of said inhibition a therapeutically effective amount of at least one compound of Formula (I) as described herein, or a pharmaceutically acceptable salt thereof.

The present disclosure further includes a method of inhibiting Large Multifunctional Protease 2 (LMP2) in a subject comprising administering to said subject in need of said inhibition a therapeutically effective amount of at least one boronic acid as described herein, or a pharmaceutically acceptable salt thereof.

The present disclosure additionally includes a method of inhibiting Large Multifunctional Protease 2 (LMP2) in a subject comprising administering to said subject in need of said inhibition a therapeutically effective amount of at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid as described herein, or a pharmaceutically acceptable salt thereof.

In one aspect, provided herein is a method of inhibiting Large Multifunctional Protease 7 (LMP7) in a subject comprising administering to said subject in need of said inhibition a therapeutically effective amount of at least one compounds described herein or a pharmaceutically acceptable salt thereof. Thus, the present disclosure includes a method of inhibiting Large Multifunctional Protease 7 (LMP7) in a subject comprising administering to said subject in need of said inhibition a therapeutically effective amount of at least one compound of Formula (I) as described herein, or a pharmaceutically acceptable salt thereof.

The present disclosure further includes a method of inhibiting Large Multifunctional Protease 7 (LMP7) in a subject comprising administering to said subject in need of said inhibition a therapeutically effective amount of at least one boronic acid as described herein, or a pharmaceutically acceptable salt thereof.

The present disclosure additionally includes a method of inhibiting Large Multifunctional Protease 7 (LMP7) in a subject comprising administering to said subject in need of said inhibition a therapeutically effective amount of at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid as described herein, or a pharmaceutically acceptable salt thereof.

Inhibition of LMP2 and/or LMP7 can be assessed and demonstrated by a wide variety of ways known in the art. Kits and commercially available assays can be utilized for determining whether and to what degree LMP2 and/or LMP7 has been inhibited.

In another aspect, provided herein is a method for treating a disease associated with LMP2 and/or LMP7 in a subject in need thereof, comprising administering to the subject an effective amount of at least one of the compounds described herein or a pharmaceutically acceptable salt thereof. Non-limiting examples of a disease associated with LMP2 and/or LMP7 include rheumatoid arthritis and psoriatic arthritis, scleroderma, ankylosing spondylitis, Duchene muscular dystrophy (DMD), Becker muscular dystrophy (BMD), idiopathic inflammatory myopathies (IIMs), polymyositis, sporadic inclusion body myositis, dermatomyositis, immune-mediated necrotizing myopathies (IMNM), psoriasis, multiple sclerosis, inflammatory bowel disease, Behçet's disease, ulcerative colitis, Crohn's disease, Sjogren's Syndrome, bronchitis, conjunctivitis, pancreatitis, cholecystitis, bronchiectasis, aortic valve stenosis, restenosis, fibrosis, infection, ischemia, cardiovascular disease, hepatitis, cirrhosis, steatohepatitis, liver inflammation, Alzheimer's Disease (AD), amyotrophic lateral sclerosis (ALS), Huntington's disease, body myositis, myofibrilar myopathy, Graft-versus-Host Disease (GVHD), and multiple myeloma.

In another aspect, provided herein is a method for treating a disease chosen from an autoimmune disorder, an inflammatory disorder, and a hematological disorder in a patient in need of such treatment, comprising administering to the patient a therapeutically effective amount of at least one compound described herein, or a pharmaceutically acceptable salt thereof. Thus, the present disclosure includes a method for treating a disease chosen from an autoimmune disorder, an inflammatory disorder, and a hematological disorder in a patient in need of such treatment, comprising administering to the patient a therapeutically effective amount of at least one compound of Formula (I) as described herein, or a pharmaceutically acceptable salt thereof.

The present disclosure further includes a method for treating a disease chosen from an autoimmune disorder, an inflammatory disorder, and a hematological disorder in a patient in need of such treatment, comprising administering to the patient a therapeutically effective amount of at least one boronic acid as described herein, or a pharmaceutically acceptable salt thereof.

The present disclosure additionally includes a method for treating a disease chosen from an autoimmune disorder, an inflammatory disorder, and a hematological disorder in a patient in need of such treatment, comprising administering to the patient a therapeutically effective amount of at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid as described herein, or a pharmaceutically acceptable salt thereof.

In some embodiments, the disease is chosen from lupus, arthritis including rheumatoid arthritis and psoriatic arthritis, scleroderma, ankylosing spondylitis, Duchene muscular dystrophy (DMD), Becker muscular dystrophy (BMD), idiopathic inflammatory myopathies (IIMs), polymyositis, sporadic inclusion body myositis, dermatomyositis, immune-mediated necrotizing myopathies (IMNM), psoriasis, multiple sclerosis, inflammatory bowel disease, Behçet's disease, ulcerative colitis, Crohn's disease, Sjogren's Syndrome, bronchitis, conjunctivitis, pancreatitis, cholecystitis, bronchiectasis, aortic valve stenosis, restenosis, fibrosis, infection, ischemia, cardiovascular disease, hepatitis, cirrhosis, steatohepatitis, liver inflammation, Alzheimer's Disease (AD), amyotrophic lateral sclerosis (ALS), Huntington's disease, body myositis, myofibrillar myopathy, Graft-versus-Host Disease (GVHD), and multiple myeloma.

In another aspect, provided herein is a use of a composition as a medicament for inhibiting Large Multifunctional Protease 2 (LMP2) in a subject, wherein the composition comprises a therapeutically effective amount of a compound described herein or a pharmaceutically acceptable salt thereof. Thus, the present disclosure includes a use of a composition as a medicament for inhibiting Large Multifunctional Protease 2 (LMP2) in a subject, wherein the composition comprises a therapeutically effective amount of at least one compound of Formula (I) as described herein, or a pharmaceutically acceptable salt thereof.

The present disclosure further includes a use of a composition as a medicament for inhibiting Large Multifunctional Protease 2 (LMP2) in a subject, wherein the composition comprises a therapeutically effective amount of at least one boronic ester as described herein, or a pharmaceutically acceptable salt thereof.

The present disclosure additionally includes a use of a composition as a medicament for inhibiting Large Multifunctional Protease 2 (LMP2) in a subject, wherein the composition comprises a therapeutically effective amount of at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid as described herein, or a pharmaceutically acceptable salt thereof.

In one aspect, provided herein is a use of a composition as a medicament for inhibiting Large Multifunctional Protease 7 (LMP7) in a subject, wherein the composition comprises a therapeutically effective amount of at least one of the compounds described herein or a pharmaceutically acceptable salt thereof. Thus, the present disclosure includes a use of a composition as a medicament for inhibiting Large Multifunctional Protease 7 (LMP7) in a subject, wherein the composition comprises a therapeutically effective amount of at least one compound of Formula (I) as described herein, or a pharmaceutically acceptable salt thereof.

The present disclosure further includes a use of a composition as a medicament for inhibiting Large Multifunctional Protease 7 (LMP7) in a subject, wherein the composition comprises a therapeutically effective amount of at least one boronic ester as described herein, or a pharmaceutically acceptable salt thereof.

The present disclosure additionally includes a use of a composition as a medicament for inhibiting Large Multifunctional Protease 7 (LMP7) in a subject, wherein the composition comprises a therapeutically effective amount of at least one crystalline form of a citric acid ester of ((R)-1-(((((R)-1-((E)-2-cyano-4-methyl-4-((R)-2-methylmorpholino)pent-2-enoyl)azepan-3 -yl)oxy)carbonyl)amino)-2-phenylethyl)boronic acid as described herein, or a pharmaceutically acceptable salt thereof.

In one aspect, provided herein is a use of a composition as a medicament for treating a disease in a subject in need therefore, wherein the composition comprises administering to the patient a therapeutically effective amount of at least one of the compounds described herein or a pharmaceutically acceptable salt thereof; and wherein the disease chosen from an autoimmune disorder, an inflammatory disorder, and a hematological disorder. Thus, the present disclosure includes a use of a composition as a medicament for treating a disease in a subject in need therefore, wherein the composition comprises administering to the patient a therapeutically effective amount of at least one compound of Formula (I) as described herein, or a pharmaceutically acceptable salt thereof; and wherein the disease chosen from an autoimmune disorder, an inflammatory disorder, and a hematological disorder.

The present disclosure further includes a use of a composition as a medicament for treating a disease in a subject, wherein the composition comprises a therapeutically effective amount of at least one boronic ester as described herein, or a pharmaceutically acceptable salt thereof; and wherein the disease chosen from an autoimmune disorder, an inflammatory disorder, and a hematological disorder.

The present disclosure additionally includes a use of a composition as a medicament for treating a disease in a subject, wherein the composition comprises a therapeutically effective amount of at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid as described herein, or a pharmaceutically acceptable salt thereof; and wherein the disease chosen from an autoimmune disorder, an inflammatory disorder, and a hematological disorder.

In some embodiments, the disease is chosen from lupus, arthritis including rheumatoid arthritis and psoriatic arthritis, scleroderma, ankylosing spondylitis, Duchene muscular dystrophy (DMD), Becker muscular dystrophy (BMD), idiopathic inflammatory myopathies (IIMs), polymyositis, sporadic inclusion body myositis, dermatomyositis, immune-mediated necrotizing myopathies (IMNM), psoriasis, multiple sclerosis, inflammatory bowel disease, Behçet's disease, ulcerative colitis, Crohn's disease, Sjogren's Syndrome, bronchitis, conjunctivitis, pancreatitis, cholecystitis, bronchiectasis, aortic valve stenosis, restenosis, fibrosis, infection, ischemia, cardiovascular disease, hepatitis, cirrhosis, steatohepatitis, liver inflammation, Alzheimer's Disease (AD), amyotrophic lateral sclerosis (ALS), Huntington's disease, body myositis, myofibrilar myopathy, Graft-versus-Host Disease (GVHD), and multiple myeloma.

In some embodiments, administering a compound described herein, or a pharmaceutically acceptable salt thereof, to a subject in need thereof diminishes the extent of a disease associated with LMP2 and/or LMP7 (for example, those listed above) in the subject. In some embodiments, administering a compound described herein, or a pharmaceutically acceptable salt thereof, to a subject in need thereof stabilizes a disease associated with LMP2 and/or LMP7. In some embodiments, administering a compound described herein, or a pharmaceutically acceptable salt thereof, to a subject in need thereof delays the occurrence or recurrence of a disease associated with LMP2 and/or LMP7. In some embodiments, administering a compound described herein, or a pharmaceutically acceptable salt thereof, to a subject in need thereof slows the progression of a disease associated with LMP2 and/or LMP7. In some embodiments, administering a compound described herein, or a pharmaceutically acceptable salt thereof, to a subject in need thereof delays the progression of a disease associated with LMP2 and/or LMP7. In some embodiments, administering a compound described herein, or a pharmaceutically acceptable salt thereof, to a subject in need thereof increases the quality of life of the subject having a disease associated with LMP2 and/or LMP7. In some embodiments, administering a compound described herein, or a pharmaceutically acceptable salt thereof, to a subject in need thereof prolongs survival of a subject having a disease associated with LMP2 and/or LMP7.

### Dosing and Method of Administration

These compounds may be administered to humans for therapy by any suitable route of administration, including orally, nasally, as by, for example, a spray, rectally, intravaginally, parenterally, intracistemally and topically, as by powders, ointments or drops, including buccally and sublingually. The unit administration forms appropriate include oral forms such as tablets, soft or hard gel capsules, powders, granules and oral solutions or suspensions, sublingual, buccal, intratracheal, intra-ocular and intranasal administration forms, forms for inhalative, topical, transdermal, subcutaneous, intra-muscular or intravenous administration, rectal administration forms and implants. For topical application it is possible to use the herein described compounds in creams, gels, ointments or lotions.

Regardless of the route of administration selected, the compounds described herein, or pharmaceutically acceptable salts thereof, are formulated into pharmaceutically acceptable dosage forms by conventional methods known to those of skill in the art.

Actual dosage levels of the active ingredients in the pharmaceutical compositions of this disclosure may be varied so as to obtain an amount of the active ingredient that is effective to achieve the desired therapeutic response for a particular patient, composition and mode of administration, without being toxic to the patient.

The selected dosage level will depend upon a variety of factors including the activity of the particular compound or salt of the present disclosure employed, the route of administration, the time of administration, the rate of excretion or metabolism of the particular compound or salt being employed, the duration of the treatment, other drugs, compounds and/or materials used in combination with the particular compound or salt employed, the age, sex, weight, condition, general health and prior medical history of the patient being treated and like factors well known in the medical arts. A daily, weekly or monthly dosage (or other time interval) can be used.

A physician having ordinary skill in the art can readily determine and prescribe the effective amount of the pharmaceutical composition required. For example, the physician could start doses of the compounds or salts of the disclosure employed in the pharmaceutical composition at levels lower than that required to achieve the desired therapeutic effect and then gradually increasing the dosage until the desired effect is achieved.

In general, a suitable daily dose of a compound of the disclosure will be that amount of the compound that is the lowest dose effective to produce a therapeutic effect (e.g., inhibit necrosis). Such an effective dose will generally depend upon the factors described above. Generally, doses of the compounds of this disclosure for a patient, when used for the indicated effects, will range from about 0.0001 to about 100 mg per kg of body weight per day. Preferably the daily dosage will range from 0.001 to 50 mg of compound per kg of body weight and even more preferably from 0.01 to 10 mg of compound per kg of body weight.

If desired, the effective daily dose of the active compound may be administered as two, three, four, five, six or more sub-doses administered separately at appropriate intervals throughout the day, optionally, in unit dosage forms.

When the compounds or salts of the present disclosure are administered as pharmaceuticals to humans, they can be given per se or as a pharmaceutical composition containing, for example, 0.1% to 99.5% (such as 0.5% to 90%) of active ingredient in combination with a pharmaceutically acceptable carrier.

The compounds and salts of the present application or the pharmaceutical compositions thereof may be administered once, twice, three, or four times daily, using any suitable mode described above. Also, administration or treatment with the compounds or salts may be continued for a number of days; for example, commonly treatment would continue for at least 7 days, 14 days, or 28 days, for one cycle of treatment. Treatment cycles are well known and are frequently alternated with resting periods of about 1 to 28 days, commonly about 7 days or about 14 days, between cycles. The treatment cycles, in certain embodiments, may also be continuous.

In certain embodiments, the methods and uses herein comprise administering to the subject an initial daily dose of about 1 to 800 mg of a compound or salt described herein and increasing the dose by increments until clinical efficacy is achieved. Increments of about 5, 10, 25, 50 or 100 mg can be used to increase the dose. The dosage can be increased daily, every other day, twice per week or once per week.

The preparations of the present disclosure may be given orally, parenterally, topically, or rectally. They are, of course, given in forms suitable for each administration route. For example, they are administered in tablets or capsule form; by injection, inhalation, eye lotion, ointment, suppository, infusion, inhalation, etc.; topical by lotion or ointment; and rectal by suppositories. In certain embodiments, the administration is oral.

The methods of the present disclosure may include a compound described herein, or a pharmaceutically acceptable salt thereof, used alone or in combination with one or more additional therapies (e.g., non-drug treatments or therapeutic agents). The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis.

The compounds described herein, or salts thereof, may be administered before, after, or concurrently with one or more of such additional therapies. When combined, dosages of the compounds described herein, or salts thereof, and dosages of the one or more additional therapies (e.g., non-drug treatment or therapeutic agent) provide a therapeutic effect (e.g., synergistic or additive therapeutic effect). A compound described herein, or a pharmaceutically acceptable salt thereof, and an additional therapy, such as an anti-cancer agent, may be administered together, such as in a unitary pharmaceutical composition, or separately and, when administered separately, this may occur simultaneously or sequentially. Such sequential administration may be close or remote in time.

### Examples

The examples and preparations provided below further illustrate and exemplify the compounds of the present disclosure. It is to be understood that the scope of the present disclosure is not limited in any way by the scope of the following examples.

The chemical reactions in the Examples described can be readily adapted to prepare a number of other compounds described herein, and alternative methods for preparing the compounds of this disclosure are deemed to be within the scope of this disclosure. For example, the synthesis of non-exemplified compounds according to the present disclosure can be performed by modifications apparent to those skilled in the art, for example by appropriately protecting interfering groups, by utilizing other suitable reagents known in the art other than those described, or by making routine modification of reaction conditions, reagents, and starting materials. Alternatively, other reactions described herein or known in the art will be recognized as having applicability for preparing other compounds of the present disclosure. The following abbreviations may be relevant for the application.

### Abbreviations

ACN and MeCN Acetonitrile
BOP—Cl Bis (2-oxo-3-oxazolidinyl)phosphinic chloride
i-BuB(OH)₂ Isobutylboronic acid
DCM Dichloromethane
DIPEA N,N-Diisopropylethylamine
DMAP 4-Dimethylaminopyridine
EtOAc and EA Ethyl acetate
EtOH Ethanol
HATU 1-[Bis(dimethylamino)methylene]-1H-1,2,3-triazolo[4,5-b]pyridinium 3-oxide hexafluorophosphate
IPA 2-Propanol
IPAC Isopropyl Acetate
MEK 2-Butanone
MeOH Methanol
MTBE Methyl t-Butyl Ether
SGF Simulated Gastric Fluid
SIF Simulated Intestinal Fluid
THF Tetrahydrofuran
TMBH (1α,2α,3α,5α)]-2,6,6-trimethylbicyclo[3.1.1]heptane-2,3-diol (i.e., pinanediol)
TMSCl Trimethylsilyl chloride
UPLC Ultra-Performance Liquid Chromatography

### Example 1: Preparation of Citric Acid Esters of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid

### Step 1: Synthesis of Starting Material 1 (SM1)

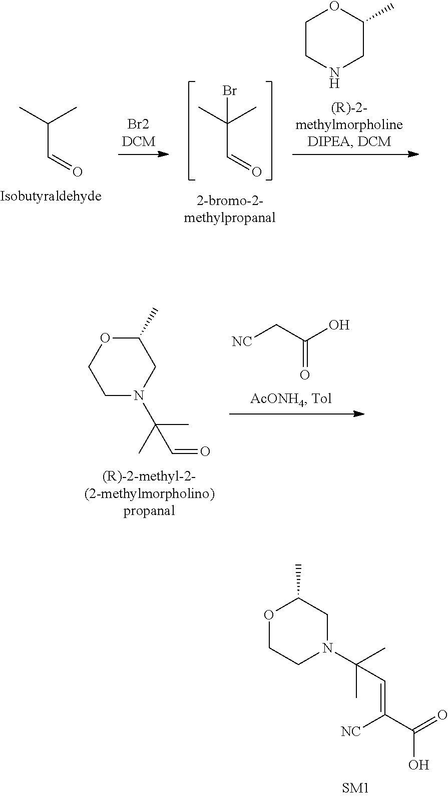

Isobutyraldehyde (733g) in DCM (3500 mL) was brominated with bromine (1686g) at 0 to 5° C. The reaction was quenched with aqueous Na₂SO₃ and washed with water and brine. The DCM solution was stored at 0 to 10° C. (R)-2-Methylmorpholine (700g) was dissolved in DCM (1400 mL), cooled to 5° C., and diispropylethylamine (1.97L) was added. The reaction was cooled to 0 to 5° C., followed by the addition of the DCM solution of 2-bromo-2-methylpropanal. The layers were separated, and the organic phase was washed with water and brine. The organic phase was concentrated under reduced pressure to dryness to give (R)-2-methyl-2-(2-methylmorpholino) propanal (802g) as a light-yellow oil. Yield: 90% yield and Purity: 99.0%. (R)-2-Methyl-2-(2-methylmorpholino) propanal (802g) and 2-cyanoacetic acid (439g) were dissolved in toluene (2.4L), followed by the addition of ammonium acetate (18g). The reaction was heated to 80±5° C. for 22 hours, then cooled to 20±5° C. and acetonitrile (1.6L) was added, forming a solid. The solid was collected by filtration and rinsed with acetonitrile. The solid was dried under vacuum at 40±5° C. to constant weight to afford SM1 (790g) as a white solid. Yield: 70% yield and Purity: 98.5%.

### Step 2: Synthesis of Intermediate 3

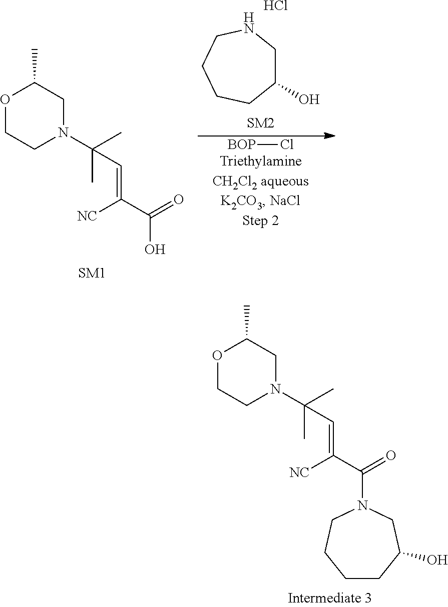

Starting material 1 (SM1) was combined with starting material 2 (SM2) (which can be prepared according to methods disclosed in the literature), BOP—Cl, and triethyl amine in CH₂Cl₂. The reaction was washed with aqueous K₂CO₃ and NaCl to yield Intermediate 3.

### Step 3: Synthesis of Intermediate 5

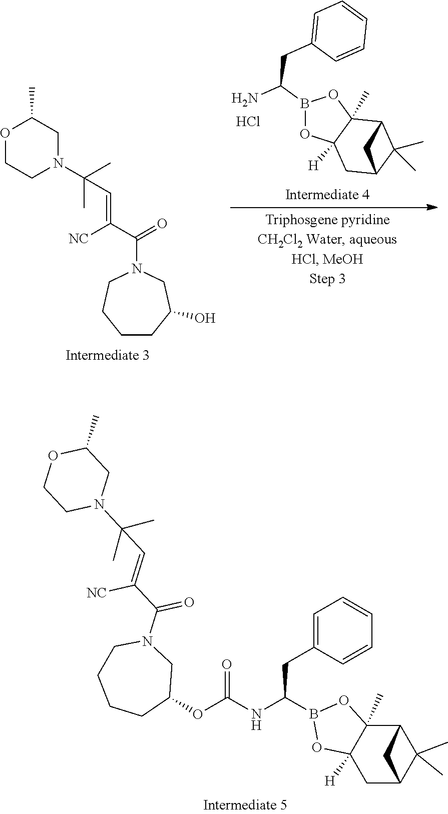 [SMILES: C[C@@H]1CN(C(C)(C)/C=C(\C#N)C(=O)N2CCCC[C@@H](O)C2)CCO1 ; Cl ; CC1(C)[C@@H]2C[C@H]3OB([C@@H](N)Cc4ccccc4)O[C@@]3(C)[C@H]1C2 ; C[C@@H]1CN(C(C)(C)/C=C(\C#N)C(=O)N2CCCC[C@@H](OC(=O)N[C@@H](Cc3ccccc3)B3O[C@@H]4C[C@@H]5C[C@@H](C5(C)C)[C@]4(C)O3)C2)CCO1]

Intermediate 3 was combined with Intermediate 4 (which was obtained from KingChem Life Science and can be prepared by known methods, for example, as in U.S. Pat. No. 11,225,493), triphosgene, and pyridine in CH₂Cl₂. The reaction mixture was then combined with water, aqueous HCl and MeOH to yield Intermediate 5.

### Step 4: Synthesis of Boronic Acid 1

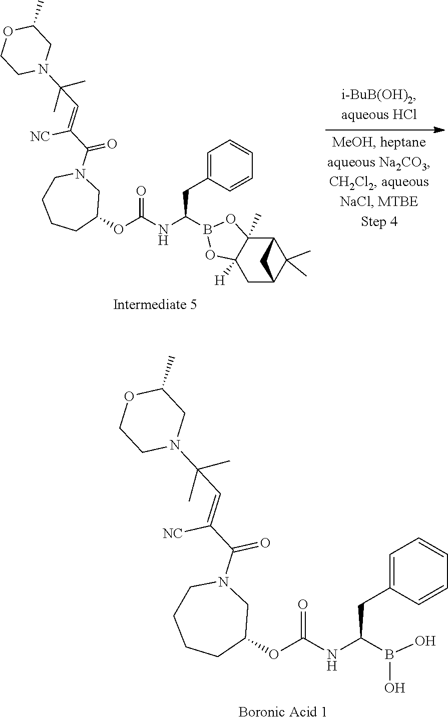 [SMILES: C[C@@H]1CN(C(C)(C)/C=C(\C#N)C(=O)N2CCCC[C@@H](OC(=O)N[C@@H](Cc3ccccc3)B3O[C@@H]4C[C@@H]5C[C@@H](C5(C)C)[C@]4(C)O3)C2)CCO1 ; C[C@@H]1CN(C(C)(C)/C=C(\C#N)C(=O)N2CCCC[C@@H](OC(=O)N[C@@H](Cc3ccccc3)B(O)O)C2)CCO1]

Intermediate 5 was combined with i-BuB(OH)₂ in aqueous HCl, MeOH, and heptane. The mixture was then mixed with aqueous NaCO₃, CH₂Cl₂, aqueous NaCl and MTBE to yield Boron Acid 1 (i.e., ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid).

### Step 5: Synthesis of Citric Acid Esters of Boronic Acid 1

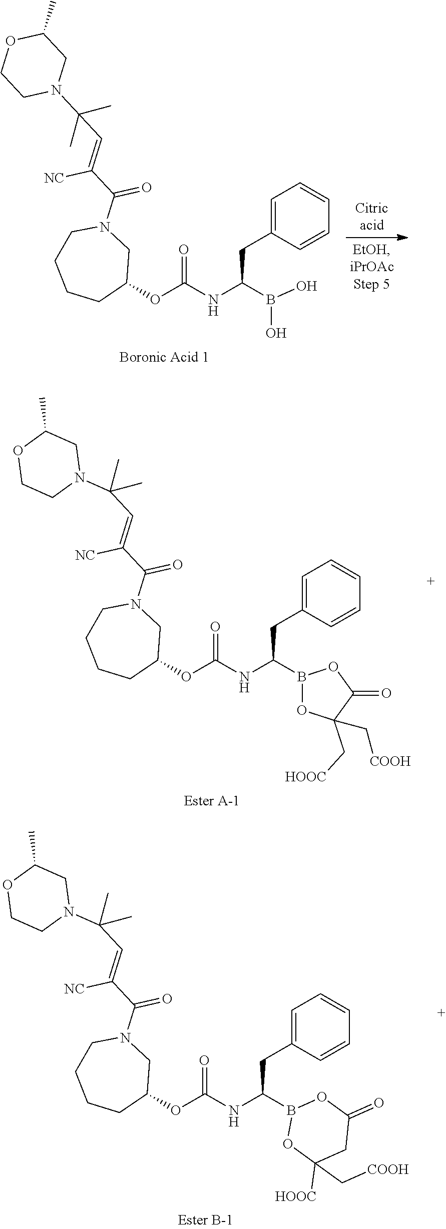 [SMILES: C[C@@H]1CN(C(C)(C)/C=C(\C#N)C(=O)N2CCCC[C@@H](OC(=O)N[C@@H](Cc3ccccc3)B(O)O)C2)CCO1 ; C[C@@H]1CN(C(C)(C)/C=C(\C#N)C(=O)N2CCCC[C@@H](OC(=O)N[C@@H](Cc3ccccc3)B3OC(=O)C(CC(=O)O)(CC(=O)O)O3)C2)CCO1 ; C[C@@H]1CN(C(C)(C)/C=C(\C#N)C(=O)N2CCCC[C@@H](OC(=O)N[C@@H](Cc3ccccc3)B3OC(=O)CC(CC(=O)O)(C(=O)O)O3)C2)CCO1]

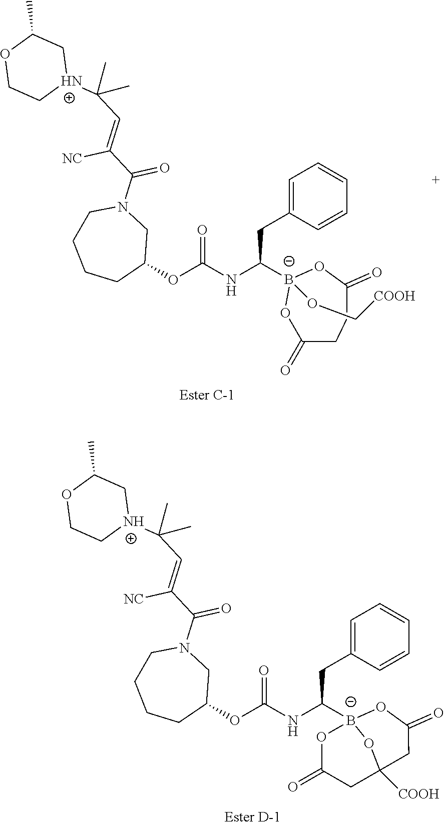 [SMILES: C[C@@H]1C[NH+](C(C)(C)/C=C(\C#N)C(=O)N2CCCC[C@@H](OC(=O)N[C@@H](Cc3ccccc3)[B-]3(OCC(=O)O)OC(=O)CCC(=O)O3)C2)CCO1 ; C[C@@H]1C[NH+](C(C)(C)/C=C(\C#N)C(=O)N2CCCC[C@@H](OC(=O)N[C@@H](Cc3ccccc3)[B-]34OC(=O)CC(C(=O)O)(CC(=O)O3)O4)C2)CCO1]

A solution of anhydrous citric acid (1.05 eq.) in ethanol (EtOH, 5 mL) was added to a solution of Boron Acid 1 (1.0 g) in isopropyl acetate (IPAC, 5 mL) at 15-20° C. and stirred for 5 min to a clear solution. The solution was then warmed to 30-35° C. and stirred until crystals formed. The slurry was stirred for about 2 h at 30-35° C. and then cooled slowly to 15-20° C. and stirred for 2 h. The resulting slurry was filtered and the solid was washed with ethanol/IPAC (1/1.2 mL). The solid was then dried at 30-35° C. under vacuum yielding a mixture of the citric acid esters shown above (Esters A-1 to D-1) as an off-white solid (1.0 g, 77% yield, 99% purity). MS [M+1]: 683 (SFC-MS). The 1H NMR results are shown in Table 1, below.

TABLE 1

| Chemical Shift (ppm) | Split | Hydrogen Number | Assignment |
|---|---|---|---|
| 11.96 | s | 1H | OH-39 |
| 9.82 | s | 1H | NH-1 |
| 7.18 | overlap | 4H | H-24, 25, 27, 28 |
| 7.09 | overlap | 1H | H-26 |
| 7.09 | overlap | 1H | H-4 |
| 5.70 | br s | 1H | NH-20* |
| 4.59 | br s | 1H | H-12 |
| 4.01 | br s | 1H | H-44a |
| 3.86 | overlap | 1H | H-45a |
| 3.76 | overlap | 1H | H-42 |
| 3.69 | overlap | 1H | H-44b |
| 3.69 | overlap | 1H | H-11a |
| 3.56 | overlap | 1H | H-11b |
| 3.56 | overlap | 1H | H-41a |
| 3.52 | overlap | 1H | H-45b |
| 3.49 | overlap | 2H | H-16 |
| 3.03 | overlap | 1H | H-21 |
| 2.87 | overlap | 1H | H-41b |
| 2.78 | overlap | 1H | H-22a |
| 2.62 | overlap | 4H | H-37, 38* |
| 2.46 | overlap | 1H | H-22b |
| 1.71 | overlap | 2H | H-15 |
| 1.71 | overlap | 6H | H-3, 5 |
| 1.62 | overlap | 1H | H-14a |
| 1.59 | overlap | 1H | H-13a |
| 1.52 | overlap | 1H | H-13b |
| 1.41 | overlap | 1H | H-14b |
| 1.16 | overlap | 3H | H-46 |

*The assignment of the signal could not be confirmed.

### Example 2: Preparation of a Pinacol ester of ((R)-1-(((((R)-1-(2-cyano-4-methyl-4-((R)-2-methylmorpholino)pent-2-enoyl)azepan-3-yl)oxy)carbonyl)amino)-2-phenylethyl) boronic acid

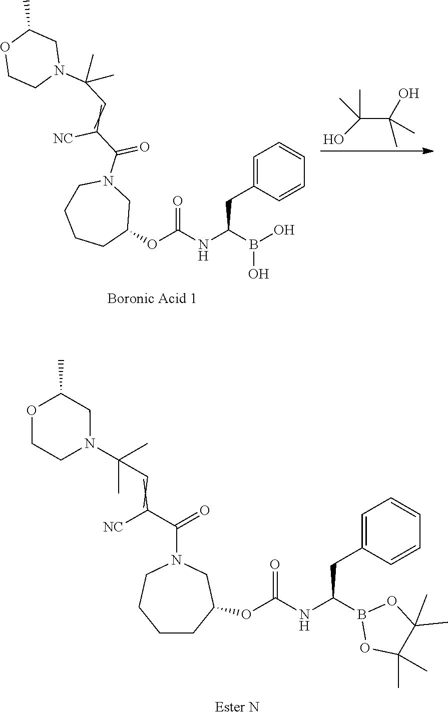

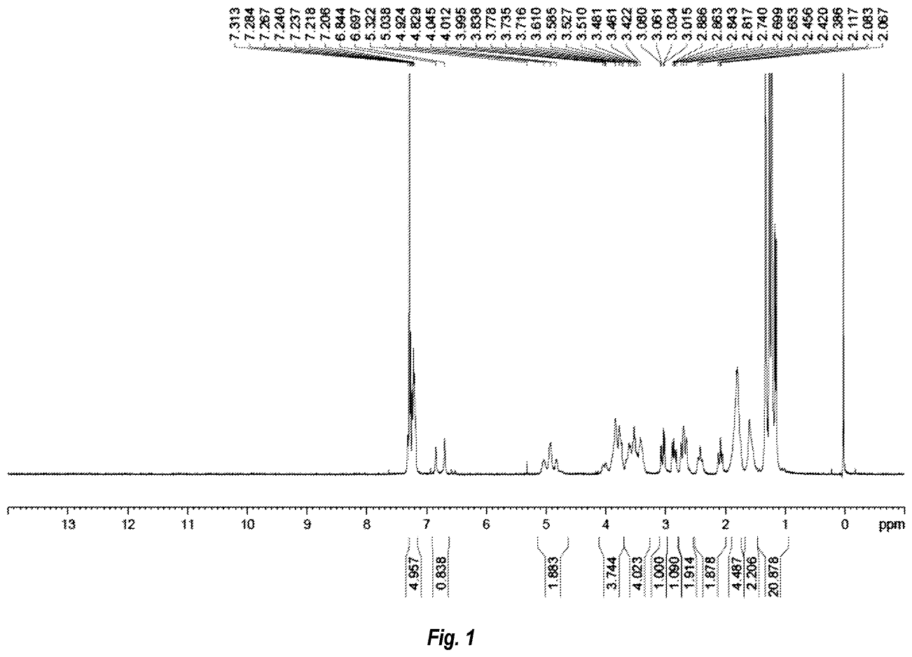

The pinacol ester of ((R)-1-(((((R)-1-(2-cyano-4-methyl-4-((R)-2-methylmorpholino)pent-2-enoyl)azepan-3-yl)oxy)carbonyl)amino)-2-phenylethyl)boronic acid is prepared by combining Boron Acid 1 with pinacol in dichloromethane. The reaction is stirred at ambient temperature, then is washed with water. The layers are separated, and the organic phase is concentrated to dryness to afford Ester N. Predicted MS [M+1]: 609. An exemplary NMR spectrum is shown in FIG. 1.

### Example 3: Preparation of Citric Acid Esters of [(1R)-1-[([[7-(2-cyano-2-[2-methyl-2-[(2S)-2-methylmorpholin-4-yl]propylidene]acetyl)-7-azabicyclo[2.2.1]heptan-1-yl]methoxy]carbonyl)amino]-2-phenylethyl]boronic acid

### Step 1: Synthesis of Intermediate 2

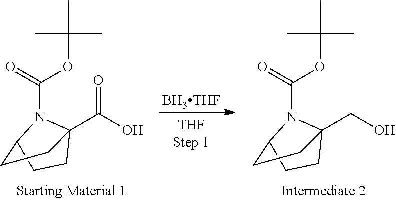 [SMILES: CC(C)(C)OC(=O)N1C2CCC1(C(=O)O)CC2 ; CC(C)(C)OC(=O)N1C2CCC1(CO)CC2 ; "BH3·THF"]

Into a 100-mL round-bottom flask, was placed Starting Material 1 (1 g, 4.144 mmol, 1 equiv.), oxolane (THF, 10 mL). This was followed by the addition of BH₃. THF (1M) (16.6 mL 4.0 equiv.) dropwise with stirring at 0° C. The resulting solution was stirred for 1 overnight at room temperature. The reaction was then quenched by the addition of 20 mL of MeOH at 0° C. The resulting mixture was concentrated under vacuum. The residue was applied onto a silica gel column with ethyl acetate/petroleum ether (1:20). This resulted in 900 mg (95.54%) of Intermediate 2 as a colorless oil.

### Step 2: Synthesis of Intermediate 3

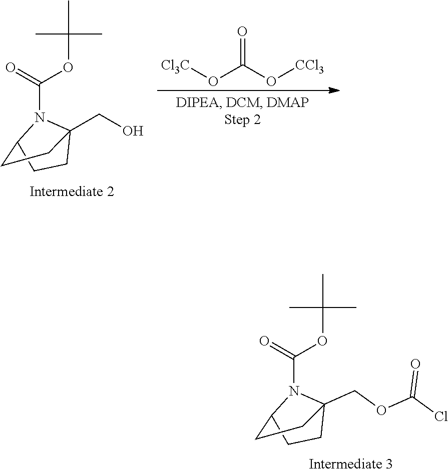

Into a 100-mL 3-necked round-bottom flask purged and maintained with an inert atmosphere of nitrogen, was placed Intermediate 2 (470 mg, 2.068 mmol, 1 equiv.), DCM (10 mL), DIPEA (801.71 mg, 6.203 mmol, 3.00 equiv.). This was followed by the addition of a solution of ditrichloromethyl carbonate (490.84 mg, 1.654 mmol, 0.80 equiv.) in DCM (2 mL) dropwise with stirring at 0° C. To this was added a solution of DMAP (126.30 mg, 1.034 mmol, 0.50 equiv.) in DCM (1 mL) at 0° C. The resulting solution was stirred for 2 hr at 0° C. in an ice/salt bath. The reaction mixture solution was used directly to the next step.

### Step 3: Synthesis of Intermediate 4

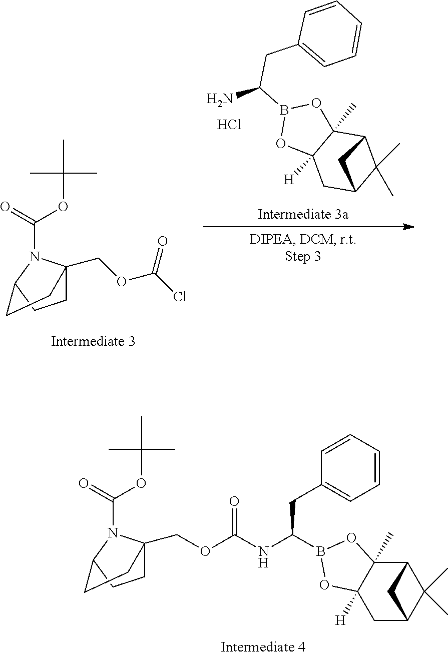 [SMILES: CC(C)(C)OC(=O)N1C2CCC1(COC(=O)Cl)CC2 ; Cl ; CC1(C)[C@@H]2C[C@H]3OB([C@@H](N)Cc4ccccc4)O[C@@]3(C)[C@H]1C2 ; CC(C)(C)OC(=O)N1C2CCC1(COC(=O)N[C@@H](Cc1ccccc1)B1O[C@@H]3C[C@@H]4C[C@@H](C4(C)C)[C@]3(C)O1)CC2]

Into a 100-mL 3-necked round-bottom flask purged and maintained with an inert atmosphere of nitrogen, was placed Intermediate 3a (683.50 mg, 2.036 mmol, 1.00 equiv.), DCM (10 mL), and DIPEA (789.48 mg, 6.109 mmol, 3.00 equiv.). This was followed by the addition of a solution of Intermediate 3 (590 mg, 2.036 mmol, 1 equiv.) in DCM (10 mL) dropwise with stirring at 0° C. The resulting solution was stirred for 1 hr at room temperature. The resulting mixture was washed with 1×30 ml of water. The mixture was dried over anhydrous sodium sulfate and concentrated under vacuum. The crude product was purified by Flash-Prep-HPLC with the following conditions: Column, C18 silica gel; mobile phase, ACN/H₂O=5/95 increasing to ACN/H₂O=100/0 within 45 min; Detector, 220 nm. This resulted in 700 mg (62.22%) of Intermediate 4 as a light yellow solid.

### Step 4: Synthesis of Intermediate 5

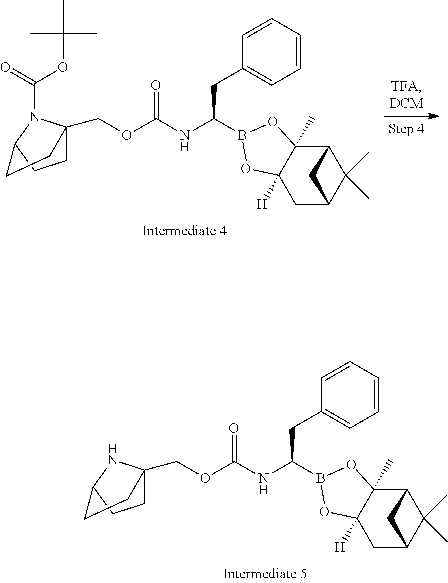 [SMILES: CC(C)(C)OC(=O)N1C2CCC1(COC(=O)N[C@@H](Cc1ccccc1)B1O[C@@H]3C[C@@H]4C[C@@H](C4(C)C)[C@]3(C)O1)CC2 ; CC1(C)[C@@H]2C[C@H]3OB([C@H](Cc4ccccc4)NC(=O)OCC45CCC(CC4)N5)O[C@@]3(C)[C@H]1C2]

Into a 250-mL round-bottom flask, was placed Intermediate 4 (1.434 g, 2.595 mmol, 1 equiv.), DCM (30 mL), and TFA (5 mL). The resulting solution was stirred for 1 hr at room temperature. The resulting mixture was concentrated under vacuum. This resulted in 1.15 g (97.94%) of Intermediate 5 as yellow oil.

### Step 5: Synthesis of Intermediate 6

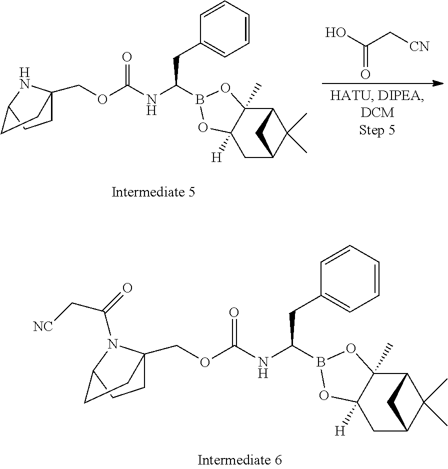 [SMILES: CC1(C)[C@@H]2C[C@H]3OB([C@H](Cc4ccccc4)NC(=O)OCC45CCC(CC4)N5)O[C@@]3(C)[C@H]1C2 ; N#CCC(=O)O ; CC1(C)[C@@H]2C[C@H]3OB([C@H](Cc4ccccc4)NC(=O)OCC45CCC(CC4)N5C(=O)CC#N)O[C@@]3(C)[C@H]1C2]

Into a 250-mL round-bottom flask, was placed Intermediate 5 (1.15 g, 2.542 mmol, 1 equiv.), DCM (30 mL), DIPEA (1.31 g, 10.136 mmol, 3.99 equiv.), 2-cyanoacetic acid (0.43 g, 5.055 mmol, 1.99 equiv.), and HATU (2.90 g, 7.627 mmol, 3.00 equiv.). The resulting solution was stirred for 1 hr at room temperature. The resulting mixture was washed with 1×30 mL of water. The mixture was dried over anhydrous sodium sulfate and concentrated under vacuum. The crude product was purified by Flash-Prep-HPLC with the following conditions: Column, C18 silica gel; mobile phase, ACN/H₂O=5/95 increasing to ACN/H₂O=90/10 within 45 min; Detector, 220 nm. This resulted in 900 mg (68.16%) of Intermediate 6 as a yellow solid.

Step 6: Synthesis of 2-bromo-2-methylpropanal

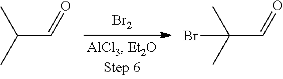

Into a 1L 3-necked round-bottom flask, was placed 2-methylpropanal (50 g, 693.43 mmol, 1.00 equiv.), ether (500 mL), and AlCl₃ (2.49 g, 0.03 equiv.). This was followed by the addition of dibromide (131.34 g, 821.86 mmol, 1.20 equiv.) dropwise with stirring at 0° C. in 10 min. The resulting solution was stirred overnight at room temperature. The reaction was then quenched by the addition of 100 mL of water/ice. The pH value of the solution was adjusted to 10-12 with sodium carbonate. The resulting solution was extracted with 3×100 mL of ether and the organic layers were combined and dried over anhydrous sodium sulfate and concentrated under vacuum. The crude product was purified by distillation under reduced pressure (170 mm Hg) and the fraction was collected at 70-77° C. This resulted in 21.0 g (20.0%) of 2-bromo-2-methylpropanal as a colorless oil.

Step 7: Synthesis of 2-methyl-2-[(2S)-2-methylmorpholin-4-yl]propanal

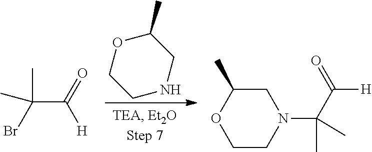

Into a 50-mL round-bottom flask, was placed (2S)-2-methylmorpholine (500 mg, 4.943 mmol, 1 equiv.), ethoxyethane (10 mL), 2-bromo-2-methylpropanal (1119.66 mg, 7.415 mmol, 1.5 equiv.), and TEA (1500.61 mg, 14.830 mmol, 3.00 equiv.). The resulting solution was stirred for 3 days at room temperature. The resulting mixture was washed with 1×10 mL of water. The mixture was dried over anhydrous sodium sulfate and concentrated under vacuum. The residue was applied onto a silica gel column with ethyl acetate/hexane (1:10). This resulted in 600 mg (70.88%) of 2-methyl-2-[(2S)-2-methylmorpholin-4-yl]propanal as yellow oil.

### Step 8: Synthesis of Intermediate 7

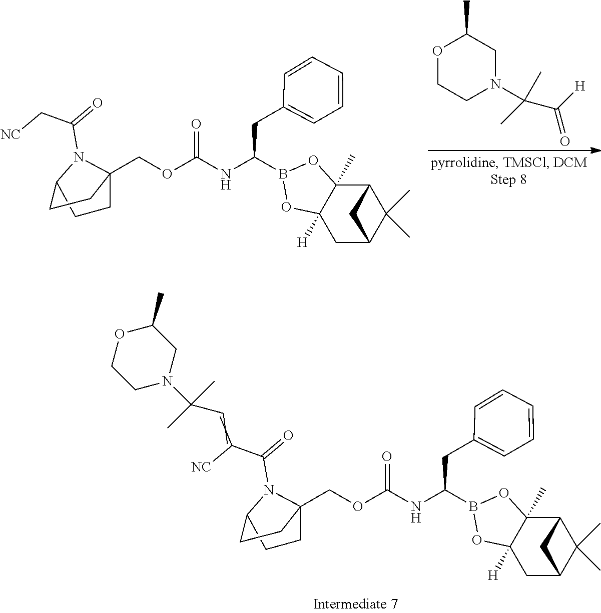 [SMILES: CC1(C)[C@@H]2C[C@H]3OB([C@H](Cc4ccccc4)NC(=O)OCC45CCC(CC4)N5C(=O)CC#N)O[C@@]3(C)[C@H]1C2 ; C[C@H]1CN(C(C)(C)C=O)CCO1 ; C[C@H]1CN(C(C)(C)C=C(C#N)C(=O)N2C3CCC2(COC(=O)N[C@@H](Cc2ccccc2)B2O[C@@H]4C[C@@H]5C[C@@H](C5(C)C)[C@]4(C)O2)CC3)CCO1]

Into a 50-mL round-bottom flask, was placed Intermediate 6 (170 mg, 0.327 mmol, 1 equiv.), DCM (5 mL), 2-methyl-2-[(2S)-2-methylmorpholin-4-yl]propanal (168.12 mg, 0.982 mmol, 3.00 equiv.), pyrrolidine (116.38 mg, 1.636 mmol, 5.00 equiv.), and TMSCl (177.78 mg, 1.636 mmol, 5.00 equiv.). The resulting solution was stirred for 1 hr at room temperature. The resulting mixture was washed with 1×10 mL of water. The mixture was dried over anhydrous sodium sulfate and concentrated under vacuum. The crude product was purified by Flash-Prep-HPLC with the following conditions: Column, C18 silica gel; mobile phase, ACN/H₂O=5/95 increasing to ACN/H₂O=100/0 within 45 min; Detector, 220 nm. This resulted in 140 mg (63.59%) of Intermediate 7 as a light yellow solid.

### Step 9: Synthesis of Boronic Acid 2

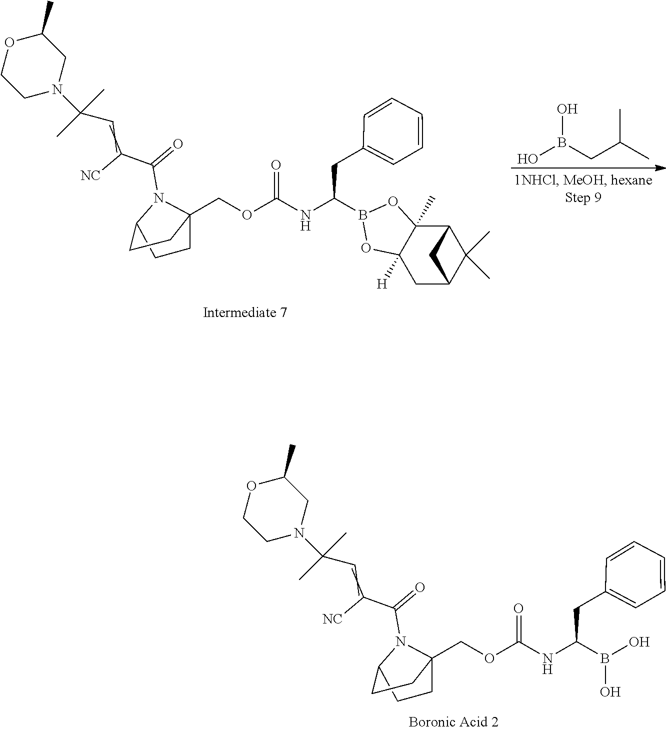

Into a 100-mL round-bottom flask, was placed Intermediate 7 (140 mg, 0.208 mmol, 1 equiv.), MeOH (4 mL), (2-methylpropyl)boronic acid (63.65 mg, 0.624 mmol, 3.00 equiv.), hexane (4 mL), and 1N HCl (4 mL). The resulting solution was stirred for 1 hr at room temperature. The methanol layer was diluted with 25 mL of water and dried over lyophilization to give a crude product. The crude product was purified by Prep-HPLC with the following conditions: Column, XBridge Prep OBD C18 Column, 30*150 mm 5 μm; mobile phase, Water (10MMOL/L NH₄HCO₃) and ACN (25% Phase B up to 55% in 8 min); Detector, UV 254/220 nm. This resulted in 39.2 mg (34.98%) of Boronic Acid 2 as a white solid. LC-MS m/z: 539 (M+1); 1H NMR (300 MHz, Methanol-d4) δ 7.23 (m, 5H), 7.03 (s, 1H), 4.75 (s, 2H), 4.42 (s, 1H), 3.79 (m, 3H), 3.24 (t, J=7.6 Hz, 1H), 2.92-2.64 (m, 4H), 2.48-2.35 (m, 1H), 2.15-2.02 (m, 1H), 1.91 (m, 4H), 1.73-1.60 (m, 4H), 1.40-1.22 (m, 6H), 1.12 (m, 3H).

### Step 10: Synthesis of Citric Acid Esters of Boronic Acid 2

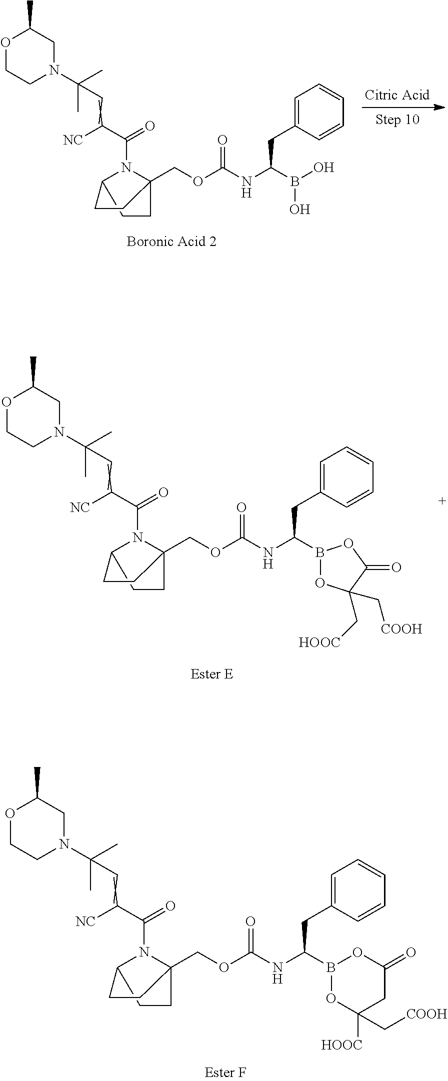 [SMILES: C[C@H]1CN(C(C)(C)C=C(C#N)C(=O)N2C3CCC2(COC(=O)N[C@@H](Cc2ccccc2)B(O)O)CC3)CCO1 ; C[C@H]1CN(C(C)(C)C=C(C#N)C(=O)N2C3CCC2(COC(=O)N[C@@H](Cc2ccccc2)B2OC(=O)C(CC(=O)O)(CC(=O)O)O2)CC3)CCO1 ; C[C@H]1CN(C(C)(C)C=C(C#N)C(=O)N2C3CCC2(COC(=O)N[C@@H](Cc2ccccc2)B2OC(=O)CC(CC(=O)O)(C(=O)O)O2)CC3)CCO1]

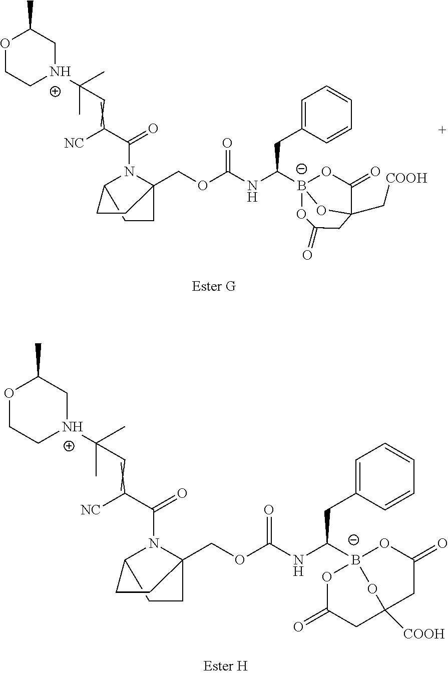 [SMILES: C[C@H]1C[NH+](C(C)(C)C=C(C#N)C(=O)N2C3CCC2(COC(=O)N[C@@H](Cc2ccccc2)[B-]24OC(=O)CC(CC(=O)O)(O2)C(=O)O4)CC3)CCO1 ; C[C@H]1C[NH+](C(C)(C)C=C(C#N)C(=O)N2C3CCC2(COC(=O)N[C@@H](Cc2ccccc2)[B-]24OC(=O)CC(C(=O)O)(CC(=O)O2)O4)CC3)CCO1]

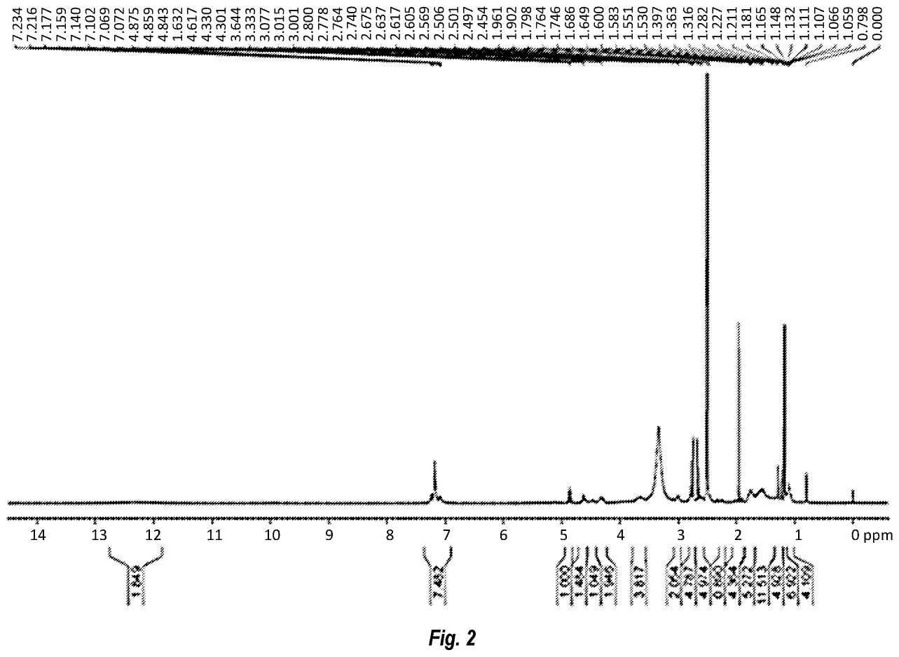

To prepare the citric acid esters, a solution of anhydrous citric acid (345 mg, 1.0 eq.) in EtOH (5.0 mL) was added to a solution of Boronic Acid 2 (1.0 g) in dichloromethane (20 mL) at 15-20° C. The solution was concentrated at a temperature lower than 18° C. under vacuum to 5-6 mL. The concentrated solution was then added to n-Heptane (30.0 mL) slowly resulting in a slurry which was stirred for 30 min. The resulting solid was filtered and washed with n-Heptane (2 mL) and dried under vacuum to give a mixture of citric acid boron esters (Esters E-H) (970 mg, 75% yield with 97.1% purity by HPLC). MS [M+1]: 695, 330 (SFC-MS). NMR is shown in FIG. 2.

### Example 4: Preparation of a Solvate-Citric Acid Ester Crystal [(1R)-1-[([[7-(2-cyano-2-[2-methyl-2-[(2S)-2-methylmorpholin-4-yl]propylidene]acetyl)-7-azabicyclo[2.2.1]heptan-1-yl]methoxy]carbonyl)amino]-2-phenylethyl]boronic acid

A solvate-citric acid ester crystal of [(1R)-1-[([7-(2-cyano-2-[2-methyl-2-[(2S)-2-methylmorpholin-4-yl]propylidene]acetyl)-7-azabicyclo[2.2.1]heptan-1-yl]methoxy]carbonyl)amino]-2-phenylethyl]boronic acid was obtained by reaction crystallization of Boronic Acid 2 with 1.1 equivalents of citric acid in acetonitrile, or by the reactive crystallization of Boronic Acid 2 with 1.1 equivalents of citric acid in a mixture of EtOH/ACN (1:1 or 1:2). The reaction crystallizations were followed by cooling to 0-10° C., resulting in a wet cake that showed good crystallinity. SFC/MS of the wet cake showed the [(1R)-1-[([7-(2-cyano-2-[2-methyl-2-[(2S)-2-methylmorpholin-4-yl]propylidene]acetyl)-7-azabicyclo[2.2.1]heptan-1-yl]methoxy]carbonyl)amino]-2-phenylethyl]boronic acid citric acid ester peak of [M+1] of 695. While the wet cake showed good crystallinity, its crystallinity decreased sharply when it was air dried for a few minutes and became amorphous.

### Example 5: Preparation of Citric Acid Esters of ((R)-1-(((((R)-1-(2-cyano-4-(3,3-difluoropyrrolidin-1-yl)-4-methylpent-2-enoyl)piperidin-2-yl)methoxy)carbonyl)amino)-2-phenylethyl)boronic acid

### Steps 1 and 2: Synthesis of Intermediates 1 and 2

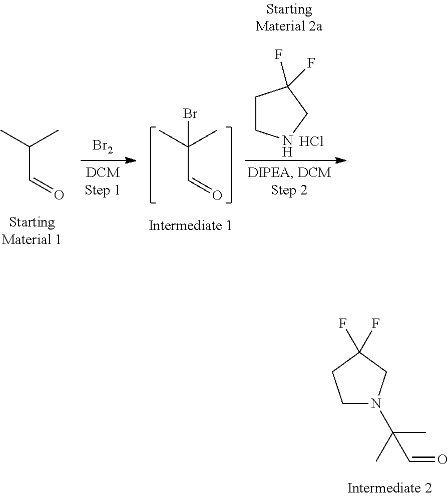

The reaction was performed in a 3 necked-round bottom flask under N₂. Bromine was added dropwise to a solution of Starting Material 1 (isobutyraldehyde) in DCM with magnetic stirring. The reaction was stirred for at least 15 mins at 0±5° C. The sample was taken for GC analysis. The solution was then washed with Na₂SO₃ aqueous solution, saturated NaHCO₃ aqueous and brine. The solution was used in the next step without further purification.

The reaction was performed in a 3 necked-round bottom flask under N₂. The solution from Step 1 was added dropwise with magnetic stirring to a solution of 3,3-difluoropyrrolidine hydrochloride (Starting Material 2a) and DIPEA in DCM. The reaction was allowed to react for at least 17 hours at 0±5° C. and monitored by GC analysis and work up and concentrate to a syrup.

### Step 3: Synthesis of Intermediate 3

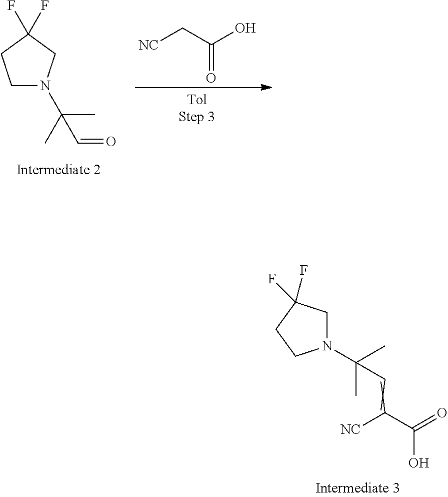 [SMILES: CC(C)(C=O)N1CCC(F)(F)C1 ; N#CCC(=O)O ; CC(C)(C=C(C#N)C(=O)O)N1CCC(F)(F)C1]

The reaction was run in a bottom flask under N₂. Intermediate 2 in toluene, 2-cyanoacetic acid and NH₄OAc were added in sequence to the reaction flask with magnetic stirring. The reaction was stirred at 80±5° C. for at least 48 hours. The sample was analyzed by GC and reaction progress was monitored by the ratio of Intermediate 2/solvent.

### Step 4: Synthesis of Intermediate 4

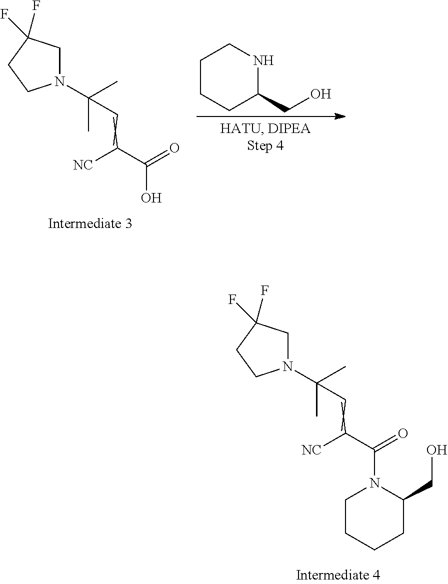 [SMILES: CC(C)(C=C(C#N)C(=O)O)N1CCC(F)(F)C1 ; OC[C@H]1CCCCN1 ; CC(C)(C=C(C#N)C(=O)N1CCCC[C@@H]1CO)N1CCC(F)(F)C1]

The reaction was performed in a 3 necked-round bottom flask under N₂. Intermediate 3 in DCM and (R)-piperidin-2-ylmethanol, DIPEA and HATU were added in sequence to the reaction flask with magnetic stirring. The reaction was stirred for at least 4 h at 0±5° C. Reaction was monitored by LC/MS.

### Step 5: Synthesis of Intermediate 5

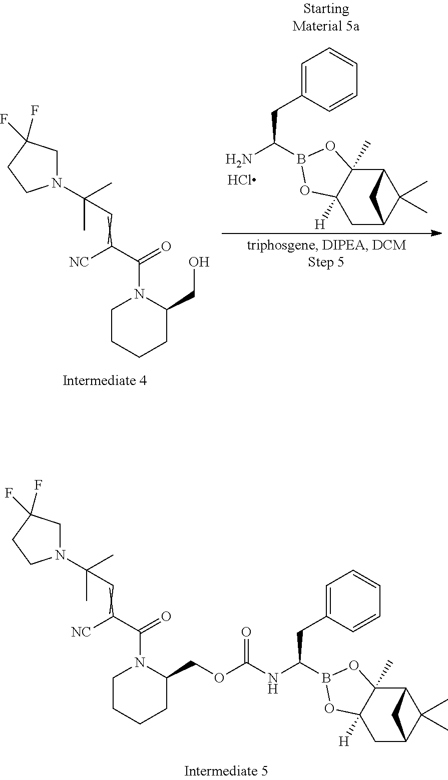 [SMILES: CC(C)(C=C(C#N)C(=O)N1CCCC[C@@H]1CO)N1CCC(F)(F)C1 ; CC1(C)[C@@H]2C[C@H]3OB([C@@H](N)Cc4ccccc4)O[C@@]3(C)[C@H]1C2 ; CC1(C)[C@@H]2C[C@H]3OB([C@H](Cc4ccccc4)NC(=O)OC[C@H]4CCCCN4C(=O)C(C#N)=CC(C)(C)N4CCC(F)(F)C4)O[C@@]3(C)[C@H]1C2]

The product from Step 4 (20.0 g) and dichloromethane (250 mL) were charged to a 1000 mL jacketed reactor equipped with an overhead mechanical stirrer. The solution was cooled to 0±5° C., followed by the addition of DIPEA (31.5 g). To the reaction was added triphosgene (12.2 g) in dichloromethane (60 mL) dropwise, maintaining an internal temperature of 0±5° C. The reaction was stirred for 20 minutes at 0±5° C. to give solution A. To a separate reactor was added Starting Material 5a (19.07 g) and dichloromethane (300 mL). The mixture was stirred and cooled to −5±5° C., followed by the addition of DIPEA (15.14 g) at −5± 5° C. To the stirred reaction was added solution A dropwise, maintaining an internal temperature of −5 to 0° C. The reaction was stirred for 5 minutes and then quenched with water (200 mL) at −5 to 0° C. The mixture was stirred from 10 minutes at −5 to 0° C., then the phases were allowed to separate. The organic phase was washed with water (600 mL). To the organic phase was added cool water (300 mL) and the pH was adjusted to 2-3 with 1N HCl, maintaining an internal temperature of 5±5° C. The organic phase was separated and washed with cool water (300 mL). The organic phase was concentrated to 150 mL, followed by the addition of methanol (900 mL). The solution was concentrated under reduced pressure at <15° C. to 750 mL.

### Step 6: Synthesis of Boronic Acid 3

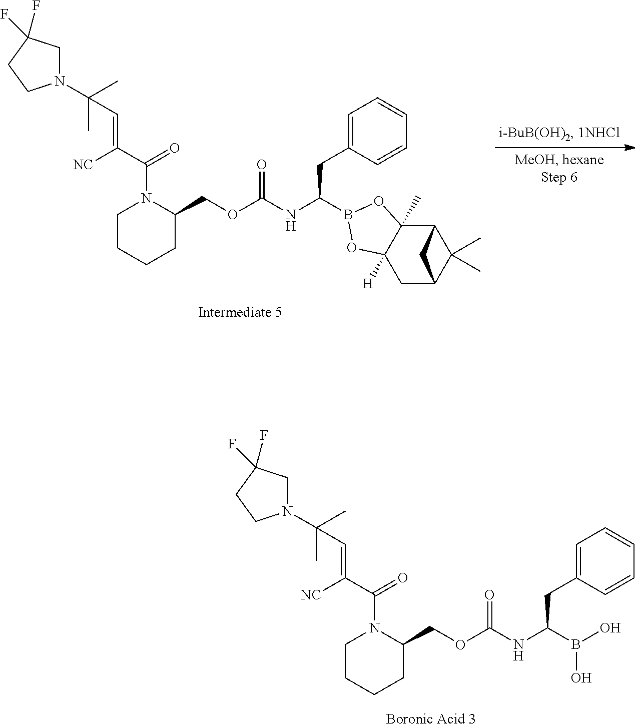 [SMILES: CC1(C)[C@@H]2C[C@H]3OB([C@H](Cc4ccccc4)NC(=O)OC[C@H]4CCCCN4C(=O)/C(C#N)=C/C(C)(C)N4CCC(F)(F)C4)O[C@@]3(C)[C@H]1C2 ; CC(C)(/C=C(\C#N)C(=O)N1CCCC[C@@H]1COC(=O)N[C@@H](Cc1ccccc1)B(O)O)N1CCC(F)(F)C1]

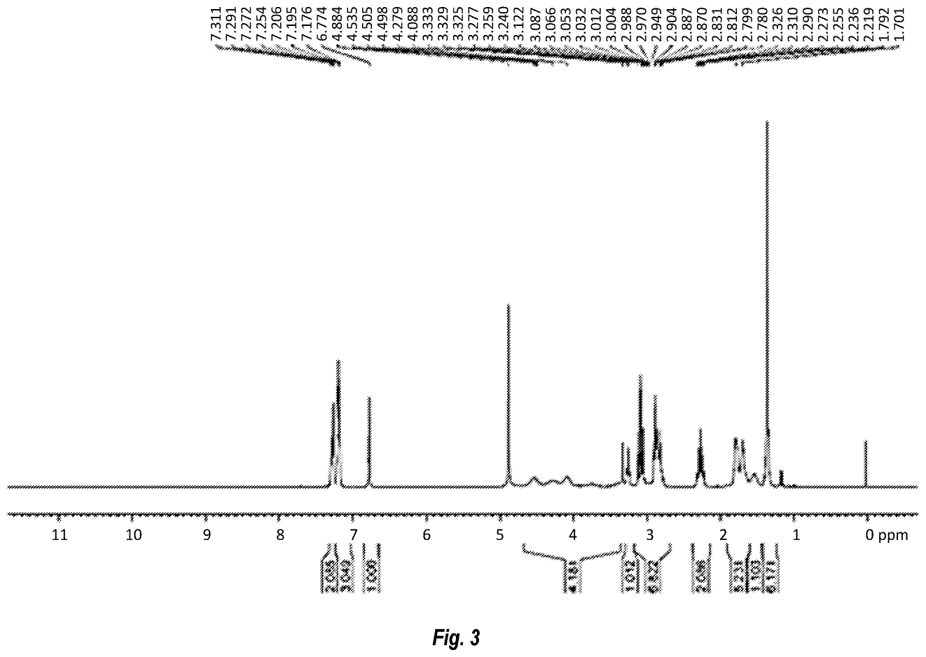

To the solution from Step 5 (750 mL) was added n-heptane (750 mL). To the mixture was added 1 N HCl (600 mL) at 5±5° C., followed by the addition of i-BuB (OH) 2 (2.0 eq.) in MeOH (60 mL) dropwise at 5±5° C. The reaction was stirred for 2 hours. The phases were separated and the aqueous phase was washed with n-heptane (750 mL) three times. Water (600 mL) was added to the aqueous phase and extracted with dichloromethane (600 mL), followed by a second extraction with dichloromethane (300 mL). The dichloromethane extractions were combined and washed with water (600 mL) twice. The dichloromethane solution was directly purified by silica gel chromatography, eluting with dichloromethane and isopropanol. The fractions containing the product were combined and washed with water (30 volumes) five times. The resulting organic solution was concentrated under reduced pressure below 15° C. to ~30 V, and then solvent swapped to water/acetonitrile (600 mL, 1:1). Boronic Acid 3 (23.0 g) was obtained after lyophilization as a white powder. The NMR is shown in FIG. 3.

### Step 7: Synthesis of Citric Acid Esters of Boronic Acid 3

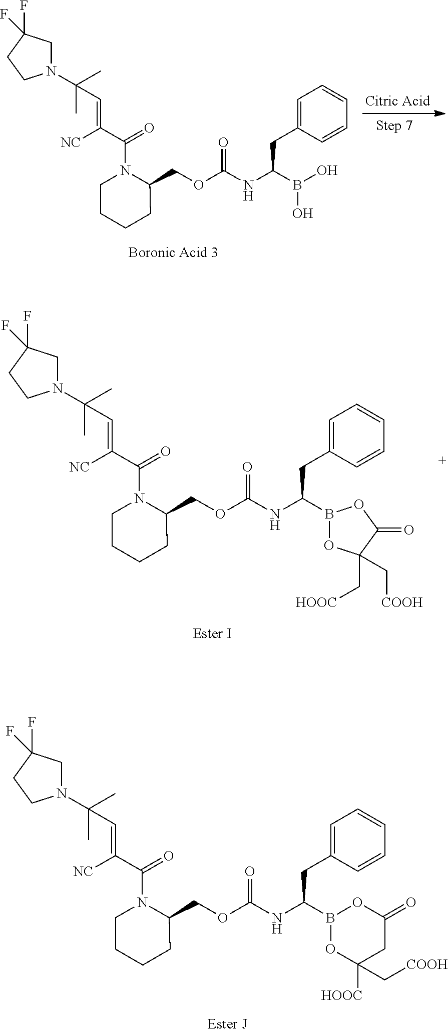 [SMILES: CC(C)(/C=C(\C#N)C(=O)N1CCCC[C@@H]1COC(=O)N[C@@H](Cc1ccccc1)B(O)O)N1CCC(F)(F)C1 ; CC(C)(/C=C(\C#N)C(=O)N1CCCC[C@@H]1COC(=O)N[C@@H](Cc1ccccc1)B1OC(=O)C(CC(=O)O)(CC(=O)O)O1)N1CCC(F)(F)C1 ; CC(C)(/C=C(\C#N)C(=O)N1CCCC[C@@H]1COC(=O)N[C@@H](Cc1ccccc1)B1OC(=O)CC(CC(=O)O)(C(=O)O)O1)N1CCC(F)(F)C1]

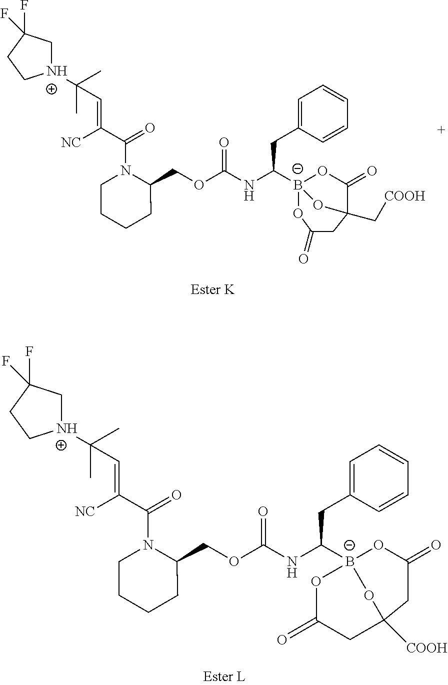 [SMILES: CC(C)(/C=C(\C#N)C(=O)N1CCCC[C@@H]1COC(=O)N[C@@H](Cc1ccccc1)[B-]12OC(=O)CC(CC(=O)O)(O1)C(=O)O2)[NH+]1CCC(F)(F)C1 ; CC(C)(/C=C(\C#N)C(=O)N1CCCC[C@@H]1COC(=O)N[C@@H](Cc1ccccc1)[B-]12OC(=O)CC(C(=O)O)(CC(=O)O1)O2)[NH+]1CCC(F)(F)C1]

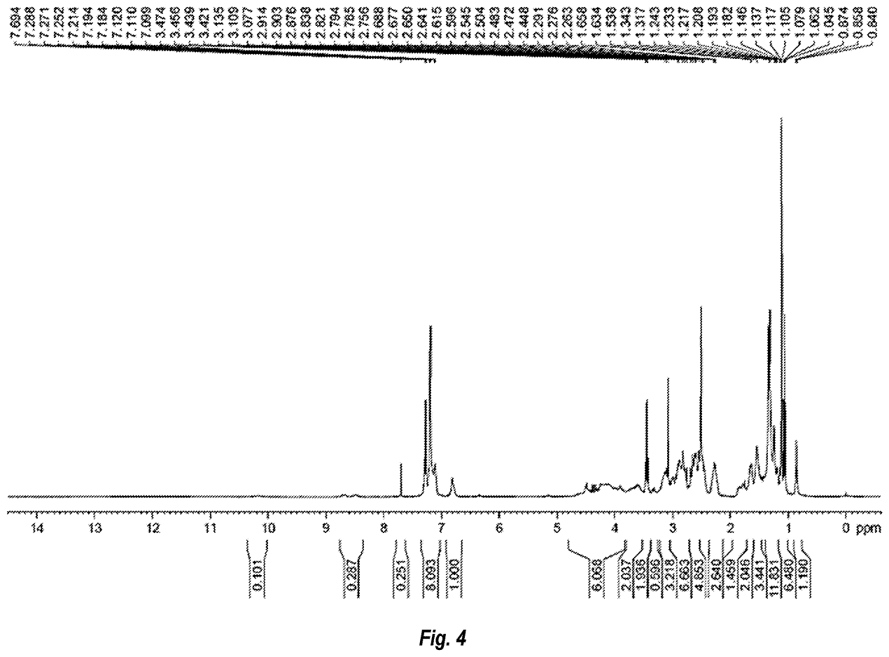

To prepare the citric acid esters, a solution of anhydrous citric acid (379 mg, 1.05 eq.) in ethanol (EtOH, 2.5 mL) was charged to a solution of Boronic Acid 3 (1.0 g) in EtOH. The mixture was stirred for 20 min at 15-20° C. Methyl t-butyl ether (MTBE, 40 mL) was added to the solution and a solid was formed. After stirring for 1 h at 20° C., the slurry was filtered and the wet cake was washed with MTBE (2 mL). After drying at room temperature under vacuum, a mixture of citric acid esters (Esters I-L) was obtained as an off-white solid (970 mg, 75% yield with 84.6% purity by HPLC). MS [M+1]: 689 (SFC-MS). The NMR is shown in FIG. 4.

### Example 6: Preparation of a Salicylic Acid Ester of ((R)-1-(((((R)-1-(2-cyano-4-(3,3-difluoropyrrolidin-1-yl)-4-methylpent-2-enoyl)piperidin-2-yl)methoxy)carbonyl)amino)-2-phenylethyl)boronic acid

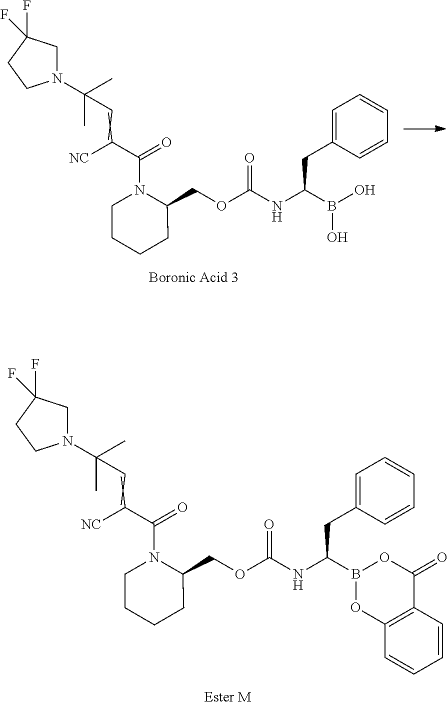 [SMILES: CC(C)(C=C(C#N)C(=O)N1CCCC[C@@H]1COC(=O)N[C@@H](Cc1ccccc1)B(O)O)N1CCC(F)(F)C1 ; CC(C)(C=C(C#N)C(=O)N1CCCC[C@@H]1COC(=O)N[C@@H](Cc1ccccc1)B1OC(=O)c2ccccc2O1)N1CCC(F)(F)C1]

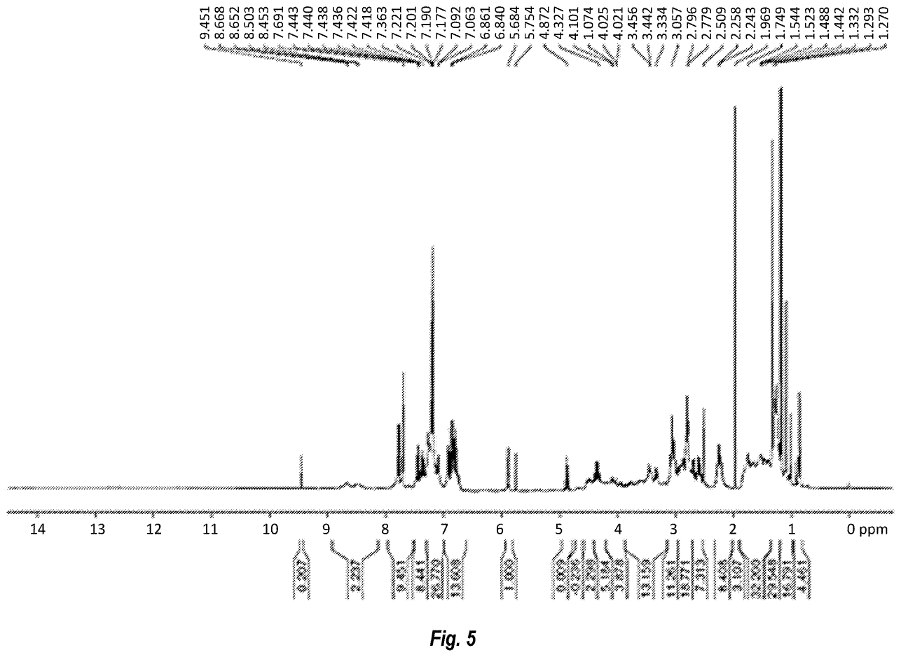

The salicylic ester of ((R)-1-(((((R)-1-(2-cyano-4-(3,3-difluoropyrrolidin-1-yl)-4-methylpent-2-enoyl)piperidin-2-yl)methoxy)carbonyl)amino)-2-phenylethyl)boronic acid can be prepared by charging Boronic Acid 3 (500 mg) in isopropylacetate (2.5 mL) and adding a solution of salicylic acid (1.1 eq.) in isopropylacetate (1.25 mL). The resulting mixture was stirred for 2 h at 20-25° C. To the reaction was added n-heptane (25 mL), forming a solid precipitate, which was stirred for 30 minutes at 20-25° C. The solid was filtered and rinsed with n-heptane (1 mL) and dried with nitrogen on the filter to afford 610 mg of the salicylate ester as a yellow solid. MS [M+1]: 635 (SFC-MS). The NMR is shown in FIG. 5.

### Example 7: Polymorphs of the Citric Acid Esters of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid

Form I—Form I was obtained by slurrying Form II in EtOH and IPA at RT and 50° C. It could also be formed by slurrying Form III with EtOH at room temperature and at 50° C. Form I was found to be stable at 40° C./75% relative humidity and 60° C. for 7 days. The prepared Form I was a plate crystal with good crystallinity (FIG. 6). It showed 0.5% of weight loss on TGA profile and an endothermic peak at 179/193° C. on DSC profile (FIG. 7). The characterization data indicated it was an anhydrate.

Form II—Form II is a solvate with acetonitrile. Form II was prepared from the HCl of Boronic Acid 1. The HCl salt was prepared by dissolving Boronic Acid 1 (140g) in EtOAc (10 V) and washing with NaOH (0.2 eq) in water (5 V). The layers were then separated and the organic phase was charged with 10% brine (5 V). The pH was adjusted to ~6-7 with 1N HCl. The layers were again separated and the organic phase was dried with Na₂SO₄, filtered and the cake was washed with EtOAc (2V). The solution was concentrated and redissolved in DCM (5 V) under less than 20° C. The reaction was charged with HCl (1 eq.) in EtOAc and stirred at ~20° C. for 30 min. The solution was charged with MTBE (20 V) and DCM (5 V) at 20° C. and stirred at ~20° C. for ~2 hours. The mixture was filtered and the cake was washed with MTBE/DCM=2/1 (2 V). The cake was slurried with EtOAc (10 V) and MTBE (10 V) at ~20° C. and then stirred for ~2 hrs at ~20° C. The organic layer was filtered and the cake was washed with EtOAc/MTBE=1/1 (2 V). The cake was dried under vacuum at ~20° C. for 4 hours to afford the HCl salt as a light yellow solid.

The HCl salt (8g) was then added to DCM (160 mL) and the mixture was charged with H₂O (10 V). The pH was adjusted to ~7 with 9% NaHCO₃. The layers were separated and the organic phase was washed once with brine. The DCM solution was dried with Na₂SO₄, concentrated and swapped with EtOH (44 mL) under vacuum to give Boronic Acid 1. Citric acid (3.14g) in EtOH solution (80 mL) was added to the Boronic Acid 1 and the system was charged with n-heptane (32 mL) resulting in a muddy solution. The mixture was stirred at ~15-20° C. overnight (at least 16 hours). The reaction was then charged with n-heptane (88 mL) and IPAc (16 mL) and stirred for ~3 hours. The reaction was filtered and the cake was washed with EtOH/n-heptane (1:2). The cake was dried under vacuum to give the citric acid ester, which was then recrystallized in MeCN. More specifically, the citric acid ester (8.3g) was added to MeCN (830 mL, 10 V) and stirred at ~40-45° C. for ~2 hours before cooling to ~15-20° C. over ~3 hours at least. The reaction was charged with additional MeCN (5 V) and MTBE (3 V) and stirred at ~40-45° C. for ~20 hours. The solution was filtered and the cake was washed with MeCN/MTBE (5:1, 1 V). The cake was dried under vacuum to afford Form II. The XRPD is shown in FIG. 8. The TGA and DSC thermograms are shown in FIG. 9.

Form III—Form III is a hydrate formed by dehydration of Form II. Form III was also obtained by slurrying Form II in MTBE, toluene, EA and IPAc at room temperature or at ~50° C. The XRPD is shown in FIG. 10. The TGA and DSC thermograms are shown in FIG. 11.

Form IV—Form IV was a hydrate formed by slurrying Form II in MEK at room temperature. The XRPD is shown in FIG. 12. The TGA and DSC thermograms are shown in FIG. 13.

Form V—Form V was a hydrate obtained from Form IV, which first goes through a metastable form and then, after air drying for 20 minutes, transformed into Form V. Form V was also prepared by slurrying Form I in MEK at ~50° C./25° C. cycling (8 hr/cycle) for ~24 hours. The resulting suspension was filtered and dried briefly with compressed air to give Form V. The XRPD is shown in FIG. 14. The TGA and DSC thermograms are shown in FIG. 15.

### Example 8: Stability of Boronic Esters of ((R)-1-(((((R)-1-((E)-2-cyano-4-methyl-4-((R)-2-methylmorpholino)pent-2-enoyl)azepan-3-yl)oxy)carbonyl)amino)-2-phenylethyl) boronic acid

Stability in SGF and SIF: The stability of the citric acid ester of Boronic Acid 1 in SGF (Simulated Gastric Fluid, pH 1.2) and SIF (Simulated Intestinal Fluid, pH 6.8) was studied by diluting a 10 mg/mL solution of the ester in ACN with SGF or SIF to achieve a final ester concentration of 0.5 mg/mL. The diluted solution was then analyzed for ester content by UPLC. It was discovered that, when a solution of the citric acid ester of Boronic Acid 1 in ACN was added to SGF, the ester completely converted to Boronic Acid 1 immediately. When a solution of the citric acid ester of Boronic Acid 1 in ACN was added to SIF, the ester mostly converted to Boronic Acid 1 immediately with approximately 8% of the ester remaining.

The same set of experiments were performed for the pinacol and pinanediol esters of Boronic Acid 1 except that additional ACN was added when diluting into SIF in order to maintain the compounds in solution (final ACN concentration of 20%-30% in diluted solutions in SIF). It was found that when added to SGF and SIF, the pinacol ester of Boronic Acid 1 completely converted to Boronic Acid 1 immediately while the pinanediol ester (Intermediate 5 from Example 1) only partially converted to Boronic Acid 1 (with a ratio of Boronic Acid 1: Intermediate 5 of 65:35 in SGF and 37:63 in SIF).

Stability in Open Dish Experiments: An open dish stability study was performed at 40° C./75% RH in open glass vials to compare the stability of the citric acid ester of Boronic Acid 1 against Boronic Acid 1, the pinacol ester of Boronic Acid 1, the pinanediol ester of Boronic Acid 1 and the HCl salt of Boronic Acid 1.

The results (shown in the table below) showed that Boronic Acid 1, its HCl salt, and the pinacol and pinanediol esters of Boronic Acid 1 all significantly degraded after two weeks while the citric acid ester of Boronic Acid 1 was stable for at least four weeks. In addition, the citric acid ester demonstrated acceptable stability for at least 6 months at 5° C., 25° C./60% RH and 40° C./75% RH conditions.

TABLE 2

| | % Peak Area by UPLC | | |
|---|---|---|---|
| | | 40° C./75% RH open | |
| Compound | Initial | 2 weeks | 4 weeks |
| Boronic Acid 1 | 95.8 | 85.9 | Not tested |
| Boronic Acid 1 - HCl salt | 96.3 | 76.4 | Not tested |
| Pinacol ester of Boronic Acid 1 | 95.5 | 70.9 | Not tested |
| Pinanediol ester of Boronic Acid 1 | 93.5 | 90.6 | Not tested |
| Citric Acid ester of Boronic Acid 1 | 99.9 | 99.5 | 99.2 |

Discussion: Boronic Acid 1 is amorphous and is difficult to manufacture with high purity. By contrast, its corresponding citric acid ester is a crystalline material and has been manufactured with high purity (>99% area) on larger scale. Further, Boronic Acid 1 is prone to hydrolysis and oxidation. Due to the presence of the boronic acid functional groups in Boronic Acid 1, the molecule can easily be oxidized by peroxide and can even be oxidized by oxygen present in the environment or dissolved in the solution. This inherent chemical instability could lead to chemical instability during manufacturing and storage. The citric acid ester of Boronic Acid 1 has demonstrated much improved chemical stability as shown in the stability studies above. In addition, the citric acid readily converts to Boronic Acid 1 in aqueous media under physiological conditions (e.g., SGF and SIF).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby. The disclosures of all patent and scientific literature cited herein are expressly incorporated herein in their entirety by reference. To the extent that any incorporated material is inconsistent with the express content of this disclosure, the express content controls.

1. A compound of Formula (I), or a pharmaceutically acceptable salt thereof, 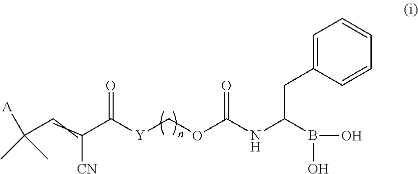
wherein
A is an optionally substituted mono- or bicyclic heterocyclic ring containing at least one nitrogen atom bond to the methylene unit;
Y is an optionally substituted mono- or bicyclic heterocyclic ring containing at least one nitrogen atom bonded to the carbonyl group;
X is a boronic ester, which is bound to the methylene unit through the boron atom and is chosen from a citric acid ester, a pinacol ester, a malic acid ester, a salicylic acid ester, a lactic acid ester, and a tartaric acid ester; and
n is 0 or 1.

2. The compound according to claim 1, wherein A is an optionally substituted mono- or bicyclic 5 or 6-membered heterocyclic ring.

3. The compound according to any one of the preceding claims, wherein A is chosen from an optionally substituted pyrrolidinyl and an optionally substituted morpholinyl.

4. The compound according to any one of the preceding claims, wherein A is chosen from:

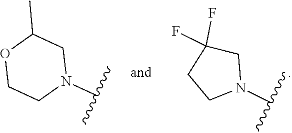

5. The compound according to any one of the preceding claims, wherein Y is an optionally substituted mono- or bicyclic 6 or 7-membered heterocyclic ring.

6. The compound according to any one of the preceding claims, wherein Y is chosen from piperidinyl, azepanyl, and 7-azabicyclo[2.2.1]heptan-7-yl, each of which is optionally substituted.

7. The compound according to any one of the preceding claims, wherein Y is chosen from:

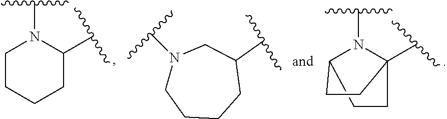 [SMILES: *C1CCCCN1* ; *C1CCCCN(*)C1 ; *N1C2CCC1(*)CC2]

8. The compound according to any one of the preceding claims, wherein X is a citric acid boronic ester.

9. The compound according to any one of the preceding claims, wherein X is chosen from:

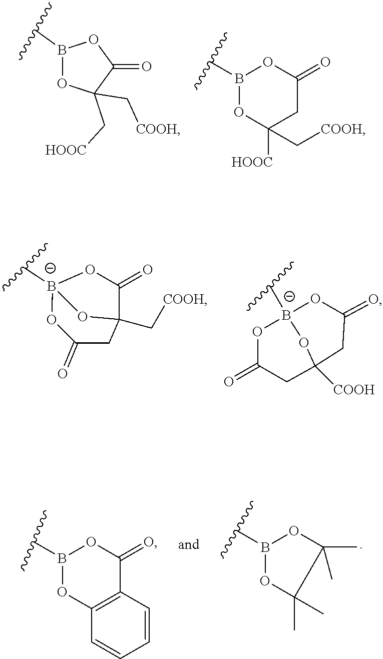

10. A compound prepared by reacting a compound of Formula (i) or a pharmaceutically acceptable salt thereof with a reagent chosen from citric acid, malic acid, salicylic acid, lactic acid, tartaric acid and pinacol, wherein Formula (i) is: 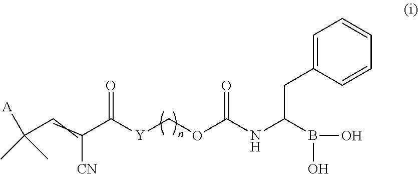
wherein,
A is an optionally substituted mono- or bicyclic heterocyclic ring containing at least one nitrogen atom bond to the methylene unit;
Y is an optionally substituted mono- or bicyclic heterocyclic ring containing at least one nitrogen atom bonded to the carbonyl group; and
n is 0 or 1.

11. The compound according to claim 10, wherein A is an optionally substituted mono- or bicyclic 5 or 6-membered heterocyclic ring.

12. The compound according to claim 10 or 11, wherein A is chosen from:

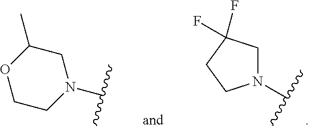

13. The compound according to any one of claims 10-12, wherein Y is an optionally substituted mono- or bicyclic 6 or 7-membered heterocyclic ring.

14. The compound according to any one of claims 10-13, wherein Y is chosen from

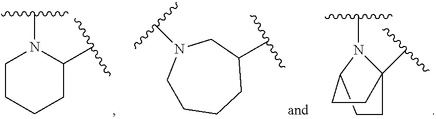 [SMILES: *C1CCCCN1* ; *C1CCCCN(*)C1 ; *N1C2CCC1(*)CC2]

15. The compound according to any one of claims 10-14, wherein the reagent is citric acid.

16. A compound chosen from:

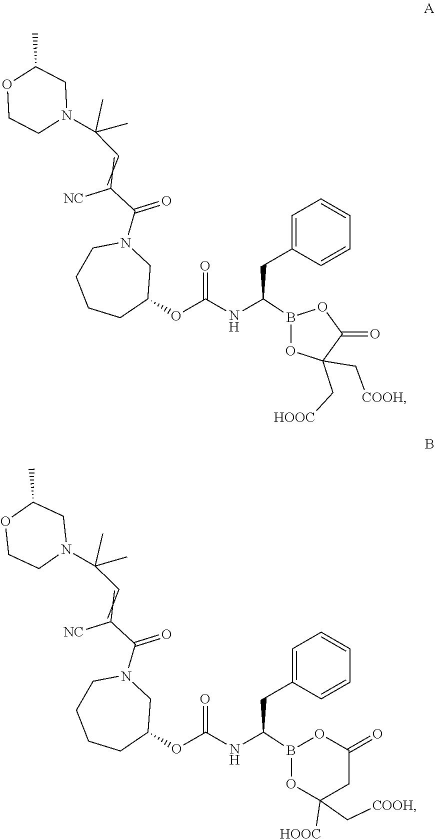 [SMILES: C[C@@H]1CN(C(C)(C)C=C(C#N)C(=O)N2CCCC[C@@H](OC(=O)N[C@@H](Cc3ccccc3)B3OC(=O)C(CC(=O)O)(CC(=O)O)O3)C2)CCO1 ; C[C@@H]1CN(C(C)(C)C=C(C#N)C(=O)N2CCCC[C@@H](OC(=O)N[C@@H](Cc3ccccc3)B3OC(=O)CC(CC(=O)O)(C(=O)O)O3)C2)CCO1]

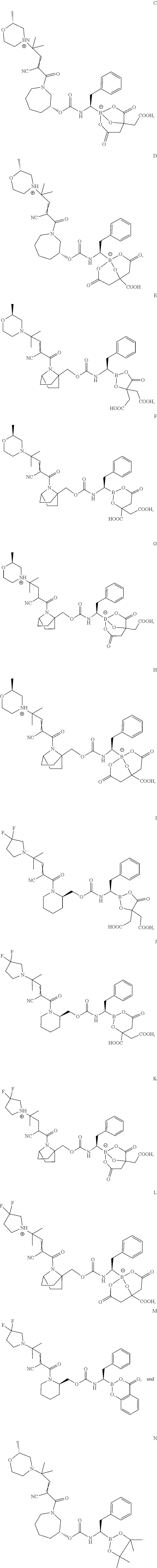

17. A compound according to claim 16 chosen from:

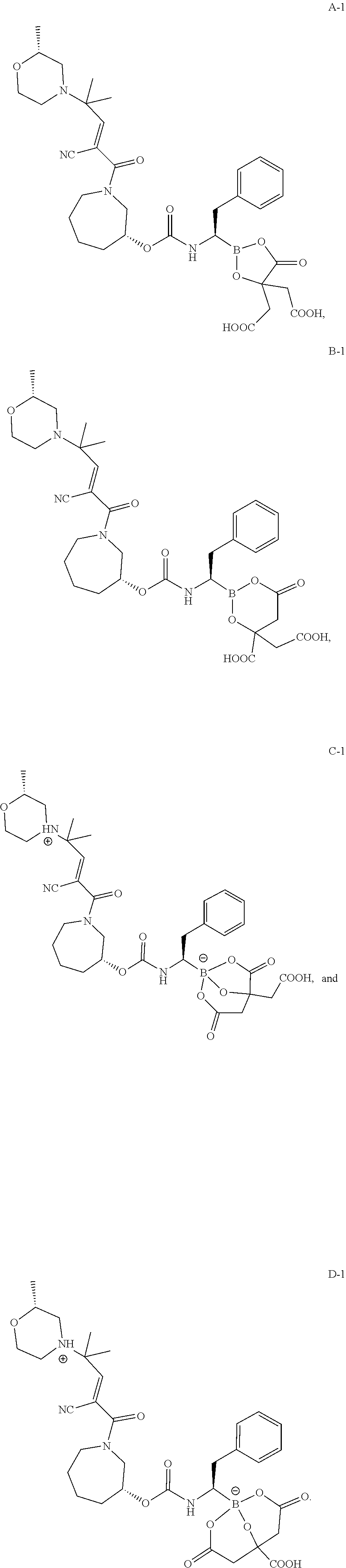 [SMILES: C[C@@H]1CN(C(C)(C)/C=C(\C#N)C(=O)N2CCCC[C@@H](OC(=O)N[C@@H](Cc3ccccc3)B3OC(=O)C(CC(=O)O)(CC(=O)O)O3)C2)CCO1 ; C[C@@H]1CN(C(C)(C)/C=C(\C#N)C(=O)N2CCCC[C@@H](OC(=O)N[C@@H](Cc3ccccc3)B3OC(=O)CC(CC(=O)O)(C(=O)O)O3)C2)CCO1 ; C[C@@H]1C[NH+](C(C)(C)/C=C(\C#N)C(=O)N2CCCC[C@@H](OC(=O)N[C@@H](Cc3ccccc3)[B-]34OC(=O)CC(CC(=O)O)(O3)C(=O)O4)C2)CCO1 ; C[C@@H]1C[NH+](C(C)(C)/C=C(\C#N)C(=O)N2CCCC[C@@H](OC(=O)N[C@@H](Cc3ccccc3)[B-]34OC(=O)CC(C(=O)O)(CC(=O)O3)O4)C2)CCO1]

18. A boronic acid ester of ((R)-1-(((((R)-1-(2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester is chosen from a citric acid ester, a pinacol ester, a malic acid ester, a salicylic acid ester, a lactic acid ester, and a tartaric acid ester.

19. A boronic acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester is chosen from a citric acid ester, a pinacol ester, a malic acid ester, a salicylic acid ester, a lactic acid ester, and a tartaric acid ester.

20. A boronic ester of [(1R)-1-[([[7-(2-cyano-2-[2-methyl-2-[(2S)-2-methylmorpholin-4-yl]propylidene]acetyl)-7-azabicyclo[2.2.1]heptan-1-yl]methoxy]carbonyl)amino]-2-phenylethyl]boronic acid or a pharmaceutically acceptable salt thereof, wherein the ester is chosen from a citric acid ester, a pinacol ester, a malic acid ester, a salicylic acid ester, a lactic acid ester, and a tartaric acid ester.

21. A boronic ester of ((R)-1-(((((R)-1-(2-cyano-4-(3,3-difluoropyrrolidin-1-yl)-4-methylpent-2-enoyl)piperidin-2-yl)methoxy)carbonyl)amino)-2-phenylethyl)boronic acid or a pharmaceutically acceptable salt thereof, wherein the ester is chosen from a citric acid ester, a pinacol ester, a malic acid ester, a salicylic acid ester, a lactic acid ester, and a tartaric acid ester.

22. The boronic ester according to any one of claims 18-21, wherein the ester is a citric acid ester.

23. A boronic acid ester of ((R)-1-(((((R)-1-(2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester converts to its corresponding acid upon exposure to physiological conditions.

24. A boronic acid ester of ((R)-1-(((((R)-1-(2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester is stable at 40° C. and 75% relative humidity for at least three weeks.

25. A boronic ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid or a pharmaceutically acceptable salt thereof, wherein the ester a citric acid ester and is at least about 95% pure.

26. The boronic ester of claim 25, wherein the ester is at least about 99% pure.

27. A crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid, wherein the crystalline form is Crystalline Form I, characterized by an X-ray powder diffractogram comprising at least three peaks chosen from peaks at about 6.6±0.2, 11.2±0.2, 13.2±0.2, 13.8±0.2, 14.3±0.2, 15.6±0.2, 16.8±0.2, 17.5±0.2, 18.5±0.2, and 19.0±0.2 2-θ.

28. The crystalline Form I according to claim 27, characterized by an X-ray powder diffractogram substantially similar to that in FIG. 6.

29. The crystalline Form I according to claim 27 or 28, characterized by a DSC thermogram having a peak endotherm at about 192.7° C.

30. The crystalline Form I according to any one of claims 27-29, characterized by a DSC thermogram showing onset of melting at about 179.5° C.

31. The crystalline Form I according to any one of claims 27-30, characterized by a DSC thermogram substantially similar to that in FIG. 7.

32. The crystalline Form I according to any one of claims 27-31, characterized by a mass loss of less than about 0.5 wt. % between about 25° C. and about 150° C. by thermogravimetric analysis.

33. The crystalline Form I according to any one of claims 27-32, characterized by a TGA thermogram substantially similar to that in FIG. 7.

34. A crystalline form of a citric acid ester of (I-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid, wherein the crystalline form is Crystalline Form II, characterized by an X-ray powder diffractogram comprising at least three peaks chosen from peaks at about 6.1±0.2, 8.1±0.2, 10.6±0.2, 11.0±0.2, 11.9±0.2, 14.0±0.2, 14.7±0.2, 16.6±0.2, 43.7±0.2, and 18.4±0.2 2-θ.

35. The crystalline Form II according to claim 34, characterized by an X-ray powder diffractogram substantially similar to that in FIG. 8.

36. The crystalline Form II according to claim 34 or 35, characterized by a DSC thermogram having a peak endotherm at about 173.7° C.

37. The crystalline Form II according to any one of claims 34-36, characterized by a DSC thermogram showing onset of melting at about 164.9° C.

38. The crystalline Form II according to any one of claims 34-37, characterized by a DSC thermogram substantially similar to that in FIG. 9.

39. The crystalline Form II according to any one of claims 34-38, characterized by a mass loss of about 5.6 wt. % between about 25° C. and about 110° C. by thermogravimetric analysis.

40. The crystalline Form II according to any one of claims 34-39, characterized by a TGA thermogram substantially similar to that in FIG. 9.

41. The crystalline Form I according to any one of claims 27-33 prepared by contacting crystalline Form II according to any one of claims 34-40 with an alcohol or an alcohol mixture.

42. A crystalline form a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid, wherein the crystalline form is Crystalline Form III, characterized by an X-ray powder diffractogram comprising at least three peaks chosen from peaks at about 5.9±0.2, 6.9=0.2,9.4±0.2, 10.5±0.2, 11.1±0.2, 12.3±0.2, 12.7±0.2, 13.8±0.2, 14.3±0.2, and 15.2=0.2 2-θ.

43. The crystalline Form III according to claim 42, characterized by an X-ray powder diffractogram substantially similar to that in FIG. 10.

44. The crystalline Form III according to claim 44 or 43, characterized by a DSC thermogram having a peak endotherm at about 176.5° C.

45. The crystalline Form III according to any one of claim 42-44, characterized by a DSC thermogram having a broad endotherm from about 37.9° C. to about 100° C.

46. The crystalline Form III according to claim 45, wherein said broad endotherm has a peak at about 70.4° C.

47. The crystalline Form III according to any one of claims 42-46, characterized by a DSC thermogram substantially similar to that in FIG. 11.

48. The crystalline Form III according to any one of claims 42-47, characterized by a mass loss of about 3.1 wt. % between about 25° C. and about 105° C. by thermogravimetric analysis.

49. The crystalline Form III according to any one of claims 42-48, characterized by a TGA thermogram substantially similar to that in FIG. 11.

50. The crystalline Form III according to any one of claims 42-49 prepared by a process comprising contacting an alkyl ester, ether and/or toluene with Crystalline Form II according to any one of claims 34-40.

51. The crystalline Form III prepared by the process according to claim 50, wherein the alkyl ester is isopropyl acetate.

52. The crystalline Form III prepared by the process according to claim 50 or 51, wherein the process further comprises isolating Form III from the alkyl ester.

53. A pharmaceutical composition comprising at least one compound according to any one of claims 1-17 and a pharmaceutically acceptable excipient.

54. A pharmaceutical composition comprising at least one boronic ester according to any one of claims 18-26 and a pharmaceutically acceptable excipient.

55. A pharmaceutical composition comprising at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid according to any one of claims 27-52 and a pharmaceutically acceptable excipient.

56. A method of inhibiting Large Multifunctional Protease 2 (LMP2) in a subject comprising administering to said subject in need of said inhibition a therapeutically effective amount of a compound of any one of claims 1-17, at least one boronic ester according to any one of claims 18-26 or at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid according to any one of claims 27-52.

57. A method of inhibiting Large Multifunctional Protease 7 (LMP7) in a subject comprising administering to said subject in need of said inhibition a therapeutically effective amount of a compound of any one of claims 1-17, at least one boronic ester according to any one of claims 18-26 or at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid according to any one of claims 27-52.

58. A method of treating a disease chosen from an autoimmune disorder, an inflammatory disorder, and a hematological disorder in a patient in need of such treatment, comprising administering to the patient a therapeutically effective amount of a compound of any one of claims 1-17, at least one boronic ester according to any one of claims 18-26 or at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid according to any one of claims 27-52.

59. The method of embodiment 58, wherein the disease is chosen from lupus, arthritis including rheumatoid arthritis and psoriatic arthritis, scleroderma, ankylosing spondylitis, Duchene muscular dystrophy (DMD), Becker muscular dystrophy (BMD), idiopathic inflammatory myopathies (IIMs), polymyositis, sporadic inclusion body myositis, dermatomyositis, immune-mediated necrotizing myopathies (IMNM), psoriasis, multiple sclerosis, inflammatory bowel disease, Behçet's disease, ulcerative colitis, Crohn's disease, Sjogren's Syndrome, bronchitis, conjunctivitis, pancreatitis, cholecystitis, bronchiectasis, aortic valve stenosis, restenosis, fibrosis, infection, ischemia, cardiovascular disease, hepatitis, cirrhosis, steatohepatitis, liver inflammation, Alzheimer's Disease (AD), amyotrophic lateral sclerosis (ALS), Huntington's disease, body myositis, myofibrilar myopathy, Graft-versus-Host Disease (GVHD), and multiple myeloma.

60. Use of a composition as a medicament for inhibiting Large Multifunctional Protease 2 (LMP2) in a subject, wherein the composition comprises a therapeutically effective amount of a compound of any one of claims 1-17, at least one boronic ester according to any one of claims 18-26 or at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid according to any one of claims 27-52.

61. Use of a composition as a medicament for inhibiting Large Multifunctional Protease 7 (LMP7) in a subject, wherein the composition comprises a therapeutically effective amount of a compound of any one of claims 1-17, at least one boronic ester according to any one of claims 18-26 or at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid according to any one of claims 27-52.

62. Use of a composition as a medicament for treating a disease in a subject in need therefore, wherein the composition comprises administering to the patient a therapeutically effective amount of a compound of any one of claims 1-17, at least one boronic ester according to any one of claims 18-26 or at least one crystalline form of a citric acid ester of ((R)-1-(((((R)-1-((E)-2-cyano-4-methyl-4-((R)-2-methylmorpholino)pent-2-enoyl)azepan-3-yl)oxy)carbonyl)amino)-2-phenylethyl)boronic acid according to any one of claims 27-52;
and wherein the disease chosen from an autoimmune disorder, an inflammatory disorder, and a hematological disorder.

63. The use according to embodiment 62, wherein the disease is chosen from lupus, arthritis including rheumatoid arthritis and psoriatic arthritis, scleroderma, ankylosing spondylitis, Duchene muscular dystrophy (DMD), Becker muscular dystrophy (BMD), idiopathic inflammatory myopathies (IIMs), polymyositis, sporadic inclusion body myositis, dermatomyositis, immune-mediated necrotizing myopathies (IMNM), psoriasis, multiple sclerosis, inflammatory bowel disease, Behçet's disease, ulcerative colitis, Crohn's disease, Sjogren's Syndrome, bronchitis, conjunctivitis, pancreatitis, cholecystitis, bronchiectasis, aortic valve stenosis, restenosis, fibrosis, infection, ischemia, cardiovascular disease, hepatitis, cirrhosis, steatohepatitis, liver inflammation, Alzheimer's Disease (AD), amyotrophic lateral sclerosis (ALS), Huntington's disease, body myositis, myofibrilar myopathy, Graft-versus-Host Disease (GVHD), and multiple myeloma.
